(19) United States
(12) Patent Application Publication
(10) Pub. No.: US 2026/0174345 A1
(43) Pub. Date: Jun. 25, 2026

(54) WEARABLE OPTICAL PHYSIOLOGICAL MONITORING DEVICE ADHESION

(71) Applicant: iRhythm Technologies, Inc., San Francisco, CA (US)

(72) Inventors: Monica Christine Lin, San Francisco, CA (US); Jeffrey Joseph Abercrombie, II, Oakland, CA (US); John Forbes Black, Kingston, WA (US); James Lee, Los Gatos, CA (US); Sushant Malhotra, Albany, CA (US); Shena Hae Park, San Francisco, CA (US); Thomas Burnell Reeve, III, San Francisco, CA (US)

(21) Appl. No.: 19/127,598

(22) PCT Filed: Nov. 6, 2023
(86) PCT No.: PCT/US2023/078848

§ 371 Date: May 6, 2025

Related U.S. Application Data
(60) Provisional application No. 63/423,756, filed on Nov. 8, 2022.

(51) Int. Cl. A61B 5/024; A61B 5/00

(52) U.S. Cl. CPC A61B 5/02416; A61B 5/02438; A61B 5/6823; A61B 5/6843; A61B 2562/223

(57) ABSTRACT

The present invention relates to a non-invasive cardiac monitoring device that records cardiac data to infer physiological characteristics of a human, such as cardiac arrhythmias or other vital signs. Some examples of the invention allow for long-term monitoring of physiological signals. Further examples allow for processing of ECG and PPG data to calculate pulse arrival time. Some examples include a wearable cardiac monitor device on a chest that includes both the ECG and PPG sensor for long-term adhesion to a mammal for prolonged detection of cardiovascular signals.

### INCORPORATION BY REFERENCE

This application claims priority to U.S. Provisional Application No. 63/423,756, filed on Nov. 8, 2022 and titled “ELECTRICAL COMPONENTS FOR PHYSIOLOGICAL MONITORING DEVICE,” the disclosure of which is hereby incorporated by reference in its entirety and for all purposes herein. Any and all applications for which a foreign or domestic priority claim is identified in the Application Data Sheet as filed with the present application are hereby incorporated by reference under 37 CFR 1.57.

### BACKGROUND

For purposes of this disclosure, certain aspects, advantages, and novel features of various examples are described herein. It is to be understood that not necessarily all such advantages may be achieved in accordance with any particular example. Thus, various examples may be or carried out in a manner that achieves one advantage or group of advantages as taught herein without necessarily achieving other advantages as may be taught or suggested herein.

### FIELD OF THE INVENTION

Disclosed herein are materials, devices, methods, and systems for monitoring physiological signals. For example, such physiological signals may include heart signals, such as an electrocardiogram signal.

### DESCRIPTION OF THE RELATED ART

Abnormal heart rhythms, or arrhythmias, may cause various types of symptoms, such as loss of-consciousness, palpitations, dizziness, or even death. An arrhythmia that causes such symptoms is often an indicator of significant underlying heart disease. It is important to identify when such symptoms are due to an abnormal heart rhythm, since treatment with various procedures, such as pacemaker implantation or percutaneous catheter ablation, can successfully ameliorate these problems and prevent significant symptoms and death. For example, monitors such as Holter monitors and similar devices are currently in use to monitor heart rhythms.

### SUMMARY

The systems, methods and devices of this disclosure each have several innovative aspects, no single one of which is solely responsible for all of the desirable attributes disclosed herein. Details of one or more implementations of the subject matter described in this specification are set forth in the accompanying drawings and the description below.

In some aspects, the techniques described herein relate to an electronic device configured to monitor physiological signals of a user, the electronic device including: a housing at least partially enclosing a circuit board configured to process physiological signals to infer a physiological characteristic of the user; a flexible wing extending from the housing and configured to be affixed to a surface of the user; and an optical sensor assembly positioned on the flexible wing and configured to obtain a photoplethysmography signal, wherein the optical sensor assembly includes an optical emitter configured to emit light and an optical detector configured to receive light, wherein at least a portion of the light emitted by the optical emitter is directed into the skin (for example, the chest) of the user and received by the optical detector from the skin (for example, the chest) of the user, wherein the optical sensor assembly further includes a first directional layer between the optical emitter and the surface of the user and a second directional layer between the optical detector and the surface of the user, and wherein the first directional layer directs light from the optical emitter towards the surface of the user and the second directional layer directs light from the surface of the user towards the optical detector.

In some aspects, the techniques described herein relate to an electronic device, wherein the first directional layer includes a first convex lens and the second directional layer includes a second convex lens.

In some aspects, the techniques described herein relate to an electronic device, wherein the first convex lens and the second convex lens are positioned to extend below the flexible wing such that the first convex lens and the second convex lens press into the skin (for example, the chest) of the user when the electronic device is affixed to the skin (for example, the chest) of the user.

In some aspects, the techniques described herein relate to an electronic device, wherein the optical sensor assembly further includes an opaque barrier positioned between the first directional layer and the second directional layer.

In some aspects, the techniques described herein relate to an electronic device, wherein the opaque barrier blocks at least a portion of light emitted by the optical emitter from being received by the optical detector without passing through the skin (for example, the chest) of the user.

In some aspects, the techniques described herein relate to an electronic device, wherein the opaque barrier is configured to direct at least a portion of light from the surface of the user towards the optical detector.

In some aspects, the techniques described herein relate to an electronic device, further including a reflective layer positioned between the opaque barrier and the skin (for example, the chest) of the user.

In some aspects, the techniques described herein relate to an electronic device, wherein the reflective layer reflects light from the first directional layer.

In some aspects, the techniques described herein relate to an electronic device, wherein the first directional layer and the second directional layer protrudes beyond a portion of the opaque barrier overlapping at least the portion of the opaque barrier and increasing an area of contact with the skin (for example, the chest) of the user.

In some aspects, the techniques described herein relate to an electronic device, wherein the first directional layer and the second directional layer are configured to direct or redirect passage of light of a particular wavelength.

In some aspects, the techniques described herein relate to an electronic device, wherein the first directional layer includes a first concave lens and the second directional layer includes a second concave lens.

In some aspects, the techniques described herein relate to an electronic device, wherein the first concave lens and the second concave lens are formed from a high index material that is index-matched to the stratum corneum of the chest of the user.

In some aspects, the techniques described herein relate to an electronic device, wherein the first directional layer includes a first half ball lens and the second directional layer includes a second half ball lens.

In some aspects, the techniques described herein relate to an electronic device, wherein the first half ball lens and the second half ball lens each include a sapphire half ball lens.

In some aspects, the techniques described herein relate to an electronic device, further including a first adhesive layer configured to affix the first half ball lens to the optical emitter and a second adhesive layer configured to affix the second half ball lens to the optical detector.

In some aspects, the techniques described herein relate to an electronic device, wherein the circuit board includes a flex circuit board.

In some aspects, the techniques described herein relate to an electronic device, further including a backing substrate that exerts pressure on the circuit board to increase contact between the optical emitter and the skin (for example, the chest) of the user, to increase contact between the optical detector and the skin (for example, the chest) of the user, or to increase contact between the optical emitter and the skin (for example, the chest) of the user and the optical detector and the skin (for example, the chest) of the user.

In some aspects, the techniques described herein relate to an electronic device, wherein the optical sensor assembly includes a plurality of optical emitters including the optical emitter, and wherein when the flexible wing is affixed to the surface of the user, the plurality of optical emitters pinch skin (for example, skin on the chest) of the user causing a portion of skin (for example, skin on the chest) to be positioned between the plurality of optical emitters such that the plurality of optical emitters focus light toward the portion of skin (for example, skin on the chest) positioned between the plurality of optical emitters.

In some aspects, the techniques described herein relate to an electronic device, wherein the optical sensor assembly includes a plurality of optical detectors including the optical detector, and wherein when the flexible wing is affixed to the surface of the user, the plurality of optical detectors pinch skin (for example, skin on the chest) of the user causing a portion of skin (for example, skin on the chest) to be positioned between the plurality of optical detectors such that the plurality of optical detectors receive light from the portion of skin (for example, skin on the chest) positioned between the plurality of optical detectors.

In some aspects, the techniques described herein relate to an electronic device, wherein when the flexible wing is affixed to the surface of the user, the optical emitter and the optical detector pinch the skin (for example, skin on the chest) of the user causing a portion of skin (for example, skin on the chest) to be positioned between the optical emitter and the optical detector to direct light from the optical emitter through the skin (for example, skin on the chest) of the user to the optical detector.

In some aspects, the techniques described herein relate to an electronic device, wherein the optical sensor assembly further includes a fiber optical cable or light guide configured to direct light from the optical emitter towards the skin (for example, the chest) of the user.

In some aspects, the techniques described herein relate to an electronic device, further including a spring configured to exert pressure to increase contact between the optical emitter and skin (for example, the chest) of the user and to increase contact between the optical detector and the skin (for example, the chest) of the user.

In some aspects, the techniques described herein relate to an electronic device configured to monitor physiological signals of a user, the electronic device including: a housing at least partially enclosing a circuit board configured to process physiological signals to infer a physiological characteristic of the user; a flexible wing extending from the housing and configured to conform to a surface of the user corresponding to a chest of the user; an optical sensor assembly positioned on the flexible wing and configured to obtain a photoplethysmography signal; and an adhesive layer coupled to a surface of the flexible wing and configured to adhere the electronic device to the surface of the user, wherein the adhesive layer includes an optically clear adhesive layer.

In some aspects, the techniques described herein relate to an electronic device, further including a brightness enhancing film configured to reflect or refract light generated by the optical sensor assembly towards the skin (for example, the chest) of the user.

In some aspects, the techniques described herein relate to an electronic device, wherein the brightness enhancing film is positioned between the adhesive layer and the optical sensor assembly.

In some aspects, the techniques described herein relate to an electronic device, wherein the brightness enhancing film is integrated with the adhesive layer.

In some aspects, the techniques described herein relate to an electronic device, wherein the brightness enhancing film is substantially flush with an optical element of the optical sensor assembly.

In some aspects, the techniques described herein relate to an electronic device, wherein the brightness enhancing film is elevated towards the circuit board providing space to tent skin (for example, skin on the chest) of the user.

In some aspects, the techniques described herein relate to an electronic device, wherein the optical sensor assembly includes a photodiode and a light emitting diode.

In some aspects, the techniques described herein relate to an electronic device, wherein the optically clear adhesive layer couples the photodiode and the light emitting diode to skin (for example, the chest) of the user.

In some aspects, the techniques described herein relate to an electronic device, wherein the optically clear adhesive layer surrounds the photodiode and the light emitting diode without covering the photodiode and the light emitting diode.

In some aspects, the techniques described herein relate to an electronic device, further including a wicking material configured to absorb or evaporate sweat or bodily secretions.

In some aspects, the techniques described herein relate to an electronic device, wherein the wicking material includes a polyester-based material.

In some aspects, the techniques described herein relate to an electronic device, wherein the wicking material is interwoven with the adhesive layer.

In some aspects, the techniques described herein relate to an electronic device, wherein the wicking material is positioned around an optical element of the optical sensor assembly.

In some aspects, the techniques described herein relate to an electronic device, further including channels positioned around an optical element of the optical sensor assembly and configured to permit sweat or bodily secretions to evaporate or escape.

In some aspects, the techniques described herein relate to an electronic device, wherein the channels are within the adhesive layer.

In some aspects, the techniques described herein relate to an electronic device, wherein the circuit board includes a flexible printed circuit board.

In some aspects, the techniques described herein relate to an electronic device, wherein the flexible printed circuit board includes a backing layer with channels that permit sweat or bodily secretions to evaporate or escape.

In some aspects, the techniques described herein relate to an electronic device, wherein the adhesive layer and an optical element of the optical sensor assembly are affixed to a backing layer.

In some aspects, the techniques described herein relate to an electronic device, further including a physical barrier between the adhesive layer and the optical element preventing adhesive leakage from contacting the optical element.

In some aspects, the techniques described herein relate to an electronic device, wherein the physical barrier is tilted towards the adhesive layer.

In some aspects, the techniques described herein relate to an electronic device, wherein the adhesive layer includes a plurality of adhesives.

In some aspects, the techniques described herein relate to an electronic device, wherein the plurality of adhesives includes a first adhesive and a second adhesive, wherein the first adhesive is closer to an optical element of the optical sensor assembly than the second adhesive, and wherein a stickiness of the first adhesive differs from a stickiness of the second adhesive.

In some aspects, the techniques described herein relate to an electronic device, wherein the first adhesive is thinner than the second adhesive.

In some aspects, the techniques described herein relate to an electronic device, further including an adhesive layer at least partially surrounding the optical sensor assembly.

In some aspects, the techniques described herein relate to an electronic device, wherein the adhesive layer at least partially surrounding the optical sensor assembly is not optically clear.

In some aspects, the techniques described herein relate to an electronic device, further including a hydrophobic material or hydrophilic material at least partially surrounding the optical sensor assembly to direct sweat or bodily secretions away from the optical sensor assembly.

In some aspects, the techniques described herein relate to an electronic device, further including perforations positioned around an optical element of the optical sensor assembly and configured to permit sweat or bodily secretions to evaporate or escape.

Examples described herein are directed to a physiological monitoring device that may be worn continuously and comfortably by a human or animal subject for at least one week or more and more typically two to three weeks or more. In some examples, the device is specifically designed to sense and record cardiac rhythm (for example, electrocardiogram, ECG) data, although in various alternative examples one or more additional physiological parameters may be sensed and recorded. Such physiological monitoring devices may include a number of features to facilitate and/or enhance the patient experience and to make diagnosis of cardiac arrhythmias more accurate and timely.

Some examples include an electronic device for monitoring physiological signals in a user, the electronic device comprising: a housing enclosing a circuit board; a wing extending from the housing and configured to conform to a surface of the user, the flexible wing having a bottom surface, a top surface; an electrode coupled to the wing, the electrode in electrical communication with the circuit board and being configured to be positioned in conformal contact with the surface of the user to detect the physiological signals; an optical sensor; and an adhesive layer coupled to the bottom surface of the wing for adhering the electronic device to the user, the adhesive layer having a lower surface, an upper surface interfacing with the bottom surface of the wing, and a thickness between the lower surface and the upper surface. The optical sensor comprises a Light Emitting Diode (LED) and at least one photodiode. The wing has a thickness between the bottom surface and the top surface. The optical sensor comprises at least one emitter and at least one detector. The optical sensor is coupled to the wing. The optical sensor is coupled to another wing. The optical sensor comprises another LED configured to emit light in a different wavelength than the LED. The LED or the other LED is configured to emit a green light. The LED or the other LED is configured to emit an infrared light. The LED or the other LED is configured to emit a red light. The optical sensor comprises at least two photodiodes. A first photodiode illuminates the user's body with infrared light, and a second photodiode illuminates the user's body with a red light. The distance between the LED and the at least one photodiode depends on a wavelength of the light to be emitted from the LED. The distance between the LED and at least one photodiode depends on a desired penetration depth of light into the skin or body of the user. The circuit board is configured to process Electrocardiogram (ECG) data from the electrodes and Photoplethysmogram (PPG) data from the optical sensor to calculate or infer a physiological characteristic of the user. The circuit board is configured to calculate a Pulse Arrival Time (PAT) metric based on the ECG and PPG data. The PAT metric is calculated for each heartbeat. The PAT metric is inversely proportional to the user's blood pressure. The user's blood pressure is calculated based on a relation between peaks and/or troughs in the PPG and ECG data. The electronic device is configured to compare the calculated blood pressure with a baseline blood pressure. The baseline blood pressure is received by the electronic device from a third party blood pressure measurement device. The circuit board is configured to detect peaks and/or troughs in the ECG and PPG data. The circuit board is configured to determine differences in peaks and/or troughs in the ECG and PPG data to calculate the PAT metric. The circuit board is configured to calculate a regression trend of the ECG and PPG data. The regression trend is a linear regression trend. The regression trend correlates ECG and/or PPG and/or impedance data with hemodynamics or other characteristics of the user's blood flow or stroke volume. The electronic device of any preceding claim or other claim herein, wherein the regression trend further tracks changes in behavior of the correlation over ECG or PPG or impedance data values, blood pressure values, and/or time. The electronic device of any preceding claim or other claim herein, wherein the electronic device applies the regression trend to later estimate blood pressure directly from ECG and/or PPG and/or impedance sensor data. The circuit board is configured to calculate a difference in wavelength of light transmitted from the LED and the light received from the photodiode. The circuit board is configured to determine a user's heart quality based on the calculated difference. The electrodes and the optical sensor are disposed on at least one of: the user's chest, the user's extremities, the user's torso, the user's arm, the user's upper arm., the user's torso, the user's chest, the user's shoulder, the user's upper arm, the user's wrist, the user's finger, the user's earlobe, the user's forehead, the user's leg, the user's foot, the user's toe, or the user's blood vessels. The electrodes and the optical sensor collect data from the user's blood vessels. The electrodes and the optical sensor collect data from the same blood vessel. The electrodes and the optical sensor collect data from different blood vessels. The electronic device further comprises an impedance sensor configured to mitigate noise from the collected signals based on detected motion. The electronic device further comprises an impedance sensor configured to measure hemodynamic information. The electronic device further comprises an impedance sensor configured to measure impedance cardiography. The measured impedance cardiography can be applied to improve accuracy of the PAT metric when estimating blood pressure. The electronic device further comprises an impedance sensor configured to measure impedance between a first electrode and a second electrode. The electronic device further comprises an impedance sensor configured to measure impedance between a third electrode and a fourth electrode. The first electrode and second electrode are closer to the center of the chest compared to the third and fourth electrodes. The electrodes are on the chest and aligned with the spine. The electrodes include a first second, third, and fourth electrode. The impedance sensor senses between two or four electrodes. The device applies a current to the first electrode and the second electrode, and the resulting voltages are recorded by the third and fourth electrode. The first and second electrodes are the outer electrodes, and the third and fourth electrodes are the inner electrodes. The current is a low magnitude current. The device comprises a fifth and sixth electrode, wherein the third, fourth, fifth and sixth electrode measure impedance across the user's chest. The first, second, third, and fourth electrodes are aligned with the spine. The fifth and sixth electrodes are not aligned with the spine. The first, second, third, and fourth electrodes are configured to measure impedance. The fifth and sixth electrodes are configured to measure ECG signals. The device or an external computing system is configured to determine an atrial fibrillation burden from the detected signals from the electrodes and/or the optical sensor. The atrial fibrillation burden comprises an amount of time spent in atrial fibrillation by the user during a period of time. The atrial fibrillation burden comprises an amount of time spent in atrial fibrillation by the user during a sleep period and during a wake period. The device or an external computing system is further configured to provide a report, the report comprising the likelihood of the occurrence of cardiac arrhythmia. The report comprises a graph over time of atrial fibrillation burden. The report comprises indications for a presence of atrial fibrillation. The report comprises at least: a 3, 14, or 21 day monitoring period. The electronic device is configured to transmit the detected signals of the electrodes and the optical sensor or a derived signal thereof to an external computing device, the external computing device configured to determine a physiological signal of the user. The external computing system is a server or a gateway. The external computing system is a smartphone. The external computing system communicates with the transmitter through a smartphone intermediary. The electronic device further comprises an accelerometer configured to measure movement of the user. The electronic device is configured to discard recorded physiological data if the data from the accelerometer indicates high movement of the user. The electronic device is configured to remove noise from the recorded physiological data based on the frequency of the movement data. The device or an external computing system is further configured to determine an atrial fibrillation burden, and the atrial fibrillation burden comprises an amount of time spent in atrial fibrillation during movement of the user. The movement of the user comprises a first degree of movement and a second degree of movement. The electrodes and optical sensor are contained within a chest strap. The electrodes and optical sensor are contained within a chest patch. The electrodes and optical sensor are contained within a watch, configured to be worn on a human wrist. The electrodes and optical sensor are contained within a wearable fitness band. The electronic device is configured to detect or infer arrhythmia based on the signals from the electrodes and the optical sensor. The arrhythmia comprises an onset of arrythmia. The arrhythmia comprises a past occurrence of arrythmia. The arrhythmia comprises at least one of: ventricular tachycardia, supraventricular tachycardia, ectopy, ventricular fibrillation, or extended pauses. The housing is configured to be removed from the electronic device and modified while separated from the electronic device. The electronic device is further configured to track an amount of light reflected back to a detector of the PPG sensor. The amount of light reflected back to the detector of the PPG sensor is modulated by pulsing blood flow through the user's vessels. The amount of light reflected back corresponds to hemodynamic information. The electronic device is configured to measure ECG p-waves, ECG R-peaks, measure PPG valleys and/or troughs, measure inflection points along the systolic rise, and/or determine weighted center-of-gravity of each pulse. The electronic device is configured to measure ECG p-waves and infer arrhythmia based on the measured ECG p-waves. The electronic device is configured to measure ECG p-waves and determine PAT based on the measured ECG p-waves. The electronic device is configured to measure a delay between a signal from the electrodes and a signal from the optical sensor. The user's blood pressure is derived based on the measured delay between the signal from the electrodes and the signal from the optical sensor. The electronic device is configured to measure voltage potentials via the electrodes of electrical pathways of the heart, including sinoatrial and/or atrioventricular nodes. The electronic device further comprises a capacitor configured to decouple noise caused by other electrical circuits. The electronic device is configured to measure total electrical conductivity by driving a current between the electrodes, and measuring a voltage between other electrodes. The electrodes are driven by the same input signal. The electrodes comprise a first electrode, a second electrode, a third electrode, and a fourth electrode. The impedance can be sensed between two of the four electrodes. The sensed impedance can be collected on a vertical vector, substantially parallel to the aorta. The sensed impedance can be collected on an angle over the heart. The first electrode and second electrode are disposed above and below the heart, respectively. The third electrode and fourth electrode are disposed to the left side and right side of the heart, respectively. The first electrode and second electrode are disposed to the left side and right side the heart, respectively.

Some examples include an electronic device for monitoring physiological signals in a user, the electronic device comprising: a housing enclosing a circuit board; an electrode coupled to the housing, the electrode in electrical communication with the circuit board and configured to be positioned in conformal contact with the surface of the user to detect the physiological signals; a Light Emitting Diode (LED) and a detector coupled to the circuit board, the LED configured to emit light and the detector configured to detect light; and an adhesive layer for adhering the electronic device to the user.

Some examples include an electronic device for monitoring physiological signals in a user, the electronic device comprising: a housing enclosing a circuit board; an electrode coupled to the housing, the electrode in electrical communication with the circuit board and configured to be positioned in conformal contact with the surface of the user to detect the physiological signals; an emitter and a detector coupled to the circuit board, the emitter configured to emit light and the detector configured to detect light; and an adhesive layer for adhering the electronic device to the user. The electronic device comprises a conformal coating over the LED and the detector. The conformal coating is further applied between the LED and the detector. The conformal coating between the LED and the detector is flush with the coating on the LED or the detector. The conformal coating between the LED and the detector is depressed from the coating on the LED or the detector. The conformal coating is between 10 to 1000 micrometers thin. The conformal coating is between 50 to 500 micrometers thin. The layer of conformal coating on the LED is substantially of similar thickness as the layer of conformal coating on the detector. The electronic device further comprises a barrier between the LED and the detector, wherein the barrier is configured to reduce or eliminate crosstalk between the LED and the detector, wherein the conformal coating is also applied on the barrier. The barrier is an opaque barrier. The opaque barrier prevents or mitigates ambient light external to the electronic device from affecting the LED or the photodiode. The opaque barrier is at least one of: placed adjacent to the LED or the photodiode, in the shape of a dome, placed over the LED or the photodiode, in the shape of a donut and surrounds the LED or photodiode, or is in the shape of a washer and surrounds the LED or photodiode. The electronic device further comprises a reflective layer disposed adjacent to the barrier. The reflective barrier is configured to be in contact with the skin of the patient and prevents another barrier from being in contact with the skin of the patient. The reflective barrier is configured to reflect light exiting the body of the user back into the body of the user. The reflective barrier is configured to reflect light exiting the body of the user away from a detector active area back into the detector active area. The LED is configured to indent the skin of the user. The detector is configured to indent the skin of the user. The electronic device further comprises an additional adhesive layer configured to hold the LED and the detector firmly on the skin of the user. The adhesive layer forms an outer adhesive layer and the additional adhesive layer forms the inner adhesive layer. The outer adhesive layer has a greater thickness than the inner adhesive layer. The outer adhesive layer has a greater stickiness characteristic than the inner adhesive layer. The outer adhesive layer comprises a load bearing adhesive and the inner adhesive layer comprises an optically clear adhesive. The inner adhesive layer comprises a stiffer material than the outer adhesive layer. The distance between the middle of the LED and the edge of the detector closer to the LED is between at least one of: 2-3, 6-8, or 10-30 micrometers. The housing comprises a first mating component and the adhesive layer comprises a second mating component, wherein the housing is configured to be detached or attached from the adhesive layer via the first and second mating components. The detector detects light emitted from the LED that is transmitted through the skin of the user. The detector detects light emitted from the LED that is reflected from the body of the user. The electronic device further comprises a first glass lens configured to direct light from the LED into the skin of the user. The electronic device further comprises a second glass lens configured to direct light from the skin of the user into the detector. The electronic device further comprises a barrier between the first and second glass lenses configured to block at least a portion of the light from passing directly from the LED to the detector. The first or second glass lens comprises ate least one of: a dome shape, a sphere shape, a meniscus shape, or a dome shape curved 180 degrees. The first or second glass lens comprises a dome shape configured to contact the skin surface and another portion that contacts a barrier. The first or second glass lens comprises a dome shape wherein the entire curved portion is configured to contact the skin of the user. The detector is configured to detect at least a portion of the emitted light from the LED that is transmitted from the first glass lens directly through the skin of the user to the second glass lens. The electronic device of any preceding claim or other claim herein, wherein the detector is configured to detect at least a portion of the emitted light from the LED that is transmitted from the first glass lens, modulated by pulsatile blood of the user, and exiting from the user's body to the second glass lens. The electronic device of any preceding claim or other claim herein, wherein the electronic device is further configured to combine the transmitted light and the reflected light and combine the signal to make inferences. The electronic device of any preceding claim or other claim herein, wherein the first glass lens or the second glass lens is configured to indent the skin of the user. The first glass lens and the second glass lens are configured to indent the skin of the user. The amount of indentation is based on the amount of the emitted light transmitted from the first glass lens to the second glass lens. The amount of indentation is based on whether a surface blood vessel is blocked due to the indentation precluding signal capture of the surface blood vessel. The electronic device further comprises a glass lens configured to direct light from the skin of the user into the detector. The electronic device is configured to emit light into the chest of the user. The electronic device is configured to detect light from the chest of the user. The LED and the detector are both placed on the chest, and not the back, of the user. The LED or the detector are configured to indent the skin of the user. The electronic device further comprises an optically clear film or adhesive with wavelength guiding properties configured to redirect light into the skin of the patient at one or more angles closer to the detector. The detector is configured to indent the skin of the user creating a tented area of the skin, wherein the detector is configured to detect light that is passed through the tented portion of the skin by the LED. The detector is placed on a first rail and the LED is placed on a second rail, wherein the detector and LED are elevated from a board via the first and second rail, respectively. The detector is configured to indent one side of the skin of the user and a second detector is configured to indent another side of the skin of the user to create the tented area of the skin, wherein the detector and the second detector are configured to detect light that is passed through the tented area of the skin by the LED. The LED is configured to indent the skin of the user creating a tented area of the skin, wherein the detector is configured to detect light that is emitted by the LED into the tented area of the skin. The LED is configured to indent one side of the skin of the user and second LED is configured to indent another side of the skin of the user creating the tented area of the skin, wherein the detector is configured to detect light that is emitted by the LED and the second LED into the tented area of the skin. The LED is configured to directionally emit light into the tented area of the skin. The wearable device further comprises light piping configured to channel light from the LED and emit light into the tented area of the skin. The light piping is configured to channel light in the direction of the tented area of the skin but not in the opposite direction of the tented skin. The light piping is configured to be index matched between at least two of: the LED, air, skin of the user, or a lens. The LED is a donut shape. The LED is an incomplete donut shape. The electronic device comprises one or more microfluidic channels configured to enable sweat or goop to escape. The one or more microfluidic channels are adjacent to the LED or the detector. The electronic device of any preceding claim or other claim herein, wherein the one electronic device further comprises one or more microfluidic channels configured to enable sweat or goop to evaporate. The electronic device further comprises a contacting portion that applies pressure on a portion of the LED or detector towards the skin of the patient. The electronic device further comprises a wicking material on the contacting portion that is configured to press onto the LED or detector. The electronic device further comprises a physical barrier that blocks overflow of the adhesive layer onto the LED or detector during use of the electronic device over time. At least a portion of the physical barrier is tilted toward the adhesive layer. The LED is configured to emit light in a first wavelength toward the tended area, and the electronic device further comprises second LED configured to emit light in a second wavelength toward the tented area. The detector is configured to detect lights from the LED and the second LED, wherein the electronic device is configured to filter the light from the LED and the light from the second LED. The electronic device further comprises filtering light received at the photodiode. The electronic device is configured to filter light received at the photodiode of a certain wavelength or a range of wavelengths. The electronic device is configured to filter light via a physical filter placed near or adjacent to the photodiode. The electronic device is configured to filter light via signal processing. The detector is configured to detect lights from the LED and the second LED, simultaneously. When the LED is emitting light in the first wavelength, the second LED is turned off. The second LED is configured to indent the skin of the user on the second side of the tented area of the skin, wherein the detector is configured to detect light that is emitted by the LED and the second LED into the tented area of the skin. The second LED is further creating a second tented area of the skin, wherein a second detector is configured to detect light emitted by the second LED into the second tented area. The third LED is further configured to indent the skin of the user helping to creating the second tented area of the skin, wherein the second detector is configured to detect light emitted by the second LED and the third LED into the second tented area. The plurality of LEDs including the LED and a plurality of detectors including the detector are disposed in a circular array. The tented area of the skin is within the inner area of the circular array. The plurality of LEDs including the LED are disposed in a linear array. The plurality of detectors including the detector including the LED are disposed in a linear array. The electronic device further comprises a lens to direct light from the LED to the tented area of the skin. The lens has a first surface not adjacent to the tented area of the skin and a second surface adjacent to the tented area of the skin, wherein the first surface is clearer than the second surface such that more light passes through the first surface than the second surface. The electronic device further comprises a second adhesive layer, wherein the adhesive layer and the second adhesive layer have different stickiness characteristics. The adhesive layer includes one or more openings or slits. The electronic device further comprises one or more wicking materials configured to evaporate sweat or goop. The one or more wicking materials are interwoven with the adhesive. The one or more wicking materials are placed near the adhesive. The electronic device further comprises a flexible layer that rests on top of the LED and the detector, wherein the flexible layer is configured to apply pressure onto the LED and the detector towards the skin of the patient when the electronic device is worn by the user. The electronic device further comprises an opaque barrier disposed between the LED and the detector. The opaque barrier is disposed closer to the LED than the detector. The electronic device further comprises a first set of fiber optics connected to the LED, wherein the first set of fiber optics emit light from the LED into the skin of the user. The electronic device further comprises a second set of fiber optics connected to the photodiode, wherein the second set of fiber optics detect light signals from the skin of the user and pass the signals to the photodiode. The electronic device further comprises brightness enhancing film disposed between the LED and the detector. The electronic device further comprises an optically clear adhesive layer configured to attach the LED to the skin of the user. The electronic device further comprises an optically clear adhesive layer configured to attach the detector to the skin of the user. The electrodes are placed on a different part of the body than the LED or photodiode. The electrodes are held onto the skin of the body via a different part of the adhesive than the LED or photodiode. The electrodes are held onto the skin of the body via a different adhesive than the LED or photodiode. The electronic device further comprises a flexible board, wherein the photodiode and LED are disposed on the flexible board. The flexible board is warped to have protrusions or recessions such that indents or tented areas are formed on the skin when the electronic device is applied to the skin of the patient. The flexible board is flexible to conform to the curvature of the skin. The electronic device further comprises a rigid board, wherein the photodiode and LED are disposed on the rigid board. The electronic device further comprises converting the received light at the photodiode to an electrical signal and transmitting the electrical signal to the housing. The electronic device further comprises transmitting the received light at the photodiode to the housing, and circuitry within the housing configured to convert the light to an electrical signal. The electronic device further comprises a film configured to allow light to pass in one direction and not in the other direction. The film is configured to allow light to pass from the LED but not to the LED. The film is configured to allow light to pass from the photodiode but not to the photodiode. The electronic device further comprises a hydrophilic material below or around the LED or photodiode, wherein the hydrophilic material is configured to pull fluid from the LED or photodiode. The electronic device further comprises a hydrophobic material between the LED and photodiode, wherein the hydrophobic material is configured to move fluid away from both the LED and photodiode. The electronic device further comprises a hydrophobic material over the LED or photodiode. The electrodes are on a first wing and the LED and photodiode are on a second wing. The electrodes are on the same wing as the LED and photodiode. The electronic device is configured to be pressed down by the user while the LED emits light and photodiode collects PPG signals. The electronic device is configured to be pressed down by the user while the electrode collects ECG signals. The electronic device is configured to determine blood pressure subsequent to the user pressing down on the electronic device for a certain time period. The at least a portion of traces for the electrode run parallel with at least a portion of the traces for the optical sensors. The electronic device further comprises a second electrode and a second adhesive, wherein the first adhesive is configured to hold the electrode onto the skin of the user, and the second adhesive is configured to hold the second electrode and one or more optical sensors onto the skin of the user. The first adhesive and the second adhesive are physically separated. The optical components are configured to be detachable from the electronic device. The electronic device is configured to input a signal signature to the LED, and apply a match filter on the light signals received by the photodiode.

Although certain embodiments and examples are disclosed herein, inventive subject matter extends beyond the examples in the specifically disclosed embodiments to other alternative embodiments and/or uses, and to modifications and equivalents thereof.

### BRIEF DESCRIPTION OF THE DRAWINGS

FIGS. 1A-1B illustrate multiple views of an example of a physiological monitoring device. FIG. 1A depicts a perspective view of the physiological monitoring device. FIG. 1B depicts an exploded view of the physiological monitoring device.

FIGS. 2A-2D illustrate multiple views of examples of a physiological monitoring device. FIG. 2A shows a top perspective view, FIG. 2B shows a bottom view, FIG. 2C shows a top perspective view including liners, FIG. 2D shows a bottom view including liners.

FIGS. 3A-3H illustrate various views of examples of a physiological monitoring device. FIG. 3A depicts a perspective view, FIG. 3B shows a top view, FIG. 3C shows a bottom view, and FIG. 3D1 depicts a side view. FIG. 3D2 depicts a side view of a ridge configured for sealing the top and bottom portions of the housing. FIGS. 3E and 3F show a bottom and a top view of the physiological monitoring device with the layers illustrated transparently, to provide visualization through the device. FIGS. 3G and 3H illustrate exploded views of the various components of the physiological monitoring device.

FIG. 4 is a view of an illustrative diagram of a sensor platform is provided, according to one example.

FIGS. 5A, 5B, and 5C provide views of illustrative diagrams according to at least one example.

FIG. 6 is a schematic view of a device according to one example.

FIG. 7 is a schematic view of a device according to one example.

FIG. 8 is a schematic view of an optical sensor according to one example.

FIG. 9 is a view of an illustrative diagram of a user's blood pressure reference according to one example.

FIGS. 10A and 10B provide views of illustrative graphs of a user's ECG data and PPG data according to one example.

FIG. 11 is a view of an illustrative graph of a user's pulse arrival time (PAT) over measurement time according to one example.

FIG. 12A is an example of electrodes to measure electrical conductivity across the thorax according to one example.

FIG. 12B is an example of electrodes to measure electrical conductivity across the thorax and ECG sensors according to one example.

FIG. 13A is a graph of impedance cardiography according to one example.

FIG. 13B is a graph of pulse arrival time determination according to one example.

FIG. 14A illustrates a side view of an example of an optically clear adhesive below the photodiode and the LED according to some examples.

FIG. 14B illustrates a top view of an optically clear adhesive below the photodiode and the LED according to some examples.

FIG. 15A illustrates an example of indenting the skin of a patient according to some examples.

FIG. 15B illustrates an example of indenting the skin of a patient using two extending portions of the wearable device according to some examples.

FIG. 15C illustrates an example of applying fiber optical cable to emit light into an indent of the skin according to some examples.

FIG. 16 illustrates a top view of an example to release sweat or other goop from the electronic components according to some examples.

FIG. 17 illustrates an example with a contacting portion that applies pressure to an LED and/or a photodiode for better coupling with the skin according to some examples.

FIG. 18A illustrates an example of a physical barrier preventing adhesive from overflowing into the electronics according to some examples.

FIG. 18B illustrates an example of a single LED in the shape of a donut or ring according to some examples.

FIG. 19 illustrates an example with LEDs emitting light of different wavelengths according to some examples.

FIG. 20 illustrates an example of repeating LEDs and photodiodes according to some examples.

FIG. 21 illustrates an example of different adhesives for the wearable device according to some examples.

FIG. 22A illustrates an example with a single LED and a single photodiode according to some examples.

FIG. 22B illustrates an example with an array of LEDs and an array of photodiodes according to some examples.

FIG. 22C illustrates an example of LEDs and photodiodes distributed in a circular array according to some examples.

FIG. 23A illustrates an example of a protrusion lined with a lighting channel according to some examples.

FIG. 23B illustrates an example of using a ring or light pipe according to some examples.

FIG. 24 illustrates an example of a leaf spring pressing down on certain components of the wearable device according to some examples.

FIG. 25 illustrates an example of a bowl-shape flexible circuit that depresses into the skin according to some examples.

FIG. 26A illustrates an example of opaque barriers between the LEDs and photodiodes according to some examples.

FIG. 26B illustrates an example of pseudo-transmission directly from the LED to the photodiode through a tented area of skin according to some examples.

FIG. 27A illustrates a cross-sectional view of an example using a bundle of fiber optics for the LEDs and the photodiodes according to some examples.

FIG. 27B illustrates a side view of the example using the bundle of fiber optics according to some examples.

FIG. 28A illustrates an example of skin coupling via two doming compounds according to some examples.

FIG. 28B illustrates another example of skin coupling via two doming compounds according to some examples.

FIG. 28C illustrates an example of circuits 2840, 2860 according to some examples.

FIG. 28D illustrates a skin coupling example using a inward meniscus shape for the glass lens according to some examples.

FIG. 29A illustrates the use of a high lidex material according to some examples.

FIG. 29B illustrates a doming compound that protrudes under the opaque barrier, according to some examples.

FIG. 29C illustrates a ball lens example according to some examples.

FIG. 30A illustrates an example of using waveguides on the surface according to some examples.

FIG. 30B illustrates one example of a waveguide flexible circuit 3006, according to some examples.

FIG. 30C illustrates an example of adhesives used for a dome shape glass lens, according to some examples.

FIG. 30D illustrates an example of adhesive used for an inverse dome shape glass lens, according to some examples.

FIG. 31A illustrates an example of ray tracing of LED emitted light, according to some examples.

FIG. 31B illustrates an example of ray tracing of detected light, according to some examples.

FIGS. 31C, 31D, 31E, 31F, and 31G illustrate examples of applying a circular pedestal on the flex board under the LED/detector, according to some examples.

FIG. 32 illustrates a mold for the sapphire half ball glass lens, according to some examples.

FIG. 33A illustrates a clamp that can clamp down the O-ring, according to some examples. FIG. 33B illustrates a top view of FIG. 33A, according to some examples. FIG. 33C illustrates a side and cross-sectional view of the O-ring, according to some examples.

FIG. 34A illustrates a prototype of a skin coupling example, according to some examples. FIGS. 34B and 34C illustrate examples of different amounts of the optical barrier applied, according to some examples. FIGS. 34D and 34E illustrate the skin coupler in action, according to some examples. FIG. 34F illustrates another example of a skin coupler where multiple fingers can be placed over the LED according to some examples.

FIGS. 35A and 35B illustrate an example of a skin coupling example using conformal coating, according to some examples.

FIG. 36A illustrates an example of how light can be emitted from the emitter onto the skin of a patient, according to some examples. FIGS. 36B and 36D illustrate examples of a photodiode, a detector, and a bridge of epoxy in between according to some examples. FIGS. 36C and 36E illustrate bridges thicker than the bridges of FIG. 36A.

FIGS. 37A and 37C illustrate a first example of a skin coupling example, and FIGS. 37B and 37D illustrate a second example of a skin coupling example.

FIGS. 38A-40B illustrate examples of an adhesive portion mating with an electronic housing, according to some examples.

FIG. 41 illustrates a graph of ECG and PPG signals to determine PPT according to some examples.

FIG. 42 illustrates a graph for calculating PPT as an integral using R-peak information according to some examples.

### DETAILED DESCRIPTION OF EXAMPLES

The following description is directed to a number of various examples. The described examples, however, may be implemented and/or varied in many different ways. For example, the described examples may be implemented in any suitable device, apparatus, or system to monitor any of a number of physiological parameters. For example, the following discussion focuses primarily on long-term, patch-based cardiac rhythm monitoring devices. In one alternative example, a physiological monitoring device may be used, for example, for pulse oximetry and diagnosis of obstructive sleep apnea. The method of using a physiological monitoring device may also vary. In some cases, a device may be worn for one week or less, while in other cases, a device may be worn for at least seven days and/or for more than seven days, for example between fourteen days and twenty-one days or even longer. Many other alternative examples and applications of the described technology are possible. Thus, the following description is provided for exemplary purposes only. Throughout the specification, reference may be made to the term “conformal.” It will be understood by one of skill in the art that the term “conformal” as used herein refers to a relationship between surfaces or structures where a first surface or structure adapts to the contours of a second surface or structure.

Since abnormal heart rhythms or arrhythmias can often be due to other, less serious causes, a key challenge is to determine when any of these symptoms are due to an arrhythmia. Oftentimes, arrhythmias occur infrequently and/or episodically, making rapid and reliable diagnosis difficult. As mentioned above, currently, cardiac rhythm monitoring is primarily accomplished through the use of devices, such as Holter monitors, that use short-duration (less than 1 day) electrodes affixed to the chest. Wires connect the electrodes to a recording device, usually worn on a belt. The electrodes need daily changing and the wires are cumbersome. The devices also have limited memory and recording time. Wearing the device interferes with patient movement and often precludes performing certain activities while being monitored, such as bathing. Further, Holter monitors are capital equipment with limited availability, a situation that often leads to supply constraints and corresponding testing delays. These limitations severely hinder the diagnostic usefulness of the device, the compliance of patients using the device, and the likelihood of capturing all important information. Lack of compliance and the shortcomings of the devices often lead to the need for additional devices, follow-on monitoring, or other tests to make a correct diagnosis.

Current methods to correlate symptoms with the occurrence of arrhythmias, including the use of cardiac rhythm monitoring devices, such as Holter monitors and cardiac event recorders, are often not sufficient to allow an accurate diagnosis to be made. In fact, Holter monitors have been shown to not lead to a diagnosis up to 90% of the time (“Assessment of the Diagnostic Value of 24-Hour Ambulatory Electrocardiographic Monitoring”, by DE Ward et al. Biotelemetry Patient Monitoring, vol. 7, published in 1980).

Additionally, the medical treatment process to actually obtain a cardiac rhythm monitoring device and initiate monitoring is typically very complicated. There are usually numerous steps involved in ordering, tracking, monitoring, retrieving, and analyzing the data from such a monitoring device. In most cases, cardiac monitoring devices used today are ordered by a cardiologist or a cardiac electrophysiologist (EP), rather than the patient's primary care physician (PCP). This is of significance since the PCP is often the first physician to see the patient and determine that the patient's symptoms could be due to an arrhythmia. After the patient sees the PCP, the PCP will make an appointment for the patient to see a cardiologist or an EP. This appointment is usually several weeks from the initial visit with the PCP, which in itself leads to a delay in making a potential diagnosis as well as increases the likelihood that an arrhythmia episode will occur and go undiagnosed. When the patient finally sees the cardiologist or EP, a cardiac rhythm monitoring device will usually be ordered. The monitoring period can last 24 to 48 hours (Holter monitor) or up to a month (cardiac event monitor or mobile telemetry device). Once the monitoring has been completed, the patient typically must return the device to the clinic, which itself can be an inconvenience. After the data has been processed by the monitoring company or by a technician on-site at a hospital or office, a report will finally be sent to the cardiologist or EP for analysis. This complex process results in fewer patients receiving cardiac rhythm monitoring than would ideally receive it.

To address some of these issues with cardiac monitoring, the assignee of the present application developed various examples of a small, long-term, wearable, physiological monitoring device. One example of the device is the Zio® Patch developed and sold by iRhythm Technologies. Various examples of cardiac monitors developed and sold by iRhythm Technologies are also described, for example, in U.S. Pat. Nos. 8,160,682, 8,244,335, 8,150,502, 8,560,046, 8,538,503, 9,241,649, 10,405,799, 10,517,500, 11,141,091, 10,271,754, 10,555,683, 11,051,738, D852,965, D854,167, 9,173,670, 9,451,975, 9,597,004, 9,955,887, 10,098,559, 10,299,691, 10,667,712, 10,813,565, 11,289,197, 11,350,864, 11,350,865, 11,337,632, 11,246,523, 11,399,760, 11,246,524, 11,382,555, 11,083,371, 11,253,185, 11,253,186, 11,375,941, the full disclosures of which are hereby incorporated herein by reference. Generally, the physiological patch-based monitors described in the above references fit comfortably on a patient's chest and are designed to be worn for at least one week and typically two to three weeks. The monitors detect and record cardiac rhythm signal data continuously while the device is worn, and this cardiac rhythm data is then available for processing and analysis.

Such smaller, long-term, patch-based physiological monitoring devices provide many advantages over prior art devices. At the same time, further improvements are desired. One of the most meaningful areas for improvement is to offer more timely notice of critical arrhythmias to managing clinicians. The hallmark of these initial examples was that—for reasons of performance, compliance and cost—the device only recorded information during the extended wear period, with analysis and reporting occurring after the recording completed. Thus, a desirable improvement would be to add the capability of either real-time or timely analysis of the collected rhythm information. While diagnostic monitors with such timely reporting capabilities currently exist, they require one or more electrical components of the system to be either regularly recharged or replaced. These actions are associated with reduced patient compliance and, in turn, reduced diagnostic yield. As such, a key area of improvement is to develop a physiologic monitor that can combine long-term recording with timely reporting without requiring battery recharging or replacement.

Patient compliance and device adhesion performance are two factors that govern the duration of the ECG record and consequently the diagnostic yield. Compliance can be increased by improving the patient's wear experience, which is affected by wear comfort, device appearance, and the extent to which the device impedes the normal activities of daily living. Given that longer ECG records provide greater diagnostic yield and hence value, improvements to device adhesion and patient compliance are desirable.

Signal quality is important throughout the duration of wear, but may be more important where the patient marks the record, indicating an area of symptomatic clinical significance. Marking the record is most easily enabled through a trigger located on the external surface of the device. However, since the trigger may be part of a skin-contacting platform with integrated electrodes, the patient can introduce significant motion artifacts when feeling for the trigger. A desirable device improvement would be a symptom trigger that can be activated with minimal addition of motion artifact.

Further, it is desirable for the device to be simple and cost effective to manufacture, enabling scalability at manufacturing as well as higher quality due to repeatability in process. Simplicity of manufacture can also lead to ease of disassembly, which enables the efficient recovery of the printed circuit board for quality-controlled reuse in another device. Efficient reuse of this expensive component can be important for decreasing the cost of the diagnostic monitor.

There remain clinical scenarios where still longer-duration and lower-cost solutions may be a valuable addition to a portfolio of cardiac ambulatory monitoring options. Inspiration for a potential solution to these needs can be found in the continuous heart rate sensing functionality that is increasingly being incorporated in a variety of consumer health and fitness products, including smart watches and wearable fitness bands. Although continuous heart rate data can be used to provide the user with information about their general fitness levels, it is more both more challenging and valuable to use this data to provide meaningful information related to their health and wellness. For example, the ability to detect potential arrhythmias from continuous heart rate data would enable consumer devices incorporating heart rate sensing functionality to serve as potential screening tools for the early detection of cardiac abnormalities. Such an approach could be clinically valuable in providing a long-term, cost-effective screening method for at-risk populations, for example, heart failure patients at risk for Atrial Fibrillation. Alternatively, this monitoring approach could be helpful in the long-term titration of therapeutic drug dosages to ensure efficaciousness while reducing side effects, for example, in the management of Paroxysmal Atrial Fibrillation. Beyond cardiac arrhythmia detection, the appropriate analysis of heart rate information could also yield insight into sleep and stress applications.

Long-term ambulatory monitoring with a physiologic device, such as an adhesive patch, has a number of clinical applications, particularly when timely information about the occurrence and duration of observed arrhythmias can be provided during the monitoring period. In terms of prevalence, particularly as driven by an aging population, efficiently detecting Atrial Fibrillation (AF) remains the most significant monitoring need. This need is not just evident for patients presenting with symptoms, but also given the increased risk of stroke associated with this arrhythmia for broader, population-based monitoring of asymptomatic AF in individuals at risk due to one or more factors of advanced age, the presence of chronic illnesses like Heart Disease, or even the occurrence of surgical procedures. For the latter group, both perioperative and post-procedure monitoring can be clinically valuable, and not just for procedures targeted at arrhythmia prevention (for example, the MAZE ablation procedure, or hybrid endo and epicardial procedures, both for treatment of AF), but also for general surgeries involving anesthesia. For some applications, the goal of ambulatory monitoring for Atrial Fibrillation will sometimes be focused on the simple binary question of whether AF did occur in a given time period. For example, monitoring a patient following an ablation procedure will typically seek to confirm success, typically defined as the complete lack of AF occurrence. Likewise, monitoring a patient post-stroke will be primarily concerned with evaluating the presence of Atrial Fibrillation.

However, even in those scenarios, if AF occurs, it may be clinically meaningful to evaluate additional aspects to better characterize the occurrence, such as daily burden (% of time in AF each day), and duration of episodes (expressed, for example, as a histogram of episode duration, or as the percentage of episodes that extend beyond a specified limit, say six minutes), both either in absolute terms or in comparison to prior benchmarks (for example, from a baseline, pre-procedure monitoring result). Indeed, measuring daily AF burden, evaluating AF episode duration, and reviewing AF occurrence during sleep and waking periods, and evaluating the presence of AF in response to the degree of a patient's physical movement can be important in a variety of clinical scenarios, including evaluating the effectiveness of drug-based treatment for this arrhythmia.

Making this information available in a timely manner during the monitoring period could allow the managing physician to iteratively titrate treatment, for example, by adjusting the dosage and frequency of a novel oral anticoagulant drug (NOAC) until management was optimized. A further example of this management paradigm is for the patient to be notified of asymptomatic AF—either directly by the device through audible or vibration-based alert, through notification from an application connected to the device, or via phone, email or text-message communication from the managing clinician—for the timely application of a “pill in the pocket” for AF management.

The theme of timely management and/or intervention is certainly evident in situations where clinically significant arrhythmias are observed, for example, asymptomatic second-degree and complete Heart Block, extended pauses, high-rate supraventricular tachycardias, prolonged ventricular tachycaridas, and ventricular fibrillation. For example, the clinical scenario where an extended pause or complete heart block causes Syncope is a particularly significant case where the availability of a timely and dependable monitoring method could reduce or even eliminate the need for in-hospital monitoring of at-risk patients. The theme can also extend to more subtle changes in morphology, for example, QT prolongation in response to medications, which has been shown to have significant cardiac safety implications. Timely awareness of such prolongation could lead, for example, to early termination of clinical studies evaluating drug safety and effectiveness or, alternatively, to adjusting the dosage or frequency as a means to eliminate observed prolongation.

### Physiological Monitoring Devices

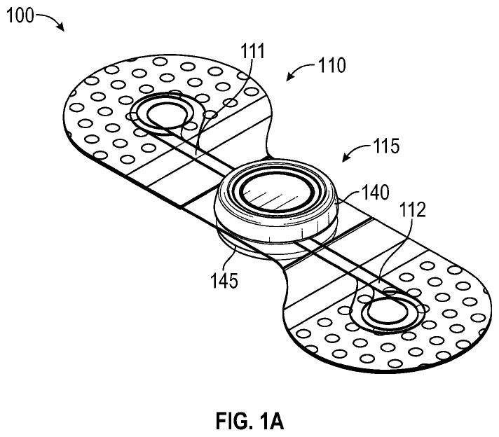

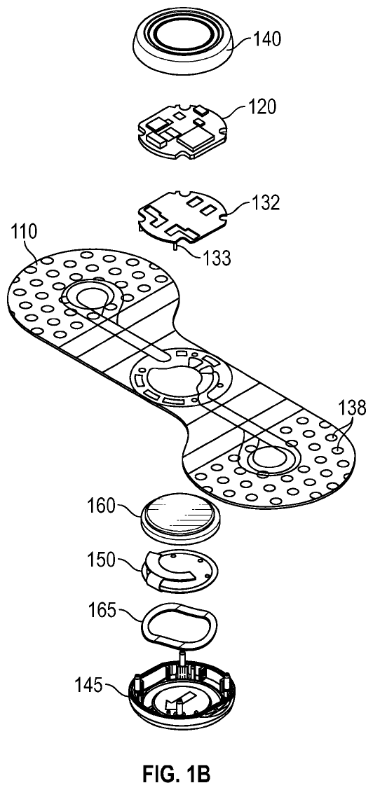

FIGS. 1A-1B illustrate multiple views of a non-limiting example of a physiological monitoring device 100. Additional details of a physiological monitoring device 100 are disclosed in U.S. Pat. No. 11,337,632, which is hereby incorporated by reference in its entirety for all purposes herein. The physiological monitoring device 100 may comprise one or more of the components described elsewhere herein. The physiological monitoring device 100 may comprise a housing 115 comprising an upper housing 140 and a lower housing 145 which are configured to mate together sandwiching a flexible body 110 between the upper housing 140 and the lower housing 145. The flexible body 110 may comprise a trace layer and one or more substrate layers forming the wings of the physiological monitoring device 100. The wings may comprise adhesive layers and electrodes as described elsewhere herein. The rigid body and/or housing 115 may enclose a PCBA 120, a flexible upper housing 140, a battery 160, a battery terminal connector 150, a portion of the trace layer, a spring contact spacer 132, and a spring 165.

FIG. 1A depicts a perspective view of an example of the physiological monitoring device 100. FIG. 1B depicts an exploded view of the physiological monitoring device 100.

The upper housing 140 and the lower housing 145 may sandwich the flexible body 110 as described elsewhere herein. In some examples, the flexible body 110 may comprise one or more apertures 138 through extending through one or more of the substrate layers to provide breathability and moisture management and/or to facilitate drug delivery to the skin of the surface, as described elsewhere herein. An upper gasket layer and/or a lower gasket layer (not shown) may be provided on opposite sides of the flexible body 110. The gasket layers may be adhesive for adhering to the flexible body 110. A compressible seal may be formed above and/or below the flexible body 110. In some implementations, a compressive seal may be formed with the upper housing 140. The upper housing 140 may be a flexible frame. The battery 160 may be positioned below the flexible body 110 comprising the trace layer. The PCBA 120 may be positioned above the flexible body 110 comprising the trace layer. A battery terminal connector 150 may be adhered or otherwise coupled to the battery 160 such that first and second battery traces (not shown) are exposed on an outer surface of the battery terminal connector 150 on a top side of the battery 160. The first and second battery traces may be exposed to the internal volume of the upper housing 140 through a large central opening in the housing area of the trace layer.

Electrical contact between the PCBA 120 and the first and second battery traces and/or electrical contact between the PCBA 120 and electrocardiogram interface portions of the electrical traces 111, 112 may be established by spring contacts. The spring contacts may be coupled to the bottom surface of the PCBA 120. The housing 115 may comprise a spring contact spacer 132 positioned below the PCBA 120. In some examples, the spring contact spacer 132 may be rigidly affixed (e.g., adhered) to the bottom of the PCBA 120. In examples, the spring contact spacer may be attached or integrated into the flexible body 110. In some examples, the spring contact spacer may be integrated into the battery terminal connector. The spring contact spacer 132 may comprise a flat body and a plurality of downward extending legs 133. The legs 133 may be configured to be seated against a top surface and/or a lateral surface of the battery 160, such that the spring contact spacer 132 maintains a minimum separation distance between the battery 160 and the PCBA 120 and provides sufficient space for the spring contacts. The spring contact spacer 132 may comprise one or more holes through which the spring contacts may extend downward from the bottom surface of the PCBA 120. The lower housing 145 may comprise a spring 165, as described elsewhere herein positioned below the battery 160. The spring 165 may bias the battery 160 upward and may bias the first and second battery traces into physical and electrical contact with corresponding spring contacts. The electrocardiogram interface portions of the traces 111, 112 may be seated on a top side of the battery 160 such that biasing the battery 160 upward also biases the electrocardiogram interface portions of the traces 111, 112 into physical and electrical contact with corresponding spring contacts. The substantially consistent spacing between the traces and the PCBA 120 provided by the spring 165 and the spring contact spacer 132 may reduce, minimize, or eliminate noise in the electrical signal caused by fluctuating degrees of electrical contact between the spring contacts and the traces. The assembly may comprise at least one spring contact for each of the first battery trace, second battery trace, first electrical trace 111, and second electrical trace 112. The assembly may comprise more than one spring contacts for some or all of the traces. The spring contacts may be configured under compression induced by the arrangement of the various components, including spring 165, to establish an electrical pathway between each of the traces and the PCBA 120. The compressive contact between the spring contacts and the traces may be maintained even under nominal changes in the separation distances between the traces and the PCBA 120 (e.g., caused by movement) since the spring contacts may extend further downward if the separation distance increases and the biasing corresponding decreases. In some examples, the first and second battery traces may be configured to be positioned on an opposite side of the housing 115 from the first and second electrical traces 111, 112. In some examples, the spring contacts may be configured to carry electrical signals from battery or electrocardiogram signals by contacting electrical traces applied to the upper housing 140 or the lower housing 145. These electrical traces may be applied to the housings through the use of laser direct structuring, plating to a palatable substrate applied in a secondary mold process, or printing via aerosol jet, inkjet or screen printing of conductive materials. In some examples, RF antennas for wireless communication (such as Bluetooth) could be configured through the use of such electrical traces in the upper housing 140 or lower housing 145.

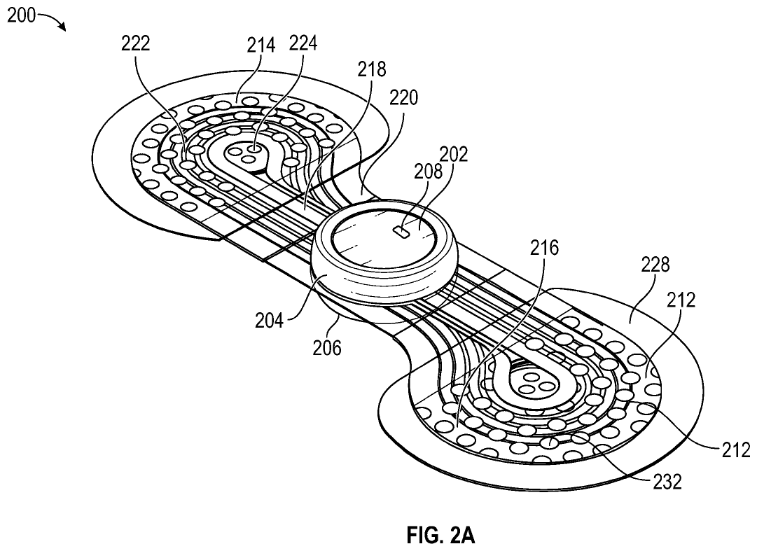

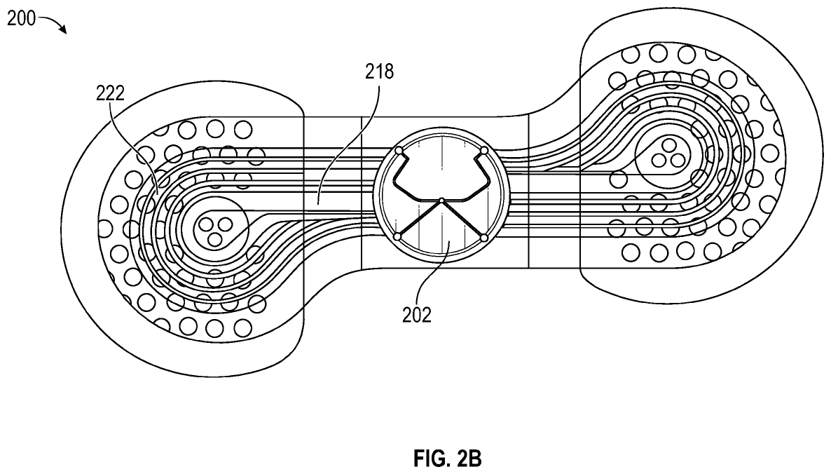

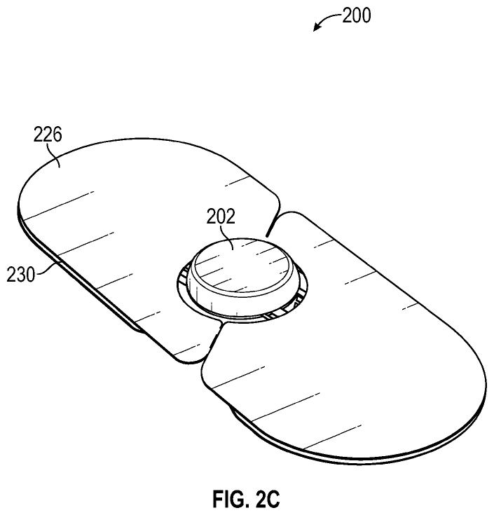

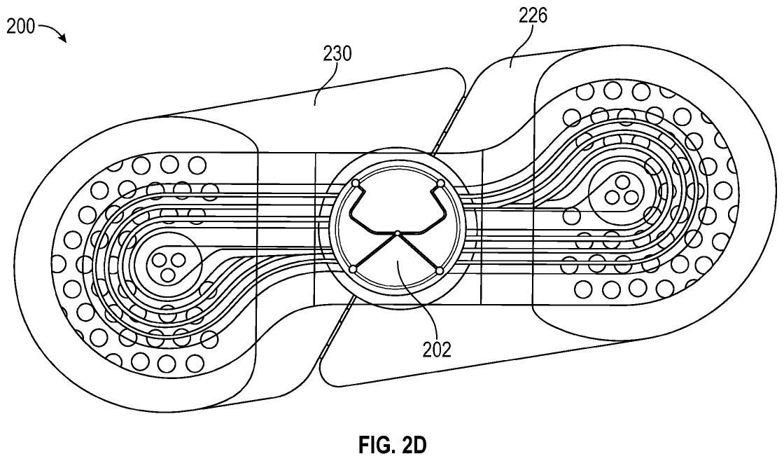

FIGS. 2A-2D depict multiple views of an example of a physiological monitoring device 200, similar to the physiological monitoring devices depicted in FIGS. 1A-1B. Here, the physiological monitoring device includes a central housing 202, comprising an upper housing 204 and a lower housing 206 sandwiched over a flexible substrate. One of skill in the art will understand that the housing may be constructed from any suitable material disclosed herein, such as a rigid polymer or a soft, flexible polymer. In some examples, the housing may include an indicator 208, which may be in any suitable shape such as an oval, a circle, a square, or a rectangle. The indicator may comprise an LED light source (not shown) or any suitable light source, which may be overlain by a transparent or translucent viewing layer positioned against the inner surface of the upper housing. The viewing layer may be constructed from thermoplastic polyurethane or any suitable material. The indicator may be used to indicate a status of the physiological monitoring device such as the battery life of the physiological monitoring device. In some examples, the indicator may indicate whether the physiological monitoring device is collecting data, transmitting data, paused, experiencing an error, or analyzing data. The indicator may display any suitable color, for example red, amber, or green.

Extending outward from the housing are a plurality of wings 212. One of skill in the art will understand that although two wings are depicted here, some examples of the physiological monitoring device 200 may include more than two wings. As explained elsewhere in the specification, the wings may be shaped in such a way to improve adhesion to the skin and retention of the physiological monitoring device against the skin. In examples, the wings may asymmetric, with a greater portion of one wing (an upper lobe) 214 lying above the longitudinal line and a greater portion of another wing lying (a lower lobe) 216 below the longitudinal line, thereby allowing the physiological monitoring device to be positioned diagonally over the heart such that the lower lobe is positioned lower than the heart when a patient is in a standing position.

Extending outward from the housing and contained on or within the wings are electrode traces 218, similar to the electrode traces described elsewhere in the specification, such as with respect to FIG. 1A. As explained elsewhere in the specification, the electrode traces may be printed directly on a flexible substrate which may be part of a multi-layer flexible assembly 220. Additional printed lines 222 may surround the electrode trace 218 for visual enhancement of the physiological monitoring device, however said printed lines 222 may be printed on a different layer than the flexible substrate on which the electrode traces are printed. The printed lines may be printed such that they blend with the shape of the electrode trace. As explained elsewhere in the specification, the electrode trace may encircle a series of breathing holes 224 which allow for air passage to an underlying hydrogel. In examples, there may be one, two, three, four, or more breathing holes. As explained elsewhere in the specification, apertures 232 may extend through one or more layers of the physiological monitoring device to provide breathability and moisture management. In examples, an adhesive border layer 228 may extend outward from the wings, thereby allowing for improved adhesion. FIG. 2B depicts the underside of the physiological monitoring device 200 depicted in FIG. 2A. Here, lower housing 206 is clearly visible as are the electrode traces 218 and printed lines 222 extending outward from the housing. FIGS. 2C and 2D depict the physiological monitoring device 200 of FIGS. 2A-2B, here including an externally facing top liner 226 and skin facing patient release liner 230 overlying the wings and surrounding the central housing 202. Such release liners serve to protect the physiological monitoring device 200 during storage, in particular to protect the adhesive surfaces of the physiological monitoring device. In examples, the liners may be shaped such that two sides meet to form an opening for the housing to extend vertically past the liners.

In some implementations, an abrader may be used to abrade the skin of the patient prior to adhesion of the physiological monitoring device 200 (such as described elsewhere in the specification) to the patient. The abrader may be used to remove a top layer of skin from the patient to improve long-term adhesion of the physiological monitoring device and/or signal quality form the physiological monitoring device.

In various alternative examples, the shape of a particular physiological monitoring device may vary. The shape, footprint, perimeter or boundary of the device may be circular, an oval, triangular, a compound curve or the like, for example. In some examples, the compound curve may include one or more concave curves and one or more convex curves. The convex shapes may be separated by a concave portion. The concave portion may be between the convex portion on the housing and the convex portion on the electrodes. In some examples, the concave portion may correspond at least partially with a hinge, hinge region or area of reduced thickness between the body and a wing.

While described in the context of a heart monitor, the device improvements described herein are not so limited. The improvements described in this application may be applied to any of a wide variety of physiological data monitoring, recording and/or transmitting devices. The improved adhesion design features may also be applied to devices useful in the electronically controlled and/or time released delivery of pharmacological agents or blood testing, such as glucose monitors or other blood testing devices. As such, the description, characteristics and functionality of the components described herein may be modified as needed to include the specific components of a particular application such as electronics, antenna, power supplies or charging connections, data ports or connections for down loading or off-loading information from the device, adding or offloading fluids from the device, monitoring or sensing elements such as electrodes, probes or sensors or any other component or components needed in the device specific function. In addition, or alternatively, devices described herein may be used to detect, record, or transmit signals or information related to signals generated by a body including but not limited to one or more of ECG, EEG and/or EMG. In certain examples, additional data channels can be included to collect additional data, for example, device motion, device flex or bed, heart rate and/or ambient electrical or acoustic noise.

The physiological monitors described above and elsewhere in the specification may further be combined with methods and systems of data processing and transmission that improve the collection of data from the monitor. Further, the methods and systems described below may improve the performance of the monitors by enabling timely transmission of clinical information while maintaining the high patient compliance and ease-of-use of the monitor described above. For example, the methods and systems of data processing and transmission described herein this section of elsewhere in the specification may serve to extend the battery life of the monitor, improve the accuracy of the monitor, and/or provide other improvements and advantages as described herein this section or elsewhere in the specification.

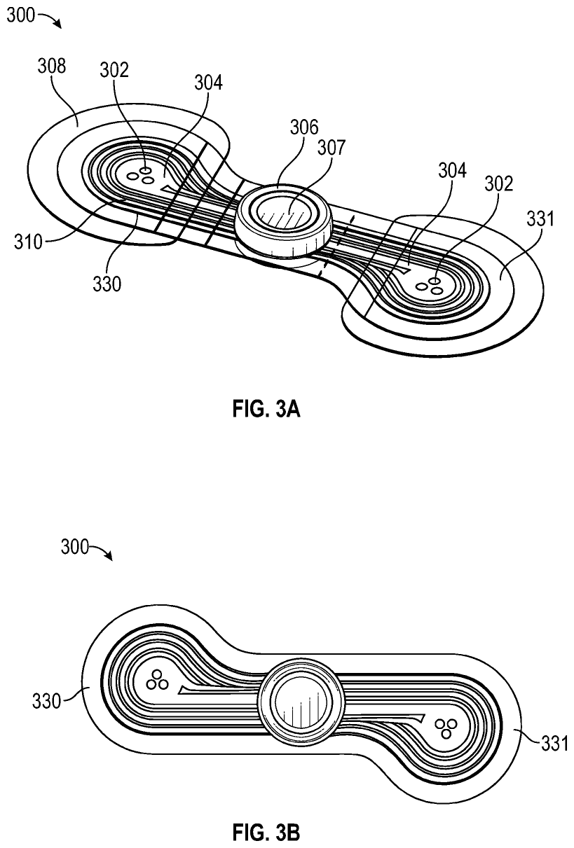

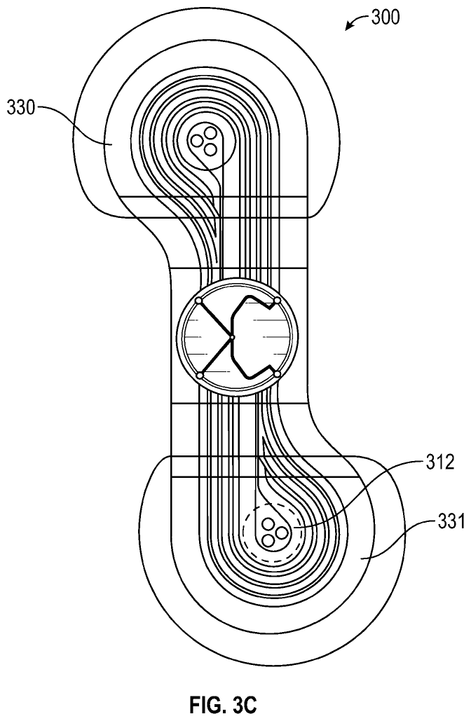

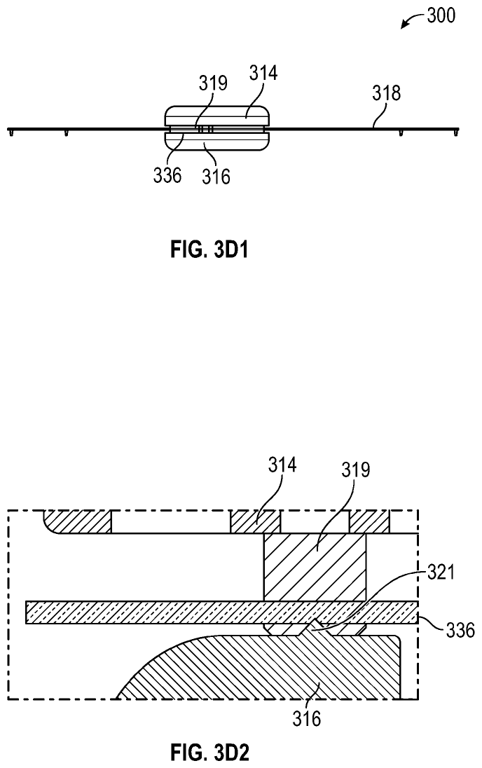

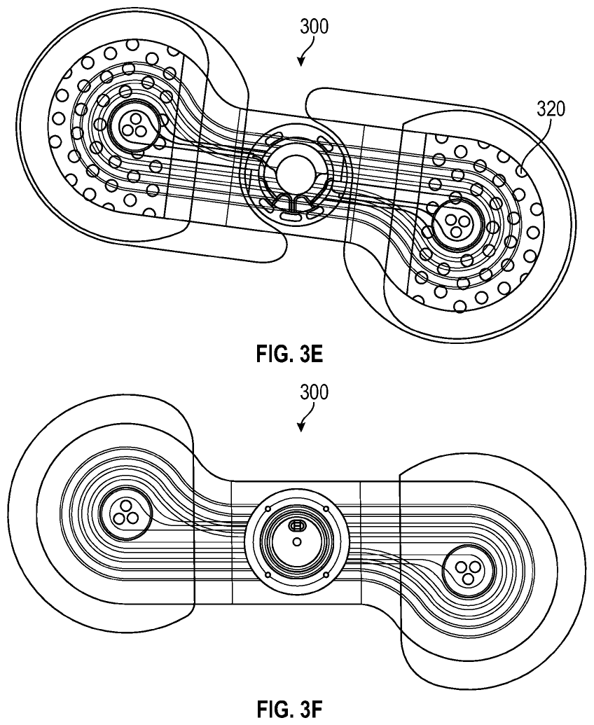

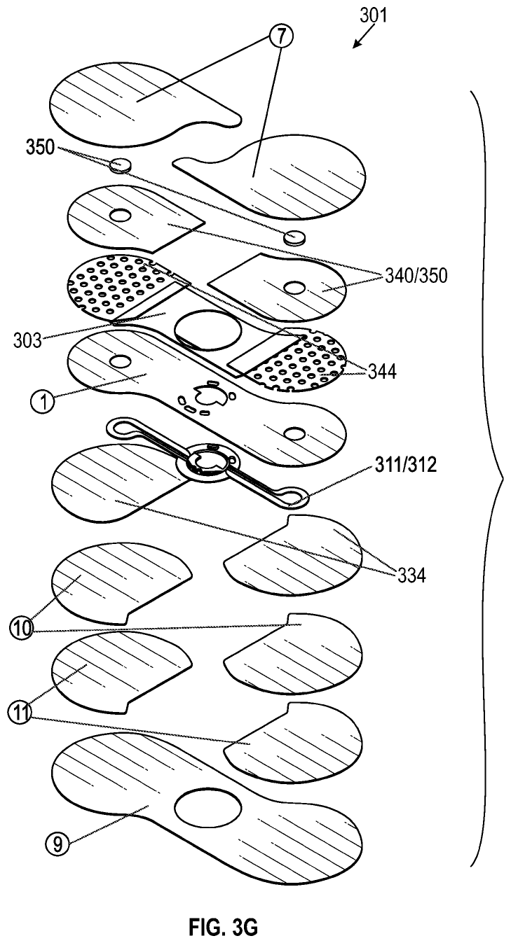

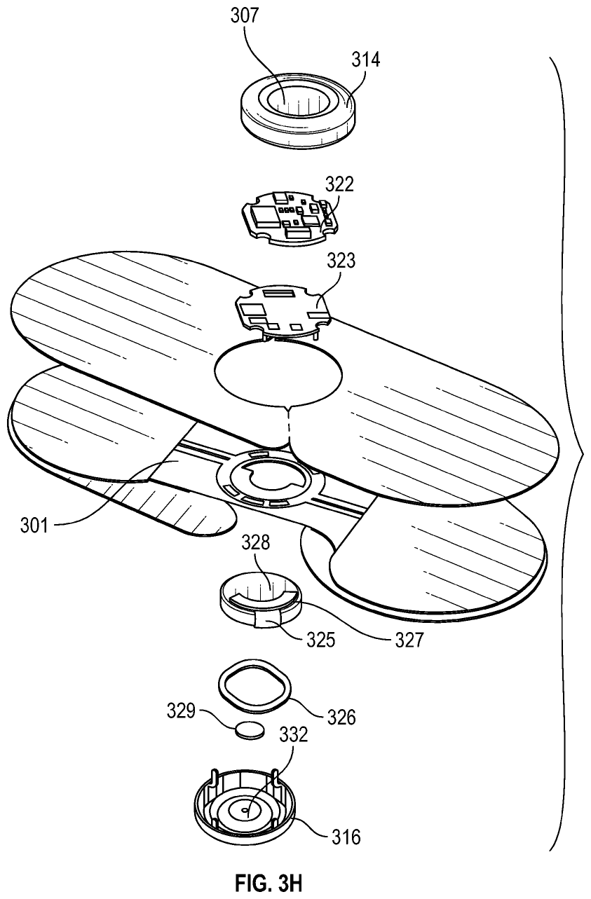

FIGS. 3A-3H depict an example of a physiological monitoring device 300, similar to the physiological monitoring devices depicted in U.S. Pat. No. 11,350,864, which is incorporated by reference in its entirety. FIG. 3A depicts a perspective view of the physiological monitoring device. The physiological monitoring device 300 may comprise wings 330, 331 which are each asymmetrical about a longitudinal axis approximately extending between the electrode interface portions 302 which overlie the electrodes positioned on the underside of the wings. Electrode traces 304, may extend from the housing to the electrodes, to provide electrical communication between the electrode and the central housing. One of the wings 330 may comprise a body which is disproportionately distributed above the longitudinal axis and the other wing 331 may comprise a body which is disproportionately distributed below the longitudinal axis. Therefore, the wings 330, 331, may make the flexible body asymmetric about a transverse axis, perpendicular to the longitudinal axis and extending through the housing 306, which may include patient trigger 307, similar to the other patient triggers disclosed herein this section or elsewhere in the specification. As described elsewhere herein, in certain examples, the patient trigger may encompass about: 10 to 30% of the total top area, such as about 20% of the top area or about 23% such as about 22.8% of the total top area. In certain examples, the patient trigger may encompass more than about 20%, more than about 30%, more than about 40%, more than about 50%, or more than about 75%. In certain examples, the patient trigger may encompass the entire top surface of the housing. The wings 330, 331 may comprise identical shapes which are reversed or flipped about both the longitudinal axis and the transverse axis as shown in FIGS. 3A-3C. In some examples, the wings may be asymmetrical in size and shape, for example the upper wing 330 may be larger than the lower wing 331 or vice-versa. The shapes of the wings 330, 331 may differ such that the relative shape of upper wing 330, differs from the relative shape of lower wing 331. In certain examples, the upper wing 330 may be under greater tension than the lower wing 331 or vice-versa, therefore different sizes and shapes between the two wings may aid in addressing unique force vectors during use of the physiological monitoring device. The configuration of the wings may be particularly suitable for positioning the electrodes in a diagonal arrangement with respect to the height of a subject, therefore potentially reducing peel off due to gravity. One of skill in the art will understand that the orientation of the wings may altered, such that the wings are mirrored, rather than being distributed disproportionately above or below a longitudinal axis. Further, those of skill in the art will understand that the shape of such wings, as described herein, may vary from the generally rounded shapes depicted in FIGS. 3A-3H. For example, the wings may be angular, such as a square shape, rectangular shape, triangular shape, pentagonal shape, or any suitable polygonal shape. These polygonal shapes may have rounded corners to reduce likelihood of peeling from the corner. A liner 308, such as depicted elsewhere herein may be used to cover and protect any adhesive, prior to application of the physiological monitoring device to a patient or user. In examples, the liner may be separated into two parts, one over each wing.

In certain examples, an additional visualization pattern 310 may extend through the wing. The visualization pattern 310 may be in any suitable size or shape to outline the electrode trace and frame the shape of the wings, for example, the visualization pattern 310 may be in the form of lines, such as rounded lines to reflect the contours of the electrode trace and the shape of the wings. In certain examples, there may be one, two, three, four, or more lines. In some examples, the visualization pattern may be formed from a pattern of dots, shapes or other combinations such that the visual cleanliness of the device is maintained as the otherwise clear adhesive layer becomes less visually acceptable to the user through the course of the wear period (e.g., if the adhesive layer picks up foreign material and/or becomes cloudy with absorption of moisture). In certain examples, the visualization pattern may have another functional purpose of alerting the user to how long they have been wearing the device, for example, by changing color over time or wearing down. This change in appearance may alert the user to remove the device at the right time. FIG. 3B shows a top view of an example of the physiological device 300, while FIG. 3C shows a bottom view, and FIG. 3D1 depicts a side view. In FIG. 3C, the flexible electrodes 312 are visible. As shown in FIG. 3D1, upper housing 314 and lower housing 316 portions of the housing may be positioned above and below the flexible body 318. FIGS. 3E and 3F show the underside and topside of the physiological monitoring device 300, with each layer transparent such that all layers are visible. Each layer will be described below in greater detail in the exploded view of the physiological monitoring device 300. Apertures 320 may be positioned in a substrate layer positioned above the adhesive layer. As described above in greater detail, such apertures may provide breathability through one or more layers and may promote transpiration of moisture from below the adhesive layer through the layer or layers comprising the apertures. As shown in FIG. 3D2, in examples, a gasket 319 may be positioned between the upper housing 314 and lower housing 316, co-molded into one or more of the housings. The gasket may compress down on the adhesive assembly and a ridged interface (shown below in FIG. 3D2) or another gasket on the opposite housing to provide waterproofing to the internal electronics hardware. As depicted in FIG. 3B2, a ridge 321 may be positioned on an upper edge of the lower housing 316, the ridge 321 configured to press into the adhesive layer 336. One of skill in the art will understand that the ridge 321 may be of any suitable shape, for example such as an edged ridge as depicted in FIG. 321. In some examples, the ridge may be rounded, square, and/or polygonal. In certain examples, the height of the ridge may be about 0.01 mm to 0.5 mm, about 0.05 mm to 0.4 mm, about 0.1 mm to 0.3 mm, about 0.1 mm to 0.2 mm, or about 0.15 mm such as about 0.13 mm.

FIG. 3G depicts an exploded view of an example of flexible body 301 of the physiological monitoring device 300 described herein this section and elsewhere in the specification. The housing 306 is not shown. As will be understood by one of skill in the art, the image in FIG. 3G is oriented upside down in relation to positioning on the skin. Following the numbering within FIG. 3G, #7 depicts a release liner, which protects the adhesive layer 340 and hydrogel electrodes 350. Directly above the adhesive layer are a perforated layer 344 (containing apertures such as described herein) and a flap layer 303. In certain examples, the perforated layer and flap layers may be constructed from any suitable material, such as polyethylene terephthalate (PET) and/or polyurethane. Directly above the perforated layer may be a lower substrate layer #1, which may be constructed of polyurethane. In examples, the lower substrate layer may have at least one textured side, this side may be positioned such that the textured side faces flap layer #3. In examples, flap layer #3 may also include at least textured side. This textured side may be configured to face lower substrate layer #1. The conductive electrode traces may be printed on an additional, separate substrate (311,312). Or, in some examples, conductive electrode traces may be printed directly on the substrate layer #1. Positioned above the conductive electrode traces may be an upper substrate layer 334. Positioned over the upper substrate layer may be an additional carrier layer #10, followed by an adhesive layer #11 and a topmost rigid liner #9. One of skill in the art will understand that such an arrangement of layers may be applicable to any example of a physiological monitor described herein, such as the examples of FIGS. 3A-3F.

FIG. 3H depicts an exploded view of an example of the housing 306 of the physiological monitor device 300, through which passes flexible body 301, described in detail above. The upper housing cover 314 may include a patient trigger 307. Further, the upper housing cover 314 may encase circuit board 322. Spacer 323, positioned below the circuit board, is configured to maintain consistent spacing between the conductive contact springs that are on the underside of the circuit board and the battery terminals/ECG trace contacts. The spacer may additionally provide electrical insulation between the circuit board and battery. There may be holes in the spacer to allow conductive contact springs to pass through, the contact springs connected to the circuit board. Battery terminal 325, may be positioned below the flexible body 301 and circuit boards 322, thereby overlying wave spring 326. In examples, the battery terminal 325 may be wrapped around and adhered to a coin cell battery 328. The battery terminal 325 may be constructed as a flex circuit with conductive vias 327 that enables the positive underside of the coin cell battery 328 to be brought up to the negative top side of the battery, so that both the negative and positive terminals are presented on the top side of the battery to meet the circuit board contact springs. Alternatively, a battery contact or contacts in the bottom housing can enable the positive underside of the coin cell battery to be brought up to the negative top side to contact the circuit board. Venting layer 329 may be positioned against lower housing 316, over a vent hole 332 in the lower housing. In examples, the venting layer may be constructed from a material that blocks liquid passage while allowing gas passage, for example ePTFE or any other suitable material. The vent hole 332 in combination with the venting layer allows normalization of air pressure between the outside and inside of the housing. In examples, the vent hole 332 in combination with the venting layer prevents button and/or trigger 307 from blowing out or sucking in depending on external air pressure, for example if the patient is at a different altitude such as on a plane. The venting layer may be thin and round with adhesive in a ring configuration on the bottom. The area of the venting layer coated in adhesive may not be gas permeable, while the central portion may be gas permeable but liquid impermeable. The central portion of the venting layer may be positioned over the vent hole, thereby allowing gas passage into and out of the housing while limiting liquid egress and ingress. In certain examples, the venting layer may be integrated into the bottom housing by molding it in, or it could also be ultrasonically welded into the bottom housing, or adhered via any suitable means.

### Physiological Monitoring Device

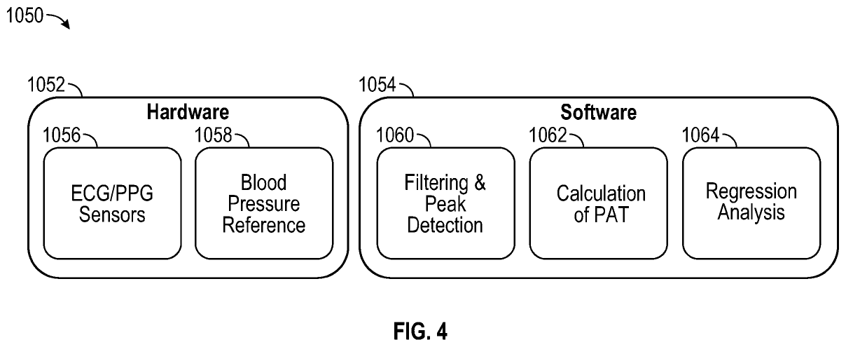

With reference now to the example of FIG. 4, a view of an illustrative diagram of a sensor platform 1050 is provided, according to one example. In some examples, the ECG and/or PPG sensor platform components can include hardware 1052 and software 1054 aspects of the sensor platform. In some examples, the hardware aspects of the sensor platform can include ECG and/or PPG sensors 1056, and a blood pressure reference 1058. In some examples, the blood pressure reference 1058 can measure data of a patient used as a reference for further blood pressure measurements. For example, a patient can measure blood pressure using a blood pressure device and input the blood pressure data into a wireless application, which the sensor platform 1050 can access and compare a currently measured blood pressure for baseline. Additionally, the software components of the sensor platform can be used to perform diagnostic analyses using the data collected from the hardware components. In some examples, the software components of the sensor platform can include filtering and peak detection 1060, calculation of pulse transit time (PTT) or pulse arrival time (PAT) 1062, and regression analysis 1064. In some examples, a separate third party system can be used for such calibration.

In an exemplary example, the filtering and peak detection 1060 can be a first step in pre-processing the data collected from the hardware of the sensor platform. For example, the filtering can include software enabled filtering of data, such as a bandpass filter, Kalman filter, or another comparable filtering process. The peak detection can include software enabled identification of maximum or minimum values from the ECG sensor and/or the PPG sensor. The calculation of the PAT 1062 can include the peaks identified from the peak detection process and using both the ECG sensor data and the PPG sensor data to identify an estimated PAT of each heartbeat. The regression analysis 1064 can be a software enabled trend prediction technique to correlate physiological signals from the sensor platform with hemodynamics and/or other characteristics of the user's blood flow or stroke volume. In some examples, the system can track changes in the behavior of this correlation over sensor values, blood pressure values, or over time. The sensor platform can be configured to use regression techniques such as a linear regression model, closest-fit regression model, time-based regression, multi-variate regression, or any other appropriate regression technique to identify the regression analysis. The example of FIG. 4 is described in a certain order. However it is appreciated that a different order or configuration of software and hardware elements can be applied.

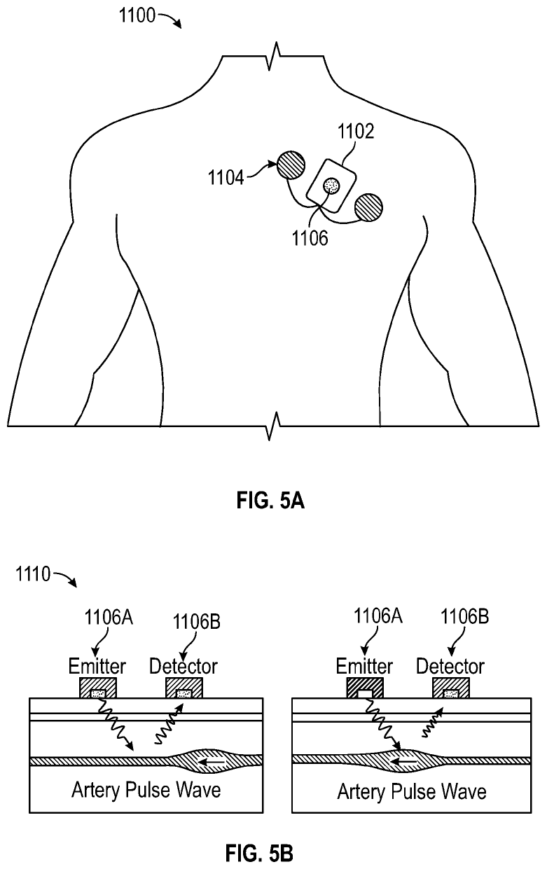

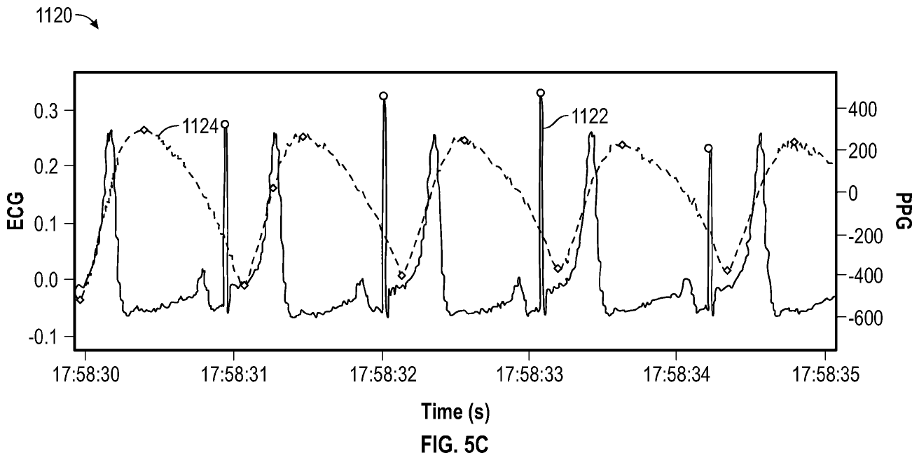

With reference now to the example of FIGS. 5A, 5B, and 5C, providing views of illustrative diagrams of at least one example. In FIG. 5A, the illustrative diagram 1100 can include an overview of a device including the sensor platform 1102 (e.g., sensor platform 1050 disclosed in FIG. 4) to collect data and identify physical or physiological characteristics of a user wearing the device. In one example, the sensor platform 1102 can be worn on the chest of the user as illustrated. In other examples, the sensor platform 1102 can be worn on other areas of the body of the user. In some examples, the sensor platform 1102 can include at least one ECG sensor 1104 and at least one PPG sensor 1106. In some examples, the at least one ECG sensor 1104 and/or the PPG sensor 1106 can be embedded into an adhesive patch making physical contact with the user. In some examples, there are more than one ECG sensor 1104 and/or more than one PPG sensor 1106.

In FIG. 5B, the illustrative diagram 1110 can include an example of the reflective nature of the PPG sensor 1106 on the device. In some examples, the PPG sensor 1106 can include an emitter 1106A and a detector 1106B. For example, the emitter 1106A and the detector 1106B transmit wavelengths of the electromagnetic spectrum into the user's body and receive a reflected wavelength of the electromagnetic spectrum, respectively. In some examples, the system can assess the received signal to track the amount of light reflected back to the detector as modulated by the pulsing blood flow through a user's vessels. The tracked amount of light reflected back can correspond to hemodynamic information. In some examples, the difference in the wavelength spectrum of the transmitted signal and the received signal of the electromagnetic spectrum can correspond to the physical characteristic of the blood travelling in the user's body, which can further correspond to the user's heart quality or condition.

In FIG. 5C, the illustrative diagram 1120 can include an exemplary calculation of the PAT, which can correlate to blood pressure of the user. In some examples, the PAT can be calculated based on data collected from the ECG sensor 1104 and the PPG sensor 1106. For example, the sensor platform 1102 can detect peaks from ECG data of the ECG sensor 1104 and from PPG data of the PPG sensor 1106. The sensor platform 1102 can measure ECG p-waves, measure PPG valleys and/or troughs, inflection points along the systolic rise, determine weighted center-of-gravity of each pulse, and/or the like. The ECG data can correspond with the electrical signals for the heart to pump and the PPG data can correspond to the mechanical pumping of the heart. The measured difference in peaks from ECG signals and PPG signals generally corresponds to a PAT. The values of the PAT can correspond to user's blood pressure. The PAT can be inversely proportional to the user's blood pressure. In some examples, the sensor platform 1102 can measure a time between the peaks identified in the ECG data and the PPG data to calculate a PAT. In some examples, the left axis of the diagram can include ECG values on the ECG measurements 1122 and the right axis can include PPG values on the PPG measurements 1124. The peaks detected for each of the sensors can result in calculating an estimated blood pressure.

With reference now to the example of FIG. 6, this example provides a schematic view of a device 1200 according to one example. This example may be similar to the examples described above in FIGS. 1-5 and several aspects may be the same as in FIGS. 1-5. In some examples, the device 1200 can include at least one photoplethysmography sensor (PPG) sensor 1202, flexible optical electrode traces 1204 coupled to a microprocessor 1206, and at least one ECG sensor 1208. In certain examples, such a device may capture one or more ECG signals and one or more PPG signals to provide comprehensive health information. Similarly to FIGS. 1-5 above, the wings may be flexible and may provide conformal contact with the subject's skin which may prevent the at least one PPG sensor 1202 from peeling or lifting off of the skin, thereby providing strong motion artifact rejection and better signal quality by minimizing transfer of stress to the at least one PPG sensor 1202. Furthermore, in some examples, the device 1200 can include a flexible body (e.g., the flexible body 110 of FIG. 1A) positioned in a configuration with various features that facilitate comfortable wearing of device 1200 by a patient for fourteen (14) days or more without removal.

In some examples, the PPG sensor 1202 can include at least one light-emitting diode (LED) (such as LED 1202A) and a photodiode (PD) (such as PD 1202B). Thus, the LED 1202A and the PD 1202B may collectively be referred to as the PPG sensor 1202. The at least one LED 1202A can illuminate an area of the user where the device 1200 is positioned such that the signal received on the PD provides information about the physiological characteristics of the user's body or blood flow.

In some examples, the system can include a plurality of LEDs. For example, one of the LEDs 1202A can illuminate the user's body with infrared light and another LED (not shown) can illuminate the user's body with red light such that the PD can sense the reflected light from the user's body for both the infrared and red light. In some examples, the system can include one or more LEDs to emit light and one or more photodiodes to detect light.

In certain examples, the reflected light (such as from the infrared, green, red, or other light) can correspond to the user's oxygen saturation, heart rate, respiration, and/or blood pressure. By using dual-wavelength PPG, where the two wavelengths are in different parts of the hemoglobin absorption spectra, a user's oxygen saturation could be derived. In some examples, one wavelength of light can be absorbed to a greater degree by oxy-hemoglobin compared to deoxy-hemoglobin, and another wavelength of light can be absorbed to a greater degree but deoxy-hemoglobin than oxy-hemoglobin. One of the wavelengths could also be absorbed to an equal degree by oxy- and deoxy-hemoglobin. Being able to measure a user's oxygen saturation has applications in monitoring sleep apnea and other respiratory diseases. In some examples, the ECG sensor 1208, the microprocessor 1206, or other processor can convert the reflected light from an analog signal into a digital value. The ECG sensor 1208 can transmit an analog or digital value to the microprocessor 1206 via the flexible optical electrode traces 1204. In some examples, the PPG sensor 1202 can convert the reflected light from an analog signal into a digital value. The PPG sensor 1202 can transmit an analog or digital value to the microprocessor 1206 via the flexible optical electrode traces 1204. In another example, the at least one LED 1202A and/or PD 1202B can follow power constraints for long-term wear by the user. For example, at least one LED 1202A and/or PD 1202B can follow power constraints such that the device 1200 can be worn by the user for 14 days or more without removal.

In some examples, the PPG sensor 1202 can maintain contact with the user at all times while collecting data. For example, the PPG sensor 1202 coupled to an adhesive wing of a patch (e.g., the flexible body 110 in FIG. 1A) can maintain sufficient contact to obtain PPG data. In some examples, the PPG sensor 1202 can measure data from the user's chest. The PPG sensor 1202 coupled to the adhesive wing of the patch can provide sufficient optical coupling for the PPG sensor 1202. In other examples, the device 1200 is agnostic to placement on the user. In some embodiments, the PPG sensor 1202 can be disposed on the underside of the housing that includes the microprocessor 1206. The PPG sensor 1202 in this embodiment may or may not maintain contact with the user's skin at all times. The PPG sensor 1202 and/or the device in this embodiment may or may not include an adhesive coupling component.

In some examples, the microprocessor 1206 can receive the digital value determined at the PPG sensor 1202. In other examples, the microprocessor 1206 can receive analog signal containing information about the reflected light from the user's body via the flexible optical electrode traces 1204. In some examples, the microprocessor 1206 can combine the values from a PPG sensor 1202 with values from an ECG sensor 1208. In some examples, the ECG sensor 1208 and the PPG sensor 1202 can collect data simultaneously and transmit the data values to the microprocessor 1206 at a same time. When the microprocessor 1206 combines the values, the result can be used to calculate a blood pressure of the user. In some examples, the microprocessor 1206 calculates physical parameters from the data. For example, the microprocessor 1206 can calculate the blood pressure based on a relation between peak values of the PPG digital values and the ECG digital values. The relation between the peak values can correspond to a pulse arrival time (PAT). For example, the microprocessor 1206 can detect peaks from ECG data of the ECG sensor 1208 and from PPG data of the PPG sensor 1202. The ECG data can correspond with the electrical signals for the heart to pump and the PPG data can correspond to the mechanical pumping of the heart. The measured difference in peaks from ECG signals and PPG signals generally corresponds to a PAT. The values of the PAT can correspond to user's blood pressure. The PAT can be inversely proportional to the user's blood pressure. In some examples, the microprocessor 1206 can measure a time between the peaks identified in the ECG data and the PPG data to calculate a PAT. In another example, the microprocessor 1206 can transmit the data collected from the ECG sensor 1208 and PPG sensor 1202 to a computer or server for further processing. In another example, the data from ECG sensor 1208 and PPG sensor 1202 (and/or a derived signal thereof) could be downloaded from microprocessor 1206 and uploaded to a computer or server for further processing. For example, the microprocessor 1206 can determine peaks and the computer or server can determine blood pressure of the user.

In some examples, the processing of the data from the ECG sensor 1208 and the PPG sensor 1202 can include filtering and peak detection, calculation of the PAT, and regression analysis. In one example, the microprocessor 1206 can filter the digital signals from the ECG sensor 1208 and the PPG sensor 1202 using a high-pass filter, or another type of filtering method. In another example, the microprocessor 1206 can calculate the PAT using a relation between the data collected from the ECG sensor 1208 and the data collected from the PPG sensor 1202, or between more than one PPG sensor. In some examples, the relation between the PAT metric and the blood pressure can be inversely related. In another example, the microprocessor 1206, computer, or server can calculate a regression trend of the data collected from the ECG sensor 1208 and the PPG sensor with a physiological reference such as blood pressure. The regression trend can be calculated based on linear regression, or any other regression analysis technique. In some examples, the microprocessor 1206, computer, or server can use the regression analysis to then later directly estimate blood pressure value from the ECG and/or the PPG sensor.

Typical PAT measurements from ECG and PPG sensors use physically separated sensors across the patient's body. For example, the ECG sensor on the patient's chest, while the PPG sensor is located on the user's finger. This disclosure recites an example allowing the PAT measurements from the ECG and PPG sensors being located proximate to one another. For example, the ECG and PPG sensors can both be on a device located on the patient's chest. The wearable device and/or other computing device can determine a delay between the ECG and PPG. Such a delay can correlate with a user's blood pressure. For example, the ECG signal can include an electrical signal telling the heart to beat, and the PPG can include a mechanical signal of the physical pumping of blood threw blood vessels. These two signals inherently have a delay (as electrical signals propagate faster than mechanical signals).

In some examples, the ECG sensor 1208 and the PPG sensor 1202 can be proximate to one another, without having one of the sensors being located on the user's extremity. For example, the ECG sensor 1208 and the PPG sensor 1202 can be positioned on the user's chest and provide data collection from the chest location alone. In some examples, the ECG sensor 1208 and the PPG sensor 1202 collect data from blood vessels. The wearable device is configured to measure voltage potentials via the electrodes of electrical pathways of the heart, including at the sinoatrial and/or atrioventricular nodes. Also, the PPG sensor 1202 can collect data on arteries, veins, and/or capillaries.

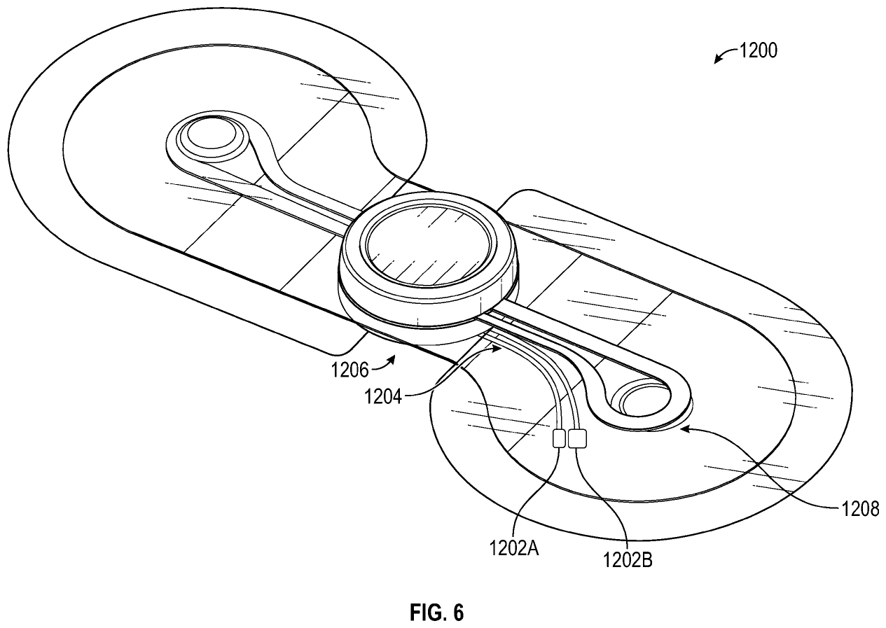

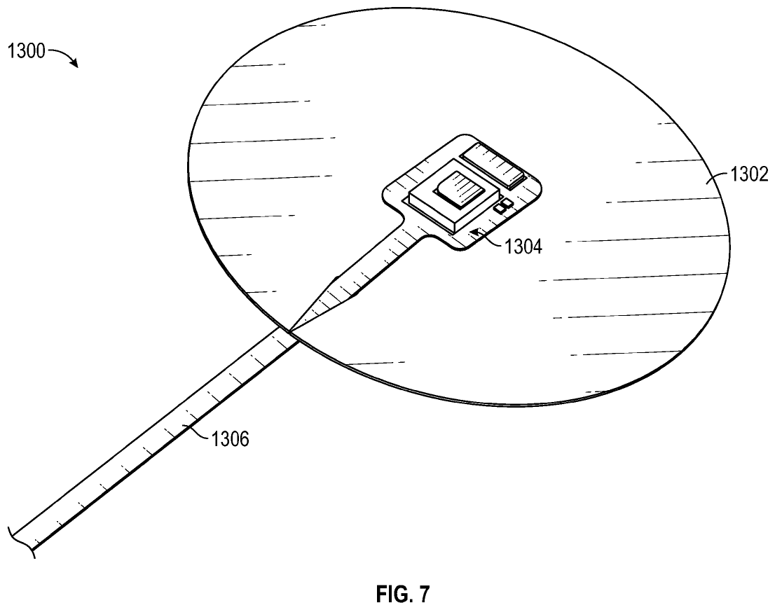

The example of FIG. 7 provides a schematic view of a device 1300 according to one example. In some examples, the device 1300 can include a flexible patch 1302 with a PPG sensor 1304, and flexible optical electrode cable 1306. One advantage is coupling the PPG sensor 1304 with the flexible patch 1302 to result in increased user comfort. Another advantage of integrating optics directly in an adhesive is to improve coupling between the optic sensor and the skin, resulting in improved PPG signal quality. In certain examples, coupling the PPG sensor 1304 with the flexible patch 1302 can result in a user wearing the device 1300 for an extended period of time without discomfort. In some examples, the flexible patch 1302 can include a silicone adhesive material to allow coupling of the device 1300 to a user's body.

In some examples, the wearable device can include an impedance sensor (not shown in the figure). The impedance sensor can include an electric sensor. In some examples, the impedance sensor and the ECG sensor can use different electrodes and/or share at least a subset of the electrodes. The impedance sensor can be used to mitigate noise from a collected analog signal (e.g., if the impedance sensor is used to detect motion), which can result in increased accuracy of data collection measurement. The impedance sensor can also or alternatively be used for impedance cardiography (ICG), such as measuring the electrical conductivity of the torso and/or the torso's changes to derive cardio dynamic parameters including stroke volume and/or time of aortic valve opening. The time of aortic valve opening can be used to help improve the accuracy of the PAT metric when estimating blood pressure.

In some examples, the small size of the PPG sensors 1304 generally may help provide conformal contact with the subject's skin and the flexible electrode cable 1306, by moving the PPG sensor away from the microcontroller and other electronic components (e.g. battery, analog front end), may help prevent the PPG sensor 1304 from peeling or lifting off of the skin, thereby providing strong motion artifact rejection and better signal quality by minimizing transfer of stress to the PPG sensor 1304. Furthermore, the device 1300 can include a configuration and various features that facilitate comfortable wearing of device 1300 by a patient for fourteen (14) days or more without removal. Elements of the device 1300 further allow the flexible patch 1302 to flex freely. The flexible optical electrode cable 1306 can also be thin and flexible, to allow for patient movement without signal distortion.

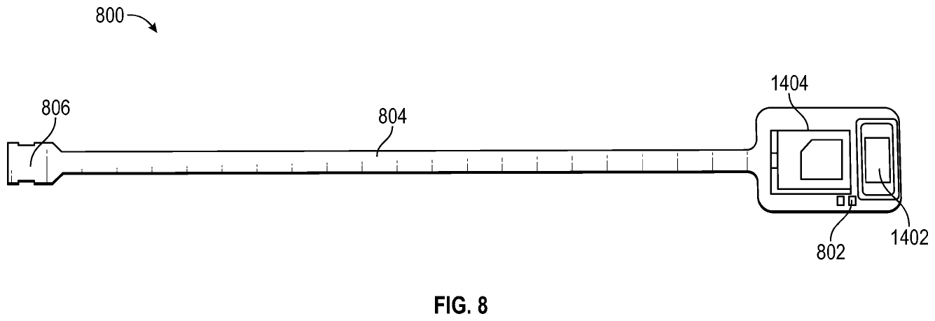

With reference now to the example of FIG. 8, providing a schematic view of an optical sensor 800 according to one example. In some examples, the optical sensor 800 can include at least one LED 1402, at least one photodiode (PD) 1404, a capacitor position 802, a flexible printed circuit (FPC) cable 804, and a cable end 806. A key advantage is a decreased form factor of the device (e.g., the device 1300 in FIG. 7), while increasing device capabilities. In one example, the at least one LED 1402 can include LEDs of a single color or various colors. For example, the at least one LED 1402 can include a green LED with a wavelength of 525 nanometers. In another example, the PD 1404 can include a commercial-off-the-shelf PD able to detect changes in visible light. In some examples, the distance between the at least one LED 1402 and the PD 1404 can be dependent on the wavelength of the at least one LED 1402. For example, the distance between the at least one LED 1402 and the PD 1404 can include less than 10 millimeters. In some examples, the distance between the at least one LED 1402 and the PD 1404 can depend on a desired penetration depth of the light into the skin or body. In one example, the optical sensor 800 can include a capacitor in the capacitor position 802. The capacitor can be used to decouple noise caused by other electrical circuits, which could be picked up along the long FPC cable, from the analog signals of interest. In other examples, the optical sensor 800 has no capacitor in the capacitor position 802. In some examples, the flexible printed circuit cable 804 may be an optical cable that can allow electrical signals to travel from the at least one LED 1402 and PD 1404 to the FPC connector that FPC cable end 806 plugs in to.

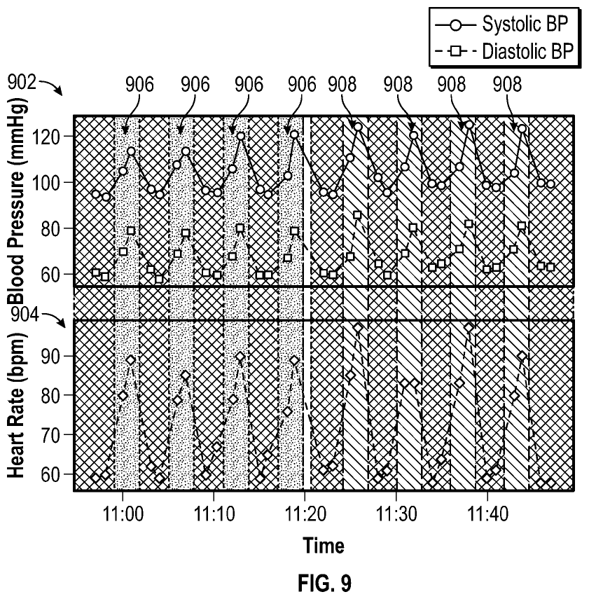

With reference now to the example of FIG. 9, providing a view of an illustrative diagram of a user's blood pressure reference according to one example. The diagram can further include a blood pressure graph 902 and a heart rate graph 904. Each of the graphs can include a first plurality of segments 906 indicating intermediate resistance training. Each of the graphs can include a second plurality of segments 908 indicating difficult resistance training. In some examples, the user's blood pressure measured using various measurement techniques can have a corresponding heart rate of the user during exercise. For example, when the user is exercising with an intermediate resistance band the user's heart rate and blood pressure can correspond with an intermediate baseline of activity. In another example, when the user is exercising with a difficult resistance band the user's heart rate and blood pressure can correspond with a difficult resistance baseline. In each exercise period, the user's heart rate and blood pressure can be greater than a baseline identified when the user is at rest.

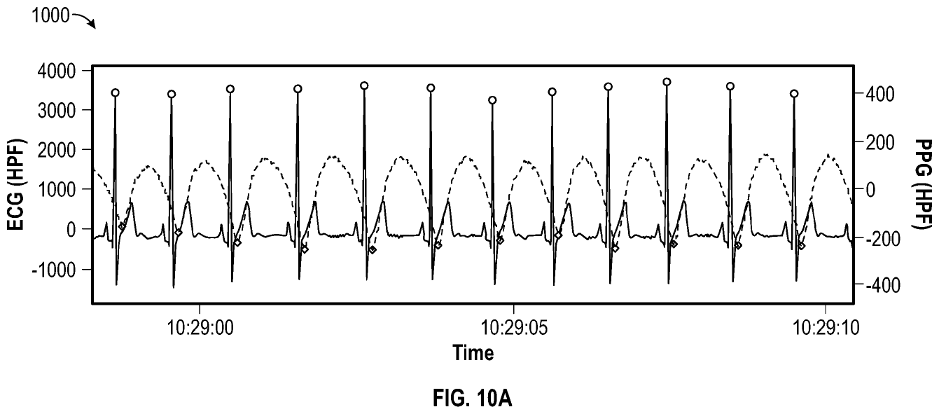

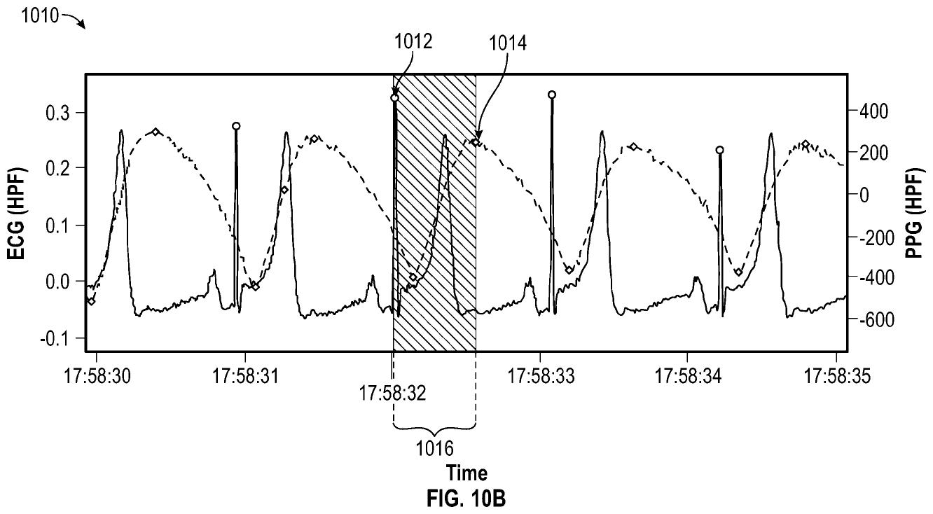

With reference now to the example of FIGS. 10A and 10B, providing views of illustrative graphs of a user's ECG data and PPG data according to one example. In some examples, the user can be wearing a device (e.g., the device 1300 in FIG. 7) including a sensor platform including at least one ECG sensor and at least one PPG sensor. The sensor platform can collect data as the user performs various activities. With reference to the example of FIG. 10A, a first graph 1000illustrates data collected from each of the ECG sensors and the PPG sensors which can correspond to physical qualities of the user's heart such as the electrical signals driving the heart to pump (i.e., the ECG data) and the mechanical pumping of the heart (i.e., the PPG data). With reference to the example of FIG. 10B, a second graph 1010illustrates using both the ECG data and the PPG data, where the device can identify a first peak of the ECG data 1012 and a second peak of the PPG data 1014 on each heartbeat of the data collected. The peaks can then be used by the device (or in post-processing) to identify heart characteristics, such as a pulse arrival time (PAT) 1016 and estimate blood pressure.

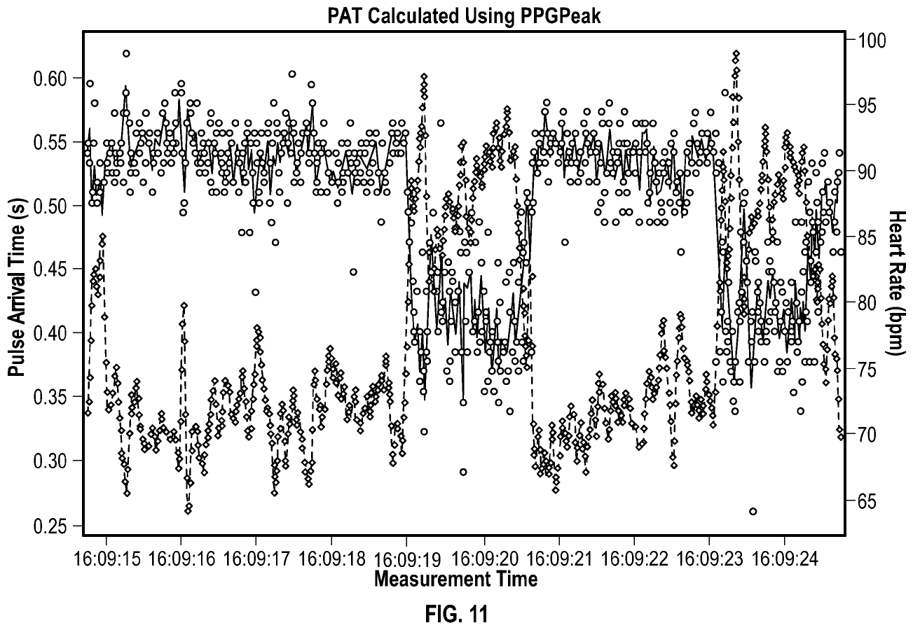

With reference now to the example of FIG. 11, providing a view of an illustrative graph of a user's PAT over time according to one example. In some examples, the PAT can be measured using a device (e.g., the device 1300 in FIG. 7). In some examples, the device (or in post-processing) can cross-reference the PAT with the heart rate of the user to identify accuracy of the measurements. For example, the PAT can be maximum value when the heart rate is at a trough, and conversely, the PAT can be minimum value when the heart rate is at a peak.

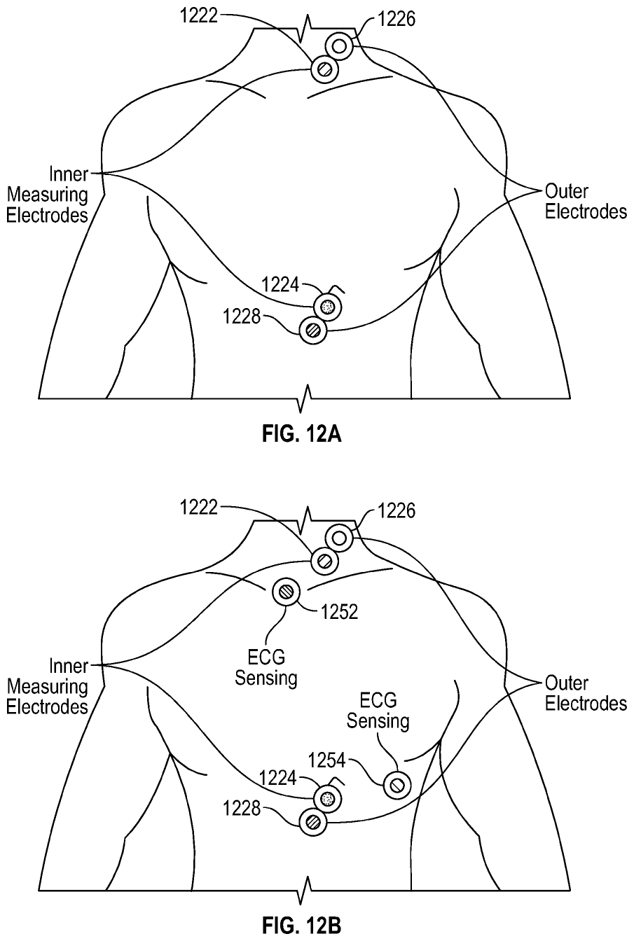

With reference now to the example of FIG. 12A, FIG. 12A is an example of electrodes to measure the impedance, or electrical conductivity, across the thorax according to one example. The device can include a non-invasive sensor that measures electrical signals across the thorax. For example, the device can measure total electrical conductivity using the inner measuring inner electrodes 1222/1224 and/or outer electrodes 1226/1228. The device can measure total electrical conductivity by driving a current between the outer electrodes 1226/1228 and measuring a voltage between the inner electrodes 1222/1224. FIG. 12A illustrates two inner electrodes 1222, 1224 and two outer electrodes 1226/1228. However, it is appreciated that the device can have only inner electrodes, only outer electrodes, a different number of electrodes, a different number of a set of inner/outer electrodes, and/or the like. The device can measure change of electrical signals, such as the total electrical conductivity, over time. The device can apply these measurements to derive cardiodynamic parameters, such as stroke volume, cardiac output, and pre-ejection periods.

In some examples, high frequency, low magnitude current (I) can be applied among electrodes (such as between 2 electrodes) across the chest. The electrodes can be disposed parallel with the spine. The resulting voltage signal (V) can be recorded from the two electrodes. The impedance Z can be calculated from the current I and voltage V. For example, the impedance Z can be calculated by Z=V/I. The current typically seeks the path of least resistance, which could include mainly the blood-filled aorta.

With reference now to the example of FIG. 12B, FIG. 12B is an example of electrodes to measure electrical conductivity across the thorax and ECG sensors according to one example. The device can include electrodes for impedance (such as the two inner electrodes 1222/1224 and two outer electrodes 1226/1228) as well as for ECG (such as electrodes 1252, 1254). The device can use sense electrodes shared with ECG electrodes. The sensors can be set at particular locations, angles, and/or at a minimum or maximum separation of electrodes. The signals emitted from the electrodes can be set at a particular magnitude and/or frequency.

In some examples, impedance can be used to utilize drive and sense electrodes. In a 4-electrode configuration, there could be 2 outer drive electrodes and 2 inner sense electrodes. In a 2-electrode configuration, the signal is driven and sensed from the same set of electrodes. In either configuration, the impedance sense electrodes could be shared with the ECG sense electrodes. Therefore, the electrode configuration could include a plurality of electrodes, such as 2, 4, or 6 electrodes.

In some examples, impedance can be collected on a vertical vector (parallel to the up-and-down nature of the aorta). ECG can be collected on an angle (such as in a 45-degree angle) to measure over the heart. The impedance electrodes can be disposed above and/or below the heart. The ECG electrodes can be disposed to the left and/or right of the heart. For ECG data, the electrodes can be separated over a larger distance while over the heart to produce a better signal quality. For impedance, a larger separation distance can also be applied while being within a maximum distance where the wearable device can no longer be able to resolve the pulsatile waveform from the heart.

In some examples, there are preferred magnitude and frequency values for the input signal (such as the impedance drive signal being above 32 kHz and the current being around 100 uA to 1.5 mA).

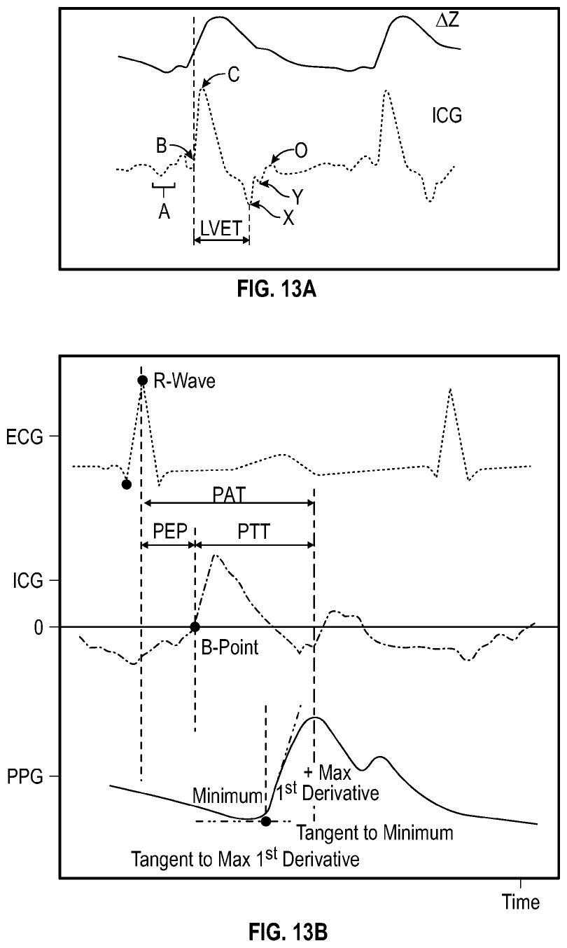

With reference now to the example of FIG. 13A, FIG. 13A is a graph of impedance cardiography according to one example. The device can derive impedance based on a baseline component Z₀ and a time varying component ΔZ. For example, ΔZ is expected to be approximately 0.5% of Z₀. The Impedance Cardiography (ICG) can be a first derivative of ΔZ. In FIG. 13A, A is the contraction of the atrium, B is the opening of the aortic valve, C is the maximum systolic flow, X is the closing of the aortic valve, Y is the closing of the Pulmonal valve, O is the opening of the mitral valve, and LVET is the left ventricular ejection time.

With reference now to the example of FIG. 13B, FIG. 13B is a graph of pulse arrival time determination according to one example. PAT is the pulse transmit time, PEP is the pre-ejection period, PTT is the pulse transit time. PAT includes the time between the electrical initiation of the systole and the onset of left ventricular ejection (PEP). Impedance Cardiography (ICG) is important in that propagation characteristics of arteries can be used to estimate blood pressure. Using ICG can be used to measure blood pressure from the aortic valve opening to arrival of pressure pulse at a distal vessel. PEP can vary with blood pressure, especially in response to short term exercise.

### Examples Related to Skin Coupling

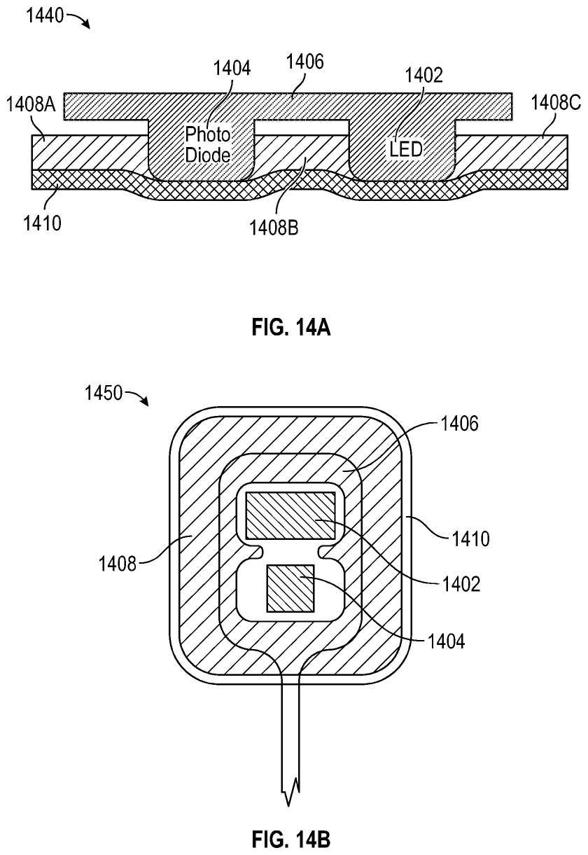

FIG. 14A illustrates a cross sectional view of an example 1440 of an optically clear adhesive below the photodiode and the LED according to some examples. FIG. 14B illustrates a top view 1450 of an optically clear adhesive below the photodiode and the LED according to some examples. The adhesive assembly may be used to adhere a physiological monitoring device to a patient. The example 1440 can include a photodiode 1404, an LED 1402, a flexible circuit 1406 (such as a polyimide flexible circuit), brightness enhancing film 1408A, 1408B, 1408C (collectively referred to herein as brightness enhancing film 1408), and an optically clear adhesive layer 1410. The optically clear adhesive layer 1410 can engage and attach to the user's skin. Because the adhesive layer 1410 is optically clear, the optically clear adhesive layer 1410 can attach directly to the LED 1402 and the photodiode 1404. Advantageously, the LED and the photodiode can couple very well with the skin of the user. In some embodiments, the brightness enhancing film 1408 can be disposed in other locations, such as within the flexible circuit 1406, or overlaid in some or all of the flexible circuit 1406, extend beyond the flexible circuit 1406, and/or the like. The brightness enhancing film 1408 can be integrated with an adhesive that is disposed above the flexible circuit 1406 and applies pressure to the flexible circuit 1406 and the brightness enhancing film 1408 onto the skin of the patient. The brightness enhancing film 1408 and/or the optically clear adhesive layer 1410 can include any type of brightness enhancing film and/or adhesive that is biocompatible and skin friendly. Further, the brightness enhancing film 1408 and/or the optically clear adhesive layer 1410 can include any type of brightness enhancing film and/or adhesive that does not interfere with or that enhances optical signal transmission. Some non-limiting examples of films or adhesives that may be used with certain embodiments described herein include Vikuiti™ Brightness Enhancing Film (BEF) III, Vikuiti™ Enhanced Specular Reflector (ESR), and Contrast Enhancement Film (CEF) 19XX series optically clear adhesives from 3M™.

In some examples, the example 1440 includes a brightness enhancing film 1408 that can refract and/or reflect light to enhance light output. Thus, light that would have otherwise been absorbed by the device and not penetrate the skin could now reflect off of the brightness enhancing film 1408 and penetrate the skin. Moreover, light reflecting from the skin that was not going toward the photodiode could also get another chance by reflecting off of the brightness enhancing film 1408 and returning back to interact with the skin and reflect back into the photodiode. Advantageously, light that may not have otherwise been absorbed by opaque barriers may have another chance of reflecting into the skin or into the photodiode. The brightness enhancing film 1408 can be disposed between the optical components, between an optical component and the skin, underneath opaque layers of the flexible board, and/or locations where light can be reflected. Embodiments described herein include the brightness enhancing film 1408. However, it is appreciated that other reflectors or material that recycle light photons can be used. For example, the brightness enhancing film 1408 can be integrated with, placed adjacent to, and/or replaced with an optically clear adhesive and/or an enhanced specular reflector.

In some examples, the brightness enhancing film 1408 is flush with the photodiode and/or the LED. In other examples, the brightness enhancing film 1408 is elevated toward the circuit 1406 such that the empty space between the photodiode and LED provides space for the skin to tent.

In some examples, adhesives can surround one or more optical components, such as the LED or the photodiode. The adhesive can be applied between the optical components, such as between the LED and the photodiode. The adhesive can include an optically transparent adhesive. The optically transparent adhesive can be applied over one or more of the optical components, such as all of the LEDs and the photodiodes.

In some examples, one or more wicking materials can be integrated with, added onto, placed near, or replace the adhesive. The wicking materials can include a polyester type material that can help absorb and/or evaporate substances, such as goop or sweat. The wicking materials can be placed around the optics or interwoven with the adhesive.

In some examples, a hydrophilic material can be placed around the optics to attract fluid and pull the fluid away from the optics. A hydrophobic material can also be placed (such as between the LED and PD) to move the fluid away from the optics.

In some examples, a light emitting component can be used to emit light and a light detecting component can be used to detect light. For example, a light emitting component can include an LED or an electroluminescent panel. The light detecting component can include a photodiode.

In some examples, the LED and photodiode are in a separate portion of the device than the portion that holds the electrodes. For example, the LED and photodiode can be held down onto the skin of the patient with a different part of adhesive than the adhesive that is holding down the electrodes. FIG. 6 illustrates the optical components 1202A, 1202B and the ECG sensor 1208 being placed on separate parts of the body by the same adhesive but on different parts of the same adhesive.

In some examples, the optical components can be held down by a different part of the same adhesive or by a different adhesive than for the ECG sensor. Advantageously, the optical components can have better coupling on the skin due to the adhesive having to hold a smaller size of components (e.g., only the optical components instead of a larger rigid housing that holds optical components and electrodes).

In some examples, the optical sensors are on the same flexible wing as the electrodes. Advantageously, the device can have a smaller footprint than a device with a separate flexible wing. In some examples, the optical sensors are on a separate flexible wing as the electrodes. Advantageously, having separate flexible wings can improve signal quality by reducing crosstalk between the ECG and PPG signals. Moreover, having a separate flexible wing provides the option to place the PPG sensor in a different and better location on the chest (e.g., more densely populated location of blood vessels) than the optimal location for the ECG sensors.

In some examples, at least a portion of traces for the electrode run parallel with at least a portion of the traces for the optical sensors. Advantageously, the electronic device (e.g., devices that include flexible wings) can have a smaller footprint by reducing the amount of area that the traces use on the flexible wing, resulting in a much smaller footprint on the flexible wing. For example, the portions of the traces that run parallel begin from the housing that holds the electrical circuits and battery.

In some examples, the electronic device comprises a first electrode, a second electrode, a first adhesive and a second adhesive. The first adhesive is configured to hold the first electrode onto the skin of the patient. The second adhesive is configured to hold the second electrode and one or more optical sensors onto the skin of the patient. The first and second adhesives are physically separated to prevent conduction between the two electrodes creating a shorted pathway. The first adhesive can hold a first flexible wing onto the skin of the patient, and the second adhesive can hold a second flexible wing onto the skin of the patient. In some embodiments, the electronic device can include three adhesives. The first adhesive can hold the first electrode onto the skin, the second adhesive can hold the second electrode onto the skin, and the third adhesive can hold one or more optical components onto the skin. In some embodiments, more adhesives can be used, such as a fourth adhesive to hold other optical components onto the skin. In some embodiments, a single adhesive can hold multiple components, such as multiple electrodes.

In some examples, the electronic device is configured to attach and detach the optical sensor onto the device. The optical sensor can be disposable and replaceable. The device can be used and modified to work with only electrodes, only optical sensors, or both. For example, the optical sensor and the trace can be connected to the electronic device by attaching the end of the trace to a connector on the housing. The detachable optical sensor can include its own adhesive separate from the adhesives for the electrodes.

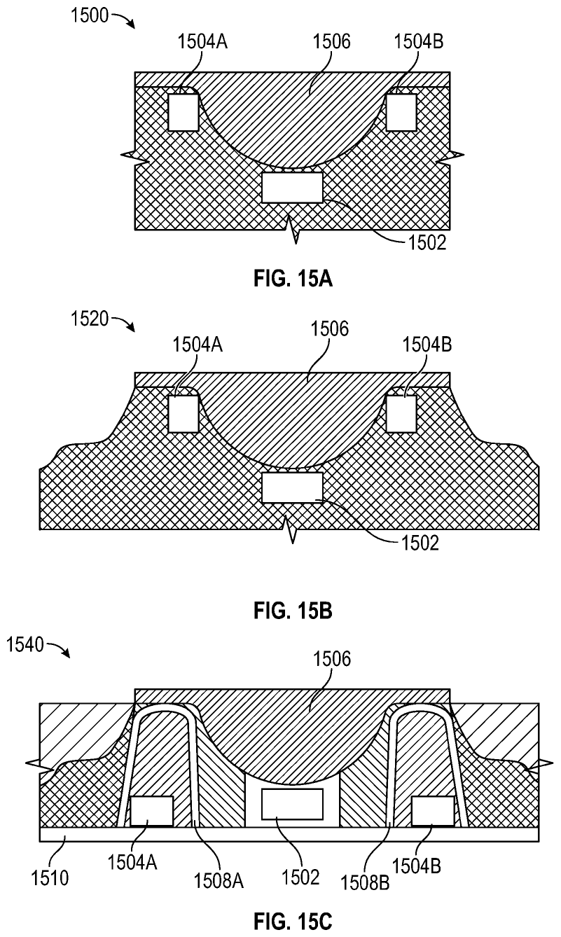

FIG. 15A illustrates an example 1500 of indenting the skin of a patient according to some examples. In the example 1500, the wearable device can include one or more LEDs 1504A, 1504B (collectively referred to herein as LEDs 1504) and a photodiode 1502. The LEDs 1504 can indent the skin 1506 of a user. In the example 1500, two LEDs 1504A, 1504B can indent the skin at different points of the skin. The skin can be tented, such as maintaining a triangular or tentlike appearance when getting pinched from both sides by the LEDs 1504. The LEDs 1504 can emit light into the tented area, and the photodiode 1502 can collect the emitted light from the tented area. The tented area can include a well-vascularized area of tissue. Advantageously, transmitting signals through the tented area can lead to higher signal quality by passing completely through a well-vascularized area of tissue.

Although examples are illustrated as certain components being LEDs, electrodes, or photodiodes, it is appreciated that for FIG. 15A and for other figures herein that these components can be switched, flipped, replaced, and/or swapped with other components (e.g., such as switching the LEDs for photodiodes, and/or vice versa).

The example 1500 of FIG. 15A illustrates a wearable device where the wearable device extends beyond the LEDs 1504 away from the area configured to create the indent on the skin, and this extended area is configured to be flush to the skin of the patient. Thus, the wearable device can include a flush and/or flat surface with a depression where the detector 1502 is.

FIG. 15B illustrates an example 1520 of indenting the skin of a patient using two extending portions of the wearable device. The example 1520 of FIG. 15B illustrates a wearable device where the wearable device does not extend beyond the LEDs 1504 away from the area configured to create the indent on the skin to be flush to the skin of the patient, but instead retracts from the peaks where the LEDs 1504 are located. The wearable device can include protrusions where the LEDs 1504 are located, and depressions where the LEDs 1504 are not located, such as where the photodiode is, or the farther ends of the LEDs from the center tented area.

In some examples, the wearable device can include a plurality of LEDs, such as LED 1504A, 1504B. Advantageously, more light can be emitted into the tented area for the photodetector to detect. More light can interact with the blood and thus more information related to the blood can be received by the photodiode. In other examples, a single LED 1504A can be used. The wearable device can indent the skin on one side via a single LED instead of two sides.

In some examples, the LEDs 1504 can be configured to directionally emit light into the tented area of the skin. In some examples, the LEDs can be directional LEDs that emit light in a particular direction. Advantageously, the LED can focus light toward a certain direction and minimize or eliminate light that would otherwise be emitted away from the tented area.

In other examples, the LEDs 1504 can emit light in multiple directions, and additional material can be used to block or redirect light. For example, an opaque layer can block the light emitting in the opposite direction of the tented area, or a reflective layer can be used to reflect the light emitting in the opposite direction of the tented area to give it another chance to emit in the direction of the tented area of the skin. Moreover, the opaque layer could block ambient light noise from external sources from affecting the LED and/or the photodiode.

In some examples, a uni-directional film or material that allows light to emit in one direction can be applied. For example, the material can be placed on, adjacent to, or near the LED or photodiode. The light emitted from the LED can be emitted toward the material, and the material can allow the light to pass through, whereas light coming from the other side of the material can be blocked. Likewise for the photodiode, light coming from the body can be passed through to the photodiode, whereas light from the other side of the material can be blocked.

FIG. 15C illustrates an example 1540 of applying fiber optical cable to emit light into skin of the user or an indent of the skin according to some examples. In some embodiments, the fiber optical cable or a light guide can focus light towards the skin of the user. The example 1540 can include a backing layer 1510, such as a polyimide backing layer that can include a flexible or regular PCB board. The LEDs 1504 can each be linked to light piping 1508A, 1508B (collectively referred to herein as light piping 1508) to channel light to a side of the protrusion that creates the indent into the skin. The LEDs 1504 can be placed on the backing layer 1510. Advantageously, manufacturing is improved by enabling the LEDs to be placed right on the flat board while enabling light to penetrate into the tented area, where the tented area is elevated from the backing layer.

In some examples, the light piping can be made of or coated with a material with a particular index of refraction, such as an optimal index matching between two of: the LED, air, skin, lenses, and/or the like. For example, a favorable index matching for light transmitted into the skin can be used for light passing from the LED into the skin of the user. The light piping can enable a high percentage of light transfer. In some examples, the light piping can be of a small diameter to enable better flexibility.

In some examples, the light piping 1508 can include one or more opaque layers on portions of the light piping. The opaque layers can prevent light from being emitted in certain parts of the light piping 1508. The portions that would emit light into the tented area of the skin may not include opaque layers. In some examples, the opaque layers can be placed adjacent to, near, or in between optical components.

In some examples, a cup or dome made of or covered in the opaque layer can be placed over one or more of the optical components. In some examples, the opaque layer can be in the form of a washer, a donut, a cylinder with a hollow center, and/or the like surrounding the optical sensor. Advantageously, this opaque layer can prevent ambient light or cross-talk from leading to noise in the electrical signals.

In some examples, the backing layer can include a flexible PCB board. The flexible PCB board can be warped to have protrusions and/or recesses such that indents are formed on the skin and/or tented areas of skin are formed. Advantageously, the LEDs and/or the photodiodes can be disposed closer to the tented area while still being manufactured to be placed on the backing layer.

In some examples, the flexible board can be bent during use to conform to the curvature of the user's skin. The flexible board can be thinner than other boards. Such flexibility can enable better coupling with the skin of the patient.

In some examples, the LED and/or the photodiode are disposed on a rigid board. Advantageously, the components are more stable, such that the distances between the optical components are less susceptible to variability. Moreover, a rigid board could improve indentation into the skin.

FIGS. 15A, 15B, and 15C show a cross-sectional view of the electronic device. In some examples, the LEDs and/or the photodiode can be rectangular shaped. In other examples, the LEDs and/or the photodiode can be circular. For example, the photodiode 1502 can be a sphere, a cylinder, an oval sphere, and/or the like.

In some examples, the LEDs 1504A, 1504B can be a part of a single LED that is a donut or ring shape. The LED shape can comprise a single LED that is in the shape of a donut or ring and/or can comprise multiple LEDs that form a donut or ring shape. In some examples, the LED or the photodiode can be in the shape of an incomplete donut or ring where only a certain percentage or portion of the LED is in a donut or ring shape. For example, the LED could be in a ring shape for 120, 180, 220, 260, or 300 degrees and the remaining area could not include an LED. Advantageously, the incomplete portion could relieve some of the pinching that could occur from the LED indenting into the skin.

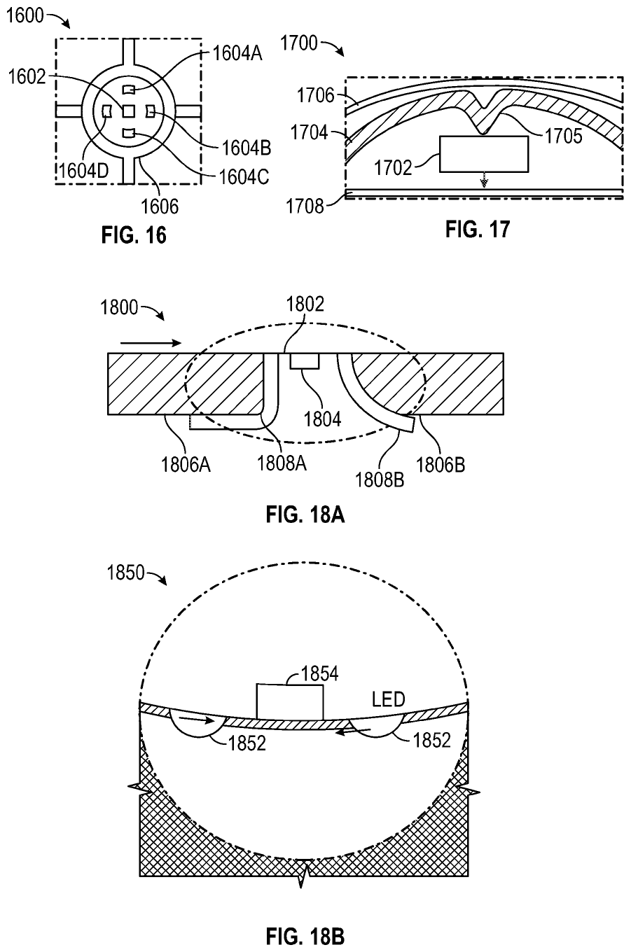

FIG. 16 illustrates a top view of an example 1600 to redirect, minimize, or eliminate sweat or other goop (e.g., dirt, oils, bacteria, or other bodily secretions or effluents) from the electronic components according to some examples. The example 1600 can include a photodiode 1602, LEDs 1604A, 1604B, 1604C, 1604D, and channels 1606. The channels 1606 can allow sweat to escape and/or evaporate. The channels 1606 can include microfluidic channels for sweat or goop that collects in troughs, indents, or tented areas or under optical sensors to escape and/or evaporate.

In some examples, the channels 1606 can be placed in between the LEDs and photodiodes. The channels 1606 can be placed in the same plane as the LEDs and photodiodes. In some examples, the LEDs, the photodiodes, and/or other components can have a coating with divots right next to the components enabling the sweat or goop to escape. In some examples, the adhesive holding the electronic device onto the skin can include divots or channels enabling the sweat or goop to escape. In some examples, the backing layer, such as the flexible PCB, can include channels built within to enable the sweat or goop to escape. In some examples, the backing layer could be a wicking and/or breathable material to help facilitate evaporation of sweat.

FIG. 17 illustrates an example 1700 with a contacting portion that applies pressure to an LED and/or a photodiode for better coupling with the skin according to some examples. The example 1700 can include a contacting portion 1704 that can include depressed portions and a protruding portion, such as the protruding portion 1705, a backing layer 1706, and an LED or photodiode 1702. The protruding portion 1705 can be in contact with the LED or photodiode 1702 at a certain contact point. In some examples, the contact portion can comprise opaque barrier material. Advantageously, the contact portion can serve the dual purpose of applying pressure and blocking light from piercing through the contact portion.

In some examples, the device is configured for the user to press down on the housing and/or other portion of the device while the device is collecting a measurement. For example, the user can initiate the determination of a physiological characteristic and/or the device can indicate to the user that the device is initiating or suggesting the user to initiate the determination of the physical characteristic (such as via an LED or a sound, or a message on an application), and the user would then subsequently hold down the device such that the sensors can get better coupling with the skin for measurement of ECG and/or PPG signals.

In some examples, the contact point can be a single point or an area on top of the LED or photodiode 1702. Advantageously, the force that the contacting portion 1704 applies to the LED or photodiode 1702 can be greater than if the backing layer 1706 was pressing against the LED or photodiode 1702 directly. The force of the contacting portion 1704 applies more targeted pressure on the contact area than if the backing layer were to be pressing down on the LED or photodiode 1702 because the backing layer is applying pressure onto the contact portion, and that pressure is being transferred onto the optical sensors on the contact point. The backing layer 1706 can include a semi-rigid backing layer that applies downward pressure indirectly on the electronic components toward the skin 1708.

In some examples, the electronic device can include wicking material near the contact area where the contacting portion presses against the LED or photodiode. Advantageously, the wicking material can prevent sweat from forming or pooling in the contact area.

FIG. 18A illustrates an example 1800 of using a physical barrier preventing adhesive or goop from overflowing into the electronics according to some examples. The example 1800 can include a backing layer 1802, an optical component 1804 (such as a photodiode), adhesives 1806A, 1806B (collectively referred to herein as adhesives 1806), and physical barriers 1808A, 1808B (collectively referred to herein as physical barriers 1808). After prolonged use of the wearable device, the adhesives 1806 can begin to leak from underneath, and could leak onto other electronic components such as the optical component 1804. Such goop (e.g., adhesive or adhesive mixed with sweat or other bodily excretions or effluents) could leak underneath an LED or photodiode blocking the light signal from being emitted or detected. The physical barriers 1808 can block the adhesive from overflowing onto electrical components, such as the LED or photodiode. The physical barriers can isolate the sensors from the adhesive or goop. In some examples, an adhesive that have a characteristic that prevent or mitigate goop from forming can be used.

In some examples, the physical barrier 1808 is tilted toward the adhesive, further reducing the amount of potential leakage of the adhesive toward the electronic components. The physical barrier 1808 also prevents the adhesive from being in contact with the electronic components, such as the LED or photodiode even before the adhesive is applied to the user. The physical barrier 1808 can include a rigid structure next to the adhesive. The physical barrier 1808 can include a thin film that is adjacent to the adhesive. The physical barrier 1808 can include a non-adhesive surface.

FIG. 18B illustrates an example 1850 of a single LED and/or multiple LEDs in the shape of a donut or ring according to some examples. The example 1850 can include a donut or ring shaped LED 1852. The donut or ring shaped LED 1852 can emit light toward the center of the donut or ring, and toward the detector 1854. In some examples, an LED can light up lighting pipes in the shape of a donut or ring. The lighting pipes can emit light in the direction of the photodiode.

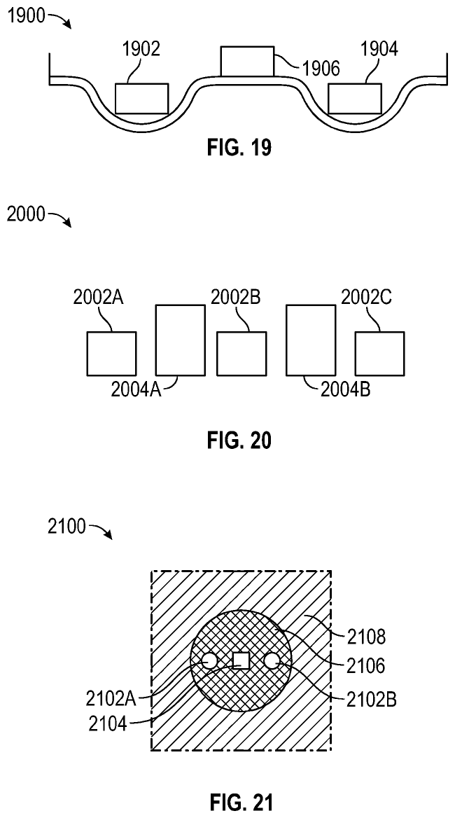

FIG. 19 illustrates an example 1900 with LEDs emitting light of different wavelengths according to some examples. The example 1900 includes an LED 1902 that can emit light of one wavelength, such as green light, and another LED 1904 that can emit light of another wavelength, such as red light. The LEDs 1902, 1904 can emit light of different wavelengths at the tented area, and the photodiode 1906 can detect light from both LEDs.

In some examples, the detector can detect light simultaneously from both LEDs, where the lights are emitted at different wavelengths. In some examples, the LEDs may emit light along multiple wavelengths or a long a spectrum focused around a particular wavelength. In some such cases, the wearable device may be configured to filter light of a first wavelength emitted from the first LED to remove of a second wavelength, which may correspond to light emitted from the second LED.

In some examples, such filtering can be via signal processing, such as a frequency filter, or a physical filter on the photodiode. For example, if green light is emitted from the left side and red light from the right side, a first photodiode can include a physical filter on the left side for green light to pass through and a second photodiode (or the same first photodiode) can include a physical filter on the right side for red light to pass through.

In some examples, the first LED can emit light and the detector can detect light from the first LED at the first wavelength, then the second LED can emit light and the detector can detect light from the second LED at the second wavelength.

FIG. 20 illustrates an example 2000 of repeating LEDs and photodiodes according to some examples. The LEDs 2004A, 2004B can emit light to the left and to the right, and the photodiodes 2002A, 2002B, 2002C can detect the emitted light. The LEDs 2004A, 2004B can emit light into tented areas between the LEDs. The LEDs can include bi-directional LEDs that emit light in two directions, such as to the left tented area and to the right tented area or omnidirectional LEDs.

FIG. 21 illustrates an example 2100 of different adhesives for the wearable device according to some examples. The example 2100 can include a photodiode 2104 and LEDs 2102A, 2102B with multiple adhesives, such as a first or inner adhesive 2106 closer to the electronic components and a second or outer adhesive 2108 on the outside.

In some examples, the first and/or second adhesive can include openings or slits enabling flexibility with patient movement. If the slits are along the vertical axis, the adhesive could stretch more horizontally if the user's movement created tension along the horizontal axis. The openings or slits can help reduce motion on the housing or on components, or can isolate the sensors from user motion.

In some examples, the different adhesives can have different levels of stickiness. For example, the first adhesive 2106 can be less sticky than the second adhesive 2108, or vice versa. The outer adhesive 2108 can be a load bearing adhesive that is thicker than the inner adhesive 2106. The thicker adhesive can flow better with the body, provide moisture, and be warmer for the skin, but may have more adhesive goop than the inner adhesive 2106.

In some examples, the inner adhesive 2106 can be thinner, optically clearer, be less sticky, have less goop forming than the outer adhesive 2108, which can be stiffer, and/or the like. In some examples, the wearable device can only have the outer adhesive 2108 without the inner adhesive 2106, or vice versa. Advantageously, the electrical components can be protected from goop escaping from the adhesives.

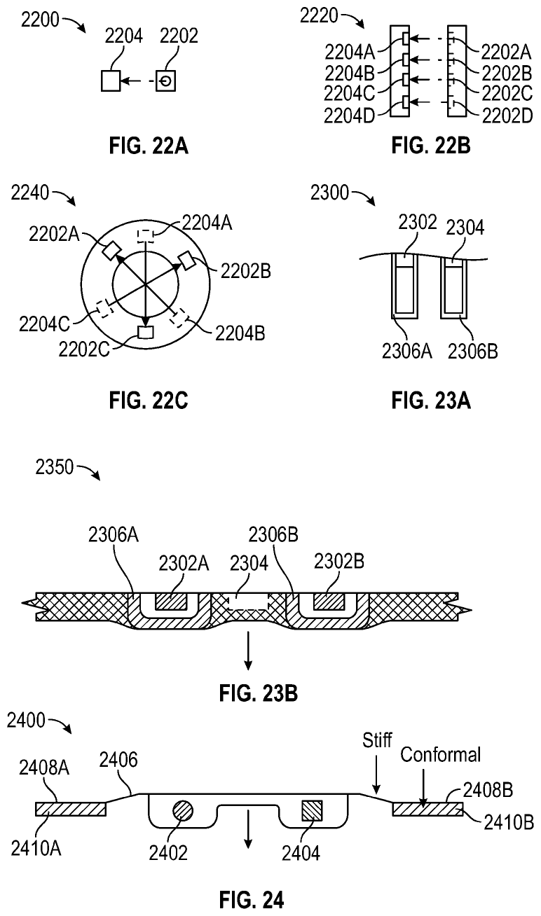

FIG. 22A illustrates an example 2200 with a single LED and a single photodiode according to some examples. The single LED 2202 can emit light toward the single photodiode 2204. FIG. 22B illustrates an example 2220 with an array of LEDs and an array of photodiodes according to some examples. The array of LEDs 2202A, 2202B, 2202C, 2202D (collectively referred to herein as array of LEDs 2202) can emit light toward the array of photodiodes 2204A, 2204B, 2204C, 2204D (collectively referred to herein as array of photodiodes 2204).

FIG. 22C illustrates an example 2240 of LEDs and photodiodes distributed in a circular array according to some examples. The LEDs 2202 can emit light toward the center of the circular array and the photodiodes 2204 can detect the light coming from the center of the circular array. The structure of the example 2240 can be such that a tented area of skin can be created within the circle for the light to penetrate. The LEDs and photodiodes can alternate around the circular array. Advantageously, any photodiode can pick up light coming from all of the LEDs that are emitting toward the same tented area of skin. The wearable device (or other computing device) can add the signals collected from the photodiode to generate a stronger signal strength. In some examples, the computing device can time align the received signal before adding the collected signals together.

FIG. 23A illustrates an example 2300 of a protrusion lined with a lighting channel according to some examples. The example 2300 includes an LED 2302, a photodiode 2304, and lighting channels 2306A, 2306B. The lighting channels or light pipes can take light emitted from the LED and channel the light through the lighting channels 2306A. The lighting channels 2306A can enable light to emit from the LED side to the photodiode side. Lighting channels 2306B on the photodiode side can capture light emitted from the LED side and pass the light to the photodiode 2304. In some embodiments, the LED and/or the photodiode include and/or are integrated with the lightning channels. Channeling the light can result in a greater percentage of the emitted light being directed from the LED 2302 to the photodiode 2304, or through the skin between the LED 2302 and the photodiode 2304.

FIG. 23B illustrates an example 2350 of using a ring or light pipe according to some examples. The example 2350 includes multiple LEDs 2302A, 2302B, a photodiode 2304, and light pipes 2306A, 2306B. Each LED can have its own light pipe to channel the light from the LED to the skin of the patient, such as to the tented area of the skin. The photodiode 2304 can capture the light emitted from multiple light pipes 2306A, 2306B, which may collectively be referred to as light pipes 2306.

FIG. 24 illustrates an example 2400 of a leaf spring pressing down on certain components of the wearable device according to some examples. The example 2400 includes a stiff portion 2406 that applies pressure to the LED 2402 and the photodiode 2404. The conformal portions of the wearable device can include a flexible portion 2408A, 2408B that may be non-adhesive and an adhesive layer on both sides 2410A, 2410B. The stiff portion can include a leaf spring to exert force downwards toward the LED and photodiode. Advantageously, the stiff portion can exert force on the LED and photodiode in order to improve coupling of the LED and photodiode with the skin. Improving coupling with the skin can include increasing contact between the LED and/or photodiode and the skin. Further, the application of pressure or force on the LED and/or the photodiode towards the skin can result in improved signal quality or be tuned to achieve better or optimal signal quality. It should be understood that the present disclosure is not limited to a leaf spring and that other types of springs or mechanisms can be used to exert force downwards toward the LED and/or photodiode.

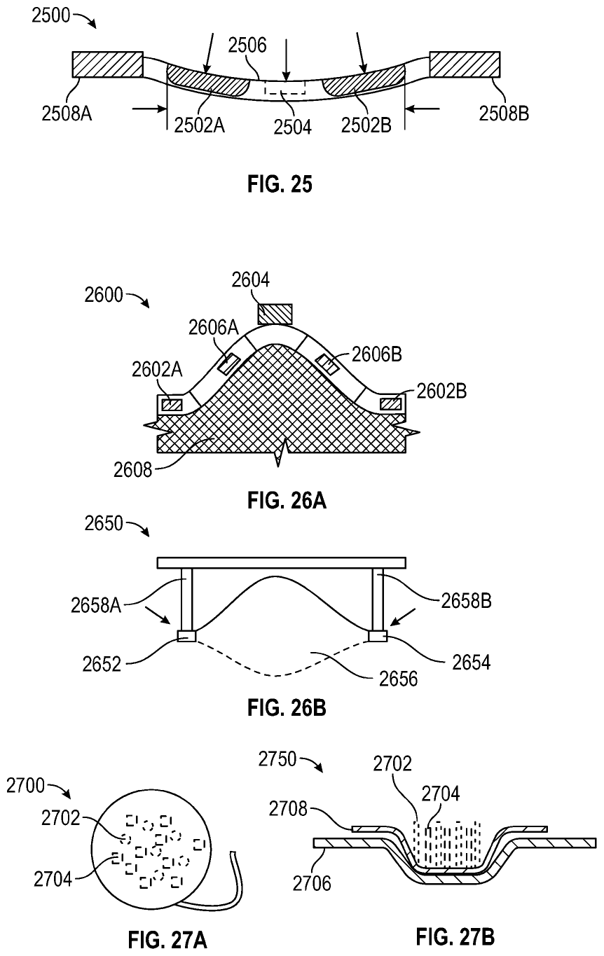

FIG. 25 illustrates an example 2500 of a bowl-shape flexible circuit that depresses into the skin according to some examples. The example 2500 can include LEDs 2502A, 2502B, a photodiode 2504, adhesives on the sides 2508A, 2508B, and a backing substrate 2506. The backing substrate 2506 can be of a bowl shape or indented inward to naturally apply pressure onto the LEDs and the photodiode to better couple with the skin. In some cases, the backing substrate 2506 is affixed to a flexible circuit and applies pressure to the flexible circuit to improve coupling of the LEDS 2502A, 2502B and the photodiode 1504 with the skin of the patient. In some examples, the backing substrate 2506 can include a protrusion that naturally applies pressure in one direction. The skin or movement of the user can apply pressure the other way, but the protrusion can be made to withstand a certain amount of pressure without losing its protrusion shape, while continuing to apply pressure toward the skin.

FIG. 26A illustrates an example 2600 of opaque barriers between the LEDs and photodiodes according to some examples. The example 2600 can include LEDs 2602A, 2602B, a photodiode 2604, and opaque barriers 2606A, 2606B, collectively referred to as opaque barriers 2606. As light is emitted from the LEDs 2602 into the tented area of the skin 2608, the opaque barriers 2606 can block the light from passing directly from the LEDs 2602 to the photodiodes 2604. In some examples, the opaque barrier 2606 is disposed closer to the LED than the photodiode.

In some examples, the LEDs can include a lens to direct light toward a certain area, such as a tented area of the skin. For example, the lens can have roughened surfaces that are not directed to the tented area of the skin, whereas the surfaces that are directed to the tented area of the skin can be clear, thus enabling light from the LED to emit toward the tented area of the skin. The roughened surfaces can create a tortuous path for light emitting toward an undesired direction.

FIG. 26B illustrates an example 2650 of pseudo-transmission directly from the LED to the photodiode through a tented area of skin according to some examples. The example includes an LED 2652 and a photodiode 2654 between a tented area of skin 2656. The skin is tented due to the exerted pressure of the LED and the photodiode onto the sides of the skin. The light signal can be emitted from the LED 2652 and pass directly through the skin to the photodiode 2654, resulting in improved signal strength over traditional reflection signals. Moreover, the LED and photodiode can be placed on elevated structures 2658A, 2658B, such as rails, to press into the skin further in order to create more tented area in the skin.

FIG. 27A illustrates a cross-sectional view 2700 of an example using a bundle of fiber optics for the LEDs and the photodiodes according to some examples. FIG. 27B illustrates a side view 2750 of the example using the bundle of fiber optics according to some examples. A first bundle of fiber optics 2702 can be attached to one or more LEDs and a second bundle of fiber optics 2704 can be attached to one or more photodiodes.

In some examples, the light emitted from the LED can emit through the first bundle of fiber optics 2702 and into the skin of the user 2706. The light can be reflected from the skin and received by a second bundle of fiber optics 2704 and transmitted back to the photodiode. The electronic device is pressing into the skin of the user 2706 to create a depression in the skin. In some examples, the fiber optics press into the skin slightly. In other examples, the fiber optics are glued to a flat surface, and the flat surface rests on the skin. In some examples, the fiber optics are separated by a certain distance, such as a centimeter, half a centimeter, quarter centimeter, and/or the like.

In some examples, the LED and photodetector can share the same fiberoptic channel. For example, the bundle of fiber optics can be braided into the same fiber optic channel, such that a single bundle of channels can separate out of one end to connect with the LED and photodetector, and the other end onto a layer 2708 that contacts the skin. In some examples, the fiberoptic channel can have an inner core for the LED and an outer core for the photodetector.

In some examples, the same channel is used for the LED and photodetector. For example, the fiberoptic channel is used for the LED when the LED is emitting light, and a switch switches the fiber optic channel to the photodetector when the photodetector is detecting light.

In some examples, the electronic device includes an accelerometer to account for motion. For example, the electronic device can discard readings if high motion is detected. The electronic device can also remove noise cause from the motion, such as if the motion is of a certain frequency.

In some examples, a specific input impulse signal or signal signature can be transmitted to the LED. At the photodetector, emitted light is detected, and the processor looks for the specific input impulse signal or signal signature that was inputted into the LED. Applying such a filter, such as a match filter, can significantly boost the signal-to-noise ratio of the received signal.

In some examples, the ECG and PPG sensors are placed far apart, such as one on the chest and another on a finger or a toe. The received ECG and PPG sensor data can be received by the same circuit which can make time syncing easier. The circuit can estimate what the time delay is to provide information on the blood pressure, as further disclosed herein. The signals travel fast enough such that the time delay will not be larger than the length of a pulse.

### Skin Coupling Examples

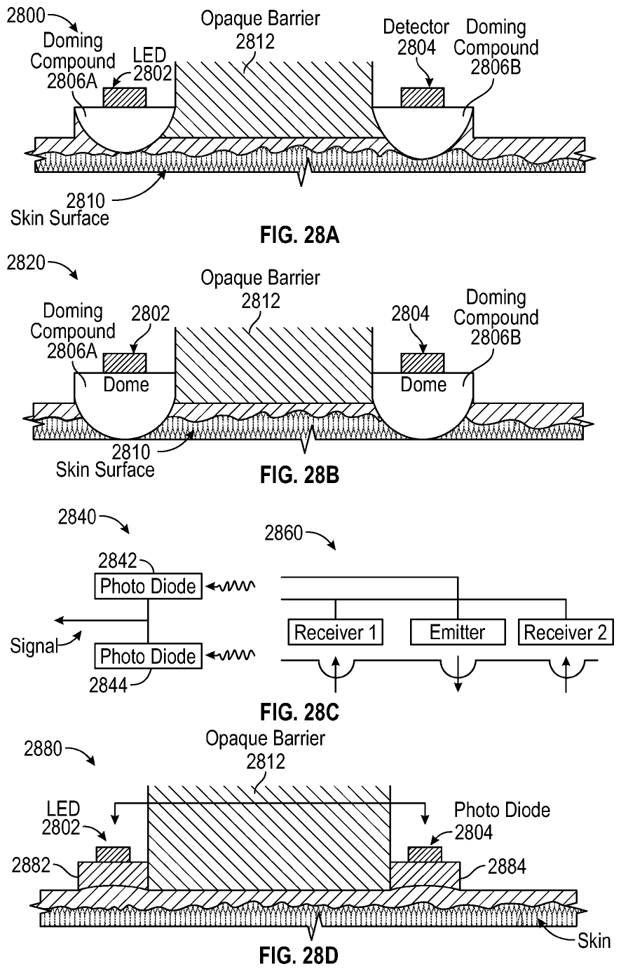

FIG. 28A illustrates an example of skin coupling via two doming compounds according to some examples. For example, the skin coupling may be between the device 1200 and the chest of a patient. Moreover, the skin coupling may be between flexible wings (e.g., wing 330 or wing 331) of the device (e.g., device 300) and the chest of the patient. The skin coupling example 2800 can include an LED 2802, a detector 2804, two doming compounds 2806A, 2806B (collectively referred to herein as doming compounds 2806), and/or an opaque barrier 2812. The doming compound 2806 can be in a shape of a half sphere shape. The dome compounds 2806 can extend from the LED 2802 and the detector 2804 into the skin surface 2810 of a patient. The doming compound 2806 can include a compound such as a glass lens to transmit an optical signal. The doming compound 2806A can be in the shape of a dome or a convex lens to direct light from the LED 2802 into the skin and/or the doming compound 2806B can be in the shape of a dome to direct light from the skin back to the detector 2804.

In some examples, the skin coupling example 2800 includes an opaque barrier 2812. One of the possible disadvantages of having an LED 2802 and a detector 2804 close together is potential cross-talk of the optical signal, where at least a portion of the optical signal goes directly from the LED 2802 to the detector 2804 without passing through the skin. With cross-talk signals, the detector 2804 is not detecting a signal that contains any physiological information. The opaque barrier 2812 can be included to block the signal from going directly from the LED 2802 to the detector 2804.

Although the skin coupling example 2800 includes an opaque barrier 2812, it is appreciated that other barriers that block optical signals can be used. For example, one or more barriers that absorb stray light rays and/or reflect these rays can be applied to limit light from directly traveling from the LED to the photodetector. The one or more barriers can be configured to funnel and/or direct oblique light rays into the photodetector. In some examples, the opaque barrier 2812 can extend over beyond the LED 2802 and/or the detector 2804. In other examples, the opaque barrier 2812 can partially extend beyond an edge of the LED 2802 and/or the detector 2804 closest to the opaque barrier. For example, the dome can include a circular and/or rounded portion that bends inward away from the opaque layer, and the opaque layer can fill in a gap that would otherwise not be filled in by a rectangular shaped opaque barrier. In some examples, the opaque barrier 2812 can be of varying thicknesses and/or heights. In some examples, the opaque barrier 2812 can be of a certain height that is lower than the top of the dome, such that the opaque barrier comes up to a different point of the dome.

In some embodiments, the device can block an undesired signal or undesired frequencies via the doming compound having certain light refracting or optical beamforming properties. The doming compound may direct or redirect light away from the other optical component without a physical barrier corresponding to particular frequencies.

In some embodiments, the device can block undesired signal using certain substances, such as a substance that is silicone doped with a black dye to create the physical barrier. The substance can include a type of biocompatible epoxy (e.g., flexible after cure) that can be opaque or made opaque via dye or other process.

In some embodiments, the device can block undesired signal based on a thickness of material in the z-dimension of the opaque barrier. The opaque barrier could be at least at the height of the LED and PD, but could also extend beyond the LED and/or the PD. If there's a doming compound, the opaque barrier can be at the height of the dome (such as the embodiment shown in FIG. 28A), or a height along the dome. The opaque barrier doesn't need to span/bridge the space between the LED and PD. The dome could include a wire (e.g., an electrically-insulating material) being laid down between the LED and PD.

In some examples, the skin coupling example can include a reflective layer. For example, the reflective layer can reside next to the opaque layer. The portion of the opaque barrier 2812 that could come in contact with the skin could have a reflective layer in between.

Advantageously, the light transmitted from the doming compound 2806A could be reflected off of the reflective barrier instead of being absorbed by the opaque barrier 2812. Reflecting light from the reflective barrier can improve the signal quality as the light travels from the doming compound 2806A to the doming compound 2806B.

FIG. 28B illustrates another example of skin coupling via two doming compounds according to some examples. In the skin coupling example 2820, the two doming compounds 2806 can be placed lower such that the doming compounds press further into the skin surface. In some examples, the two doming compounds 2806 can be of a greater length such that the doming compounds press further into the skin surface. In the skin coupling example 2800, the doming compounds 2806 can include a curved surface, such as a dome shape. The amount of indent the doming compounds press into the skin surface can depend on a variety of factors. For example, the amount of indent can depend on whether the indent could be blocking smaller blood vessels close to the surface to preclude these signals from being processed. The amount of indent could depend at least partially based on the amount of light and associated hemodynamic information via interaction with the blood vessels that can be transmitted from the LED to the detector, which could lead to a larger and/or more accurate signal.

In some examples, the curved surface can curve for a certain degree amount, such as 180 degrees. At least a portion of the curved surface can be configured to contact the skin surface 2810 of the patient. For example, in the skin coupling example 2800, a portion of the curved surface contacts the skin surface 2810 and another portion of the curved surface contacts the opaque barrier 2812. Advantageously, sweat could be trapped in between the doming compound and the opaque barrier and/or the opposite side of the doming compound. Other substances, such as sweat, can be helpful in coupling light coming in and out of the skin.

In another example, the skin coupling example 2820 includes a curved surface where the entire curved surface is configured to contact the skin surface of the patient. One side of the cylindrical portion of the doming compound 2806 is in contact with the opaque barrier 2812. In this example, the cylindrical portion of the doming compound 2806 is flush with the opaque barrier 2812. Advantageously, other substances, such as sweat of the user, can be prevented from becoming trapped between the doming compounds 2806 and the opaque barrier 2812, if other substances are undesirable such as for cleanliness (such as for devices left on the patient for many days) or if the substance disperses the signal instead of focusing the light onto the skin. Other substances, such as sweat or grime could have an effect on index matching. For example, the light can be deflected as it enters and/or exits the substance as a result of mismatching indices of refraction between two materials. The index of refraction of the substance can be desirable or undesirable based on the direction of the light from the LED to the skin or from the skin to the detector. Typically, a substance like sweat that has a similar index of refraction to skin will help match the interface and limit back-reflection from the skin surface that would result from a large mismatch.

In the skin coupling example 2820, the doming compound 2806 can press into the skin more than the skin coupling example 2800. Advantageously, the skin coupling example 2820 can lead to more transmission-mode PPG type signals that can travel directly from a curved surface of the doming compound 2806A for the LED 2802 into the skin surface and through to the doming compound 2806B for the detector 2804. In contrast, the skin coupling example 2800 could capture more reflection-mode PPG type signals that bounce back from tissue components in the patient's body, such as the blood vessels. In skin coupling example 2820, transmission-mode PPG signals can be used to capture more of the signal from superficial blood vessels. However, reflection-mode and/or transmission-mode PPG signals can be used to detect more of the deeper blood vessels. In some examples, both transmission signals and reflective signals can be present and can be used to generate a more accurate optical signal and/or one that provides more information about different layers of skin tissue.

In some examples, the skin coupling examples can include an optically clear film or adhesive that can include wavelength guiding properties that helps redirect light to travel more effectively from the LED to the detector. The wave guiding properties can include polarized lenses that can allow light to pass in a particular direction, such as more from the LED toward the skin surface in the direction of the detector. Advantageously, less light can be lost in the other direction of the LED, away from the detector. In some examples, other directional light emitting technology can be used, such as phased-array optics that control phase and amplitude of light waves transmitted, reflected, or captured by a surface. Such adjusting of the phase and/or amplitude can be applied to steer the direction of light beams in a particular direction without any moving parts. Advantageously, the light beam can be steered in a direction from the LED to improve a stronger signal detection at the detector. Moreover, on the receive side, the detector can include a phase-array optics detector that can retrieve a stronger signal from the LED.

FIG. 28C illustrates an example of circuits 2840, 2860 according to some examples. Circuits 2840, 2860 can include two photo diodes 2842, 2844. In some examples, the system can include multiple LEDs or receivers. The circuits include two receivers and one emitter. The system can subtract or reduce noise coming from the circuits by rejecting common baseline information present on both receivers that can include electrical noise such as 60 Hz noise. In some examples, one photodiode can act as a reference signal for the amount of optical power launched into the skin with another photodiode acting as the receiver diode. If the bandwidth of the power reference photodiode op-amp circuit is the same as that of the receiver photodiode, then the power fluctuations in the LED output can be canceled up to about ⅓ of the bandwidth by comparing the reference photodiode signal to the receiver photodiode via a technique called autobalanced detection. Autobalanced detection can be very effective at cancelling common mode noise to very high order, for example if the LED output is fluctuating in a manner that is impacting the extraction of the detector signal The system can also increase the signal-to-noise ratio (SNR) of the signal by adding together AC components of signal on both receivers that capture hemodynamic information from light interacting with blood vessels on both sides of the single emitter, increasing the area of skin sampled. By using different geometric configurations of the receivers and emitters, different areas of the skin can be sampled. The two receivers can also be placed at different distances from the emitter, which would allow the signal to interact with different tissue depths and provide information about both superficial as well as deeper blood vessels. The two receivers can also contain different wavelength filters to capture more complex information about how different wavelengths of light interact with vascularized tissue.

FIG. 28D illustrates a skin coupling example using an inward meniscus or concave shape for the glass lens according to some examples. The skin coupling example 2880 can include an LED 2802, a detector 2804, an opaque barrier 2812, and inward meniscus glass lenses 2882, 2884. The detector 2804 can be a photodiode or any other type of light detector. The inward meniscus glass lenses 2882, 2884 can intentionally trap other substances, such as saline or sweat, which can have index matching to the stratum corneum, the top layer of skin. Advantageously, the meniscus design can enable sufficient moisture on the skin to improve light entering into the skin. Further, the concave lens can tent skin towards the LED 2802 and the detector 2804 (e.g., photodiode) when the device (e.g., the device 1200 or the device 300) is affixed to the skin (e.g., the chest) of the user.

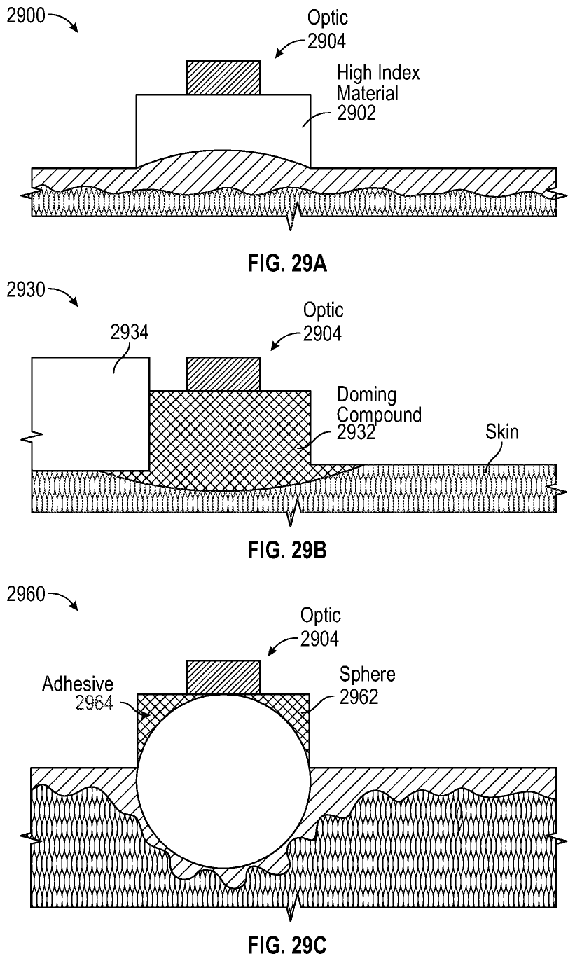

FIG. 29A illustrates the use of a high index material according to some examples, such as the skin coupling example 2900. For example, the material may have a refractive index of greater than 1.5, greater than 1.6, between 1.55 and 1.75, greater than 1.75, greater than 1.8, and the like. The high index material 2902 can include a meniscus shape, and can be index-matched to the stratum corneum. For example, the high index material may be selected to have the same refractive index as the stratum corneum. The high index material 2902 can be disposed below the optic 2904 and may be used, for example, to form the domes represented by the doming compounds 2806 or the meniscus or concave shaped lenses 2882, 2884. The optic 2904 can include the optical component such as an LED or a photodiode. The high index material 2902 can include the inward meniscus glass lens 2882 or 2884 in FIG. 28D.

FIG. 29B illustrates a doming compound that protrudes past the opaque barrier 2934 overlapping at least a portion of the opaque barrier 2934 in a lateral direction and increasing the area of contact with the skin, according to some examples. The skin coupling example 2930 includes a doming compound 2932 that protrudes beyond where the opaque barrier begins. Advantageously, sweat cannot be trapped in between the doming compound and the opaque barrier. Moreover, the shape of this doming compound could allow light to be emitted underneath the opaque barrier, increasing the amount of light able to interact with the skin and be received by the detector.

FIG. 29C illustrates a ball lens example according to some embodiments. In some examples, the skin coupling example 2960 can include a full circle, ball, or sphere 2962. The sphere 2962 can include a sapphire ball lens, which has the advantage of good index matching to the skin. The adhesive 2964 used to attach an LED or detector to the sphere 2962 can be applied to at least a portion of the curved surface of the sphere 2962.

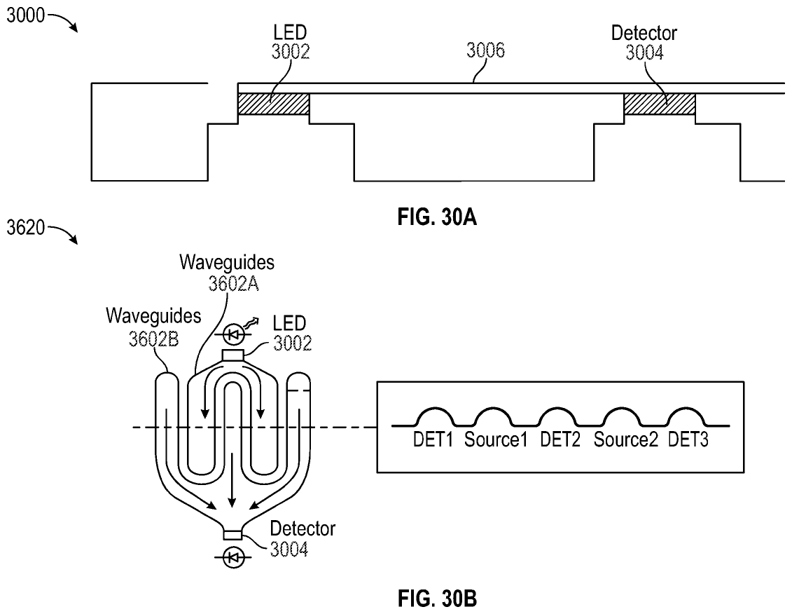

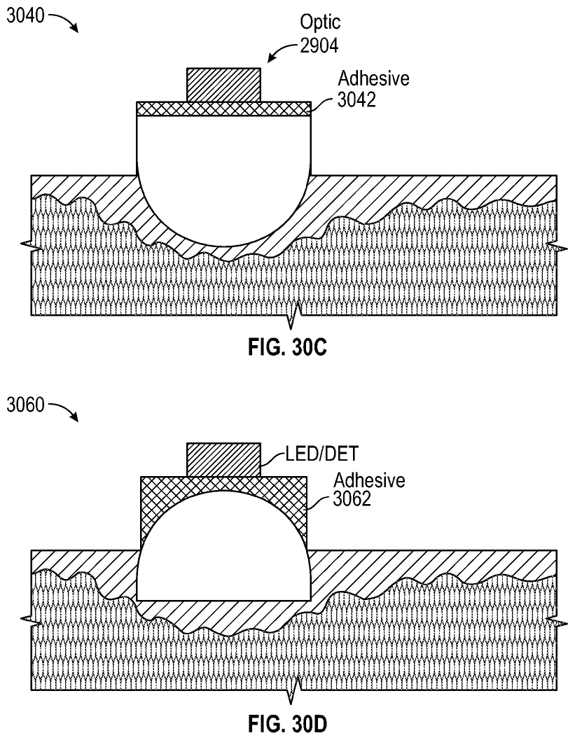

FIG. 30A illustrates the basic template of an LED and detector mounted on a flex circuit, from which waveguides could be built on the surface according to some examples. This example 3000 can include an LED 3002, a detector 3004, and flexible circuits 3006. The flexible circuit 3006 can include a surface portion that can couple with the skin of a patient.

FIG. 30B illustrates one example of a waveguide flexible circuit 3620, according to some examples. The waveguide 3602A can be attached to the LED 3002 such that the light emitting from the LED 3002 can flow throughout the waveguide 3602A. The detector 3004 can include a waveguide 3602B whereby light can be captured through various areas and/or throughout the waveguide 3602B and received at the detector 3004. The waveguides can be made of an adhesive polymer that is attached to the polyimide backing of the flex circuit holding the LED and detector.

In some examples, the flexible circuits can include a 2D spread that tracks across a wider area of the skin portion, such as across a wider portion of a user's chest. Using these waveguides, the system can include a smaller number of LEDs and/or detectors, such as 1 LED and 1 detector in FIG. 30B, but have multiple sources of data, such as effectively having 3 detectors and 2 LED light sources (DET 1-SOURCE 1-DET 2-SOURCE 2-DET 3). Moreover, light that is passed from Source 1 to detector 1 may have a larger signal but may penetrate the skin more shallowly than a signal that is emitted from Source 1 to detector 3. Signals transmitted from various sources along the LED waveguide 3602A and/or received from various tracks along the detector waveguide 3602B can be combined to create a stronger detection signal or analyzed by separation distance to obtain more detailed information related to tissue depth.

FIG. 30C illustrates an example of adhesives used for a dome shape glass lens, according to some examples. The example 3040 includes an adhesive 3042 that attaches to the LED/detector and the dome shape glass lens. FIG. 30D illustrates an example of adhesive used for an inverse dome shape glass lens, according to some examples. The example 3060 includes an adhesive 3062 that attaches to the LED/detector and the inverse dome shape glass lens. An inverse dome shape lens could have the advantage of a flat surface of contact to limit blanching of the skin. The LED and and/or detector is glued to the glass lens via the adhesive. The adhesive can be an assembly including an optically clear film, an optically clear adhesive, or a high index resin or epoxy. The opaque barrier can then be backfilled in the surrounding areas.

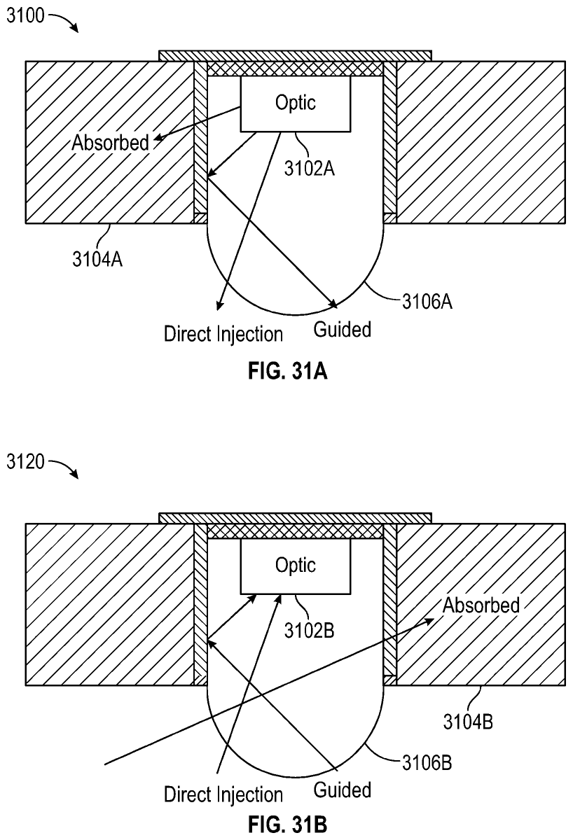

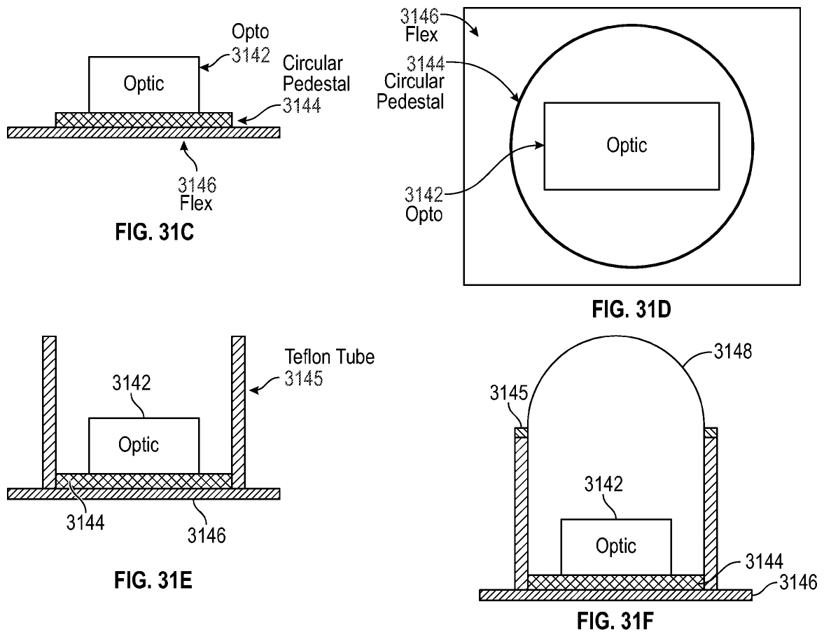

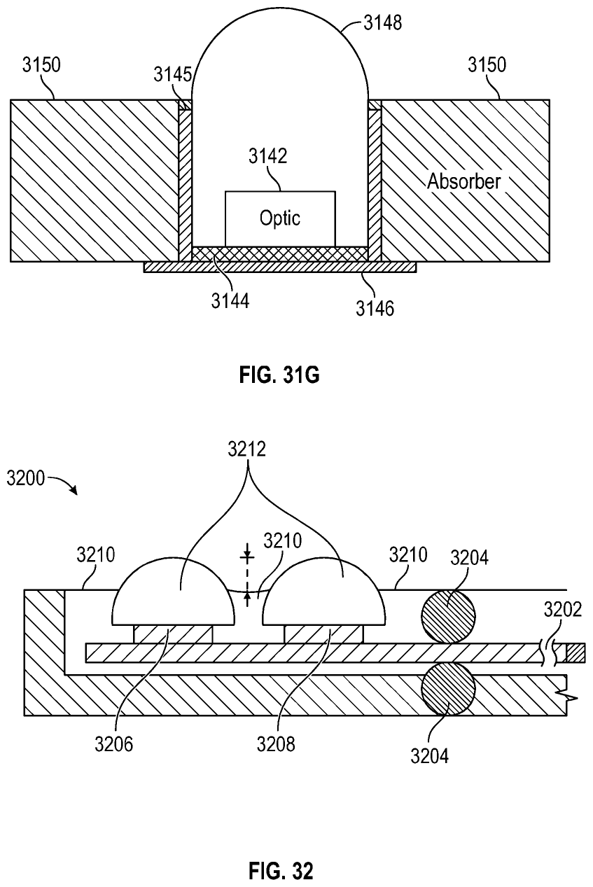

FIG. 31A illustrates an example of ray tracing of LED emitted light, according to some examples. FIG. 31B illustrates an example of ray tracing of detected light, according to some examples. In both examples 3100, 3120, light can either be absorbed by an opaque barrier 3104A, 3104B, bounced off a wall and guided, or directly injected. In the example 3100, light can be emitted from the LED 3102A into the glass lens 3106A. The light can be absorbed by the opaque barrier 3104A, and/or penetrate the skin by being guided by bouncing off a surface and/or directly injected into the skin.

Light that has been emitted from an LED, such as LED 3102A, and emitted from a glass lens, such as 3106A, into the skin can scatter, reflect, and be absorbed by the patient's body. The reflected light can be received on the detector side. In the example 3120, the light can be received into the glass lens 3106B and absorbed by the opaque barrier 3104B, and/or be received by a detector 3102B by being guided by bouncing off a surface and/or directly injected into the detector 3102B. Advantageously, if the LED and detector are placed on the surface of the chest, the LED can transmit light into the chest of the patient, some light can reflect back after interacting with a lot of surface capillaries that are feeding and/or vascularizing the top layer of skin, and the detector can detect these signals. These signals can be used to determine blood pressure, and other hemodynamic characteristics.

FIGS. 31C, 31D, 31E, 31F, and 31G illustrate steps for creating different types of domes over the LED and detector, according to some examples. FIG. 31C illustrates an example of a flexible board 3146, a circular pedestal 3144, and an LED/detector 3142, according to some examples. FIG. 31D illustrates a top view of FIG. 31C, according to some examples. The circular pedestal 3144 can be placed on top of a flexible board 3146. The LED/detector 3142 can be placed on top of the circular pedestal 3144.

FIG. 31E illustrates an example of adding a Teflon tube 3145, or tube of other material, according to some examples. The Teflon tube 3145 can absorb and/or reflect signals coming into the detector and/or emitting from the LED. FIG. 31F illustrates an example of adding or creating a lens 3148 to fit within the Teflon tube 3145 and on the LED/detector 3142, according to some examples. The lens 3148 can be made by filling the mold with UV cure or other optically correct adhesive, and the desired meniscus can be formed. The lens 3148 can scatter and/or direct light from the LED or to the detector 3142. Using this method, the lens 3148 can be created to be thicker or thinner, such as depending on the desired indentation on the patient's skin. FIG. 31G illustrates the opaque barrier 3150 being added to the sides of the Teflon tube 3145, according to some examples. The opaque barrier 3150 can prevent and/or mitigate cross-talk between the LED and the detector.

FIG. 32 illustrates a mold to create the opaque barrier surrounding the sapphire half ball glass lenses, according to some examples. In this example 3200, the flexible optics board 3202 can include a flexible polyimide substrate. A photodiode (e.g., the detector 3206) can be soldered onto the flexible optics board 3202. an LED (e.g., LED 3208) can be soldered onto the flexible optics board 3202. The sapphire half ball glass lenses 3212 can be glued onto the detector 3206 and the LED 3208. A blocker, such as an O-ring 3204 can be placed around the flexible optics board 3202, so that when the material used to create the opaque barrier 3210 is injected or otherwise applied around the areas of the LED, detector, and the half-ball lenses, the opaque barrier 3210 is blocked from simply flowing out on the right side. The amount of opaque barrier 3210 can be filled in based on how much of the glass node is desired to be exposed and/or to contact the skin of a patient.

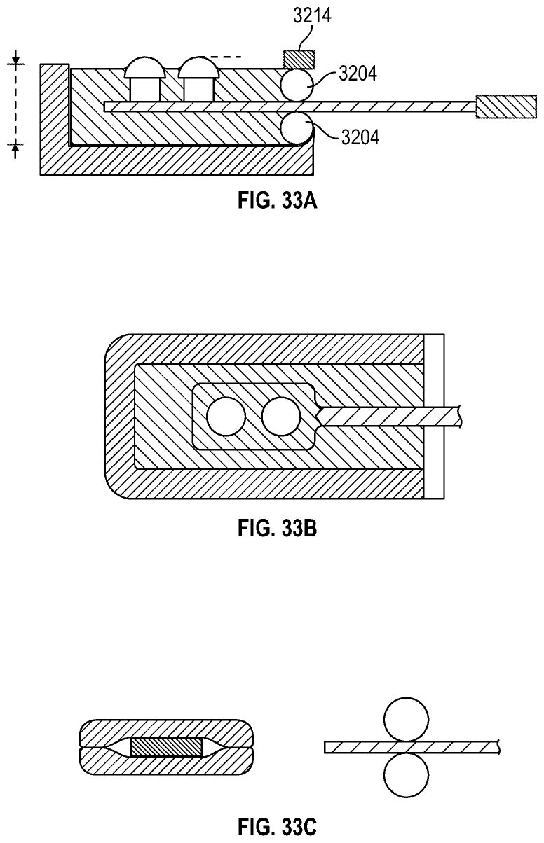

FIG. 33A illustrates a clamp 3214 that can clamp down the O-ring 3204, according to some examples. FIG. 33B illustrates a top view of FIG. 33A, according to some examples. FIG. 33C illustrates a side and cross-sectional view of the O-ring 3204, according to some examples.

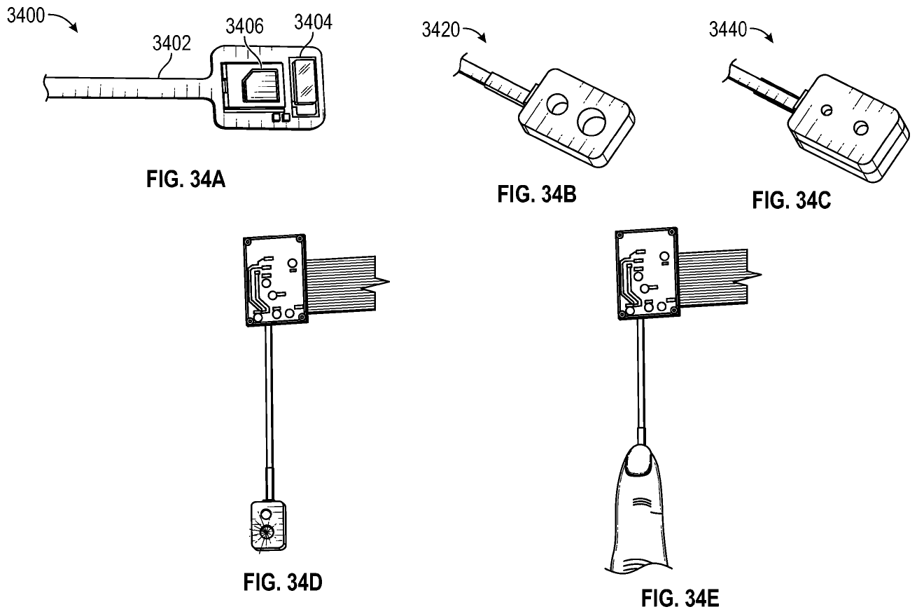

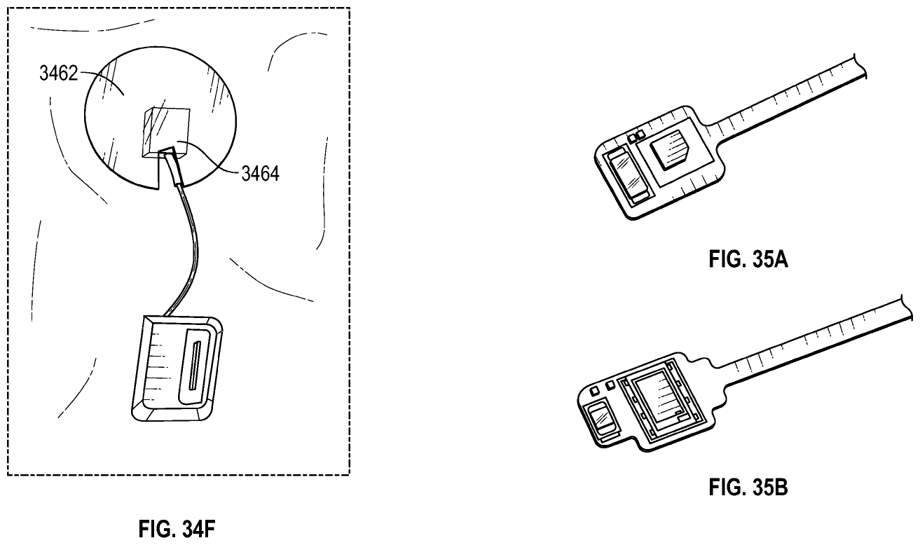

FIGS. 34A, 34B, and 34C illustrate a prototype of a skin coupling example, according to some examples. The example 3400 is an example flexible optics board that can include a flexible printed circuit (FPC) cable 3402, an LED 3404, and a detector 3406. FIGS. 34B and 34C illustrate examples of different amounts of the optical barrier applied, according to some examples. The example 3420 of FIG. 34B illustrates a smaller amount of the optical barrier applied than the example 3440 of FIG. 34C, as the optical barrier for FIG. 34B is of a thinner thickness and more of the LED and detectors are exposed.

FIGS. 34D and 34E illustrate the skin coupler in action, according to some examples. FIG. 34D illustrates light emitting from the LED and FIG. 34E illustrates a user's finger over the LED. The detector is on the same side as the LED and detects signals emitted from the LED and that pass through the body and reflect back to the detector.

FIG. 34F illustrates another example of a skin coupler where the flexible optics board can be placed directly on the surface of the chest and held in place using an adhesive 3462 to hold the housing 3464 that includes the optics onto the skin of a patient.

FIGS. 35A and 35B illustrate an example of a skin coupling example using conformal coating, according to some examples. FIG. 35A illustrate the use of conformal coating of a certain thickness, and FIG. 35B illustrate the use of conformal coating of another thickness. However, it is appreciated that other thicknesses can be applied, such as 10, 20, 50, 100, 200, 500, 1000 um thickness. The LED and photodiode protrude from the flexible optic layer, and thus a conformal coating over the LED and photodiode portion can electrically insulate the user between the optics and their skin, and provide mechanical protection to soften any sharp edges of the optics. With a thin coating, the LED and photodiode can still indent the skin of the patient, such as skin on the chest of the patient.

The conformal coating can include an optically clear substance, an electrically insulating substance, and/or a high finish epoxy that is biocompatible. Advantageously, the conformal coating approach enables the use of a much thinner substance, enabling a much thinner form factor. Advantageously, a thinner form factor can enable better coupling of an optical component to the skin, for example by integrating it seamlessly with an adhesive assembly. A thinner form factor may also require less force to apply adequate pressure to the optical component to achieve sufficient coupling to the skin. Furthermore, with the thinner conformal coating, in the micrometer thickness range (e.g., 100-200 micrometer thickness), crosstalk between the LED and the detector is greatly reduced. In contrast to using a large glass lens, the conformal coating does not direct light (or significantly reduces the amount of light directed) from the LED to the detector. If the conformal coating is thin enough, with the wavelength of light and how it travels horizontally, the thin layer of conformal coating prevents (or significantly reduces) the amount of cross-talk between the LED and the detector. Since the conformal coating is thin, the light can be reflecting so much so that more of the light ultimately penetrates the skin instead of reflecting back again to reach the detector directly without physiological contact. In contrast, if the conformal coating is thick, there is a higher probability of light bouncing off and traveling to the other end of the conformal coating (such as right next to the detector).

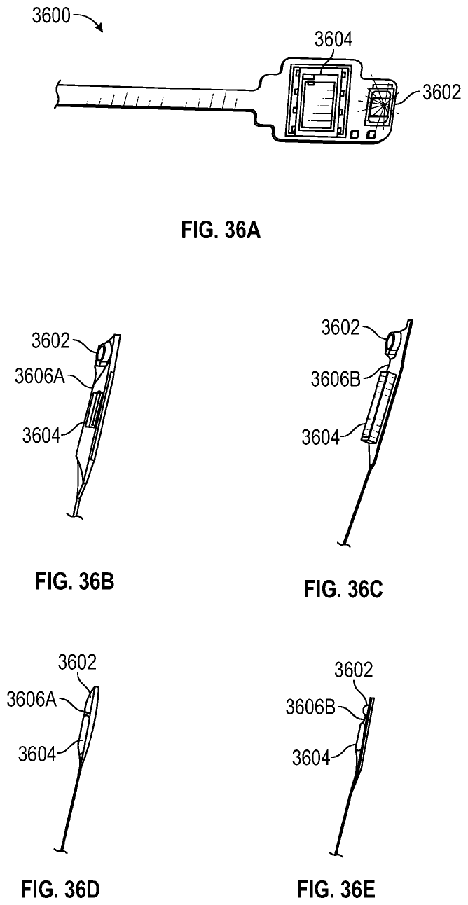

FIG. 36A illustrates an example 3600 of how light can be emitted from the emitter 3602 onto the skin of a patient, according to some examples. FIGS. 36B and 36D illustrate examples of a light emitting diode (LED) 3602, a detector 3604, and a bridge 3606A of epoxy in between according to some examples. The bridges 3606A in these examples are thicker than the bridges 3606B shown in FIGS. 36C and 36E. The bridges 3606B in FIGS. 36C and 36E illustrate an example where more of the epoxy in the bridge between the LED 3602 and detector 3604 is removed, further reducing the possibility of cross-talk between the LED 3602 and the detector 3604.

Advantageously, conformal coating over all components helps to protect the patient by applying the conformal coating over any exposed solder or exposed electrical contact pads. Moreover, the epoxy is biocompatible and a single surface layer as opposed to multiple layers (such as having an opaque barrier and glass lens). Moreover having the LED directly emit light onto the skin through a thinner lens can provide a more concentrated light than using a thicker or domed glass lens that could disperse the light onto the skin and reduce the amount of light that has the opportunity to interact with the tissue of interest and reflect back to the detector.

In some examples, biocompatible substrates, LEDs and/or detectors can be used such that the conformal coating may not need to cover the entire LED, photodiode and/or substrate surface, or require a thinner coating.

In some examples, the conformal coating helps to even the thickness of the device around the LED and photodiode. For example, the LED and photodiode may be of different thicknesses, the conformal coating may be applied such that the thickness of the LED and the photodiode are closer than if the conformal coating was not applied. Advantageously, when placing the device on the body of a patient, having a smaller varying thickness can help to obviate or mitigate air gaps forming between the LED/detector and the skin of the patient.

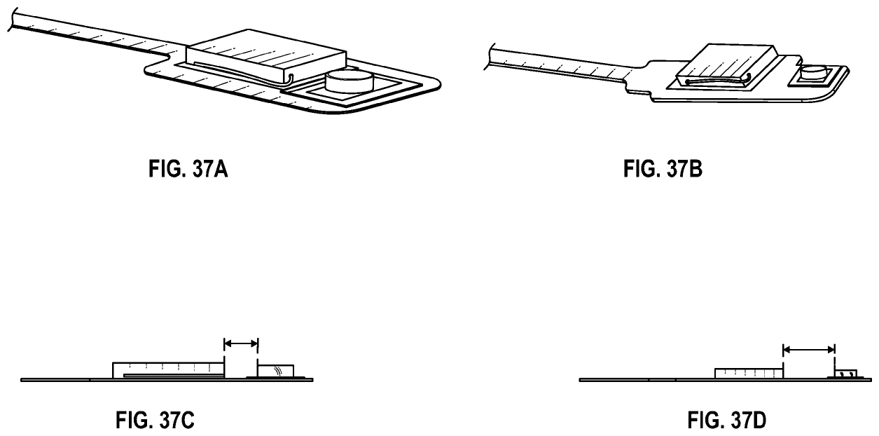

FIGS. 37A and 37C illustrate a first example of a skin coupling example, and FIGS. 37B and 37D illustrate a second example of a skin coupling example. FIGS. 37A and 37C can have different components than FIGS. 37B and 37D, such as a different LED or photodiode. The spacings between the photodiode and LED for FIGS. 37A and 37C is smaller than the spacings for FIGS. 37B and 37D. For example, the spacing between the photodiode and the LED can be 1 micrometer, 2 micrometer, 5 micrometer, 10 micrometer, 20 micrometer, 30 micrometer, 40 micrometer, 50 micrometer, 100 micrometer, 200 micrometer, 500 micrometer, 1 millimeter, 1.5 millimeter, 2 millimeter, 2.5 millimeter, 5 millimeter, 10 millimeter, spacings between the above distances, etc. The spacing can be from the center of the LED to the edge of the photodiode. The LED can be a red LED, green LED, infrared LED, and/or the like. The LED can emit light at a particular wavelength and/or varying wavelengths. The photodiode can measure light at a particular wavelength and/or varying wavelengths.

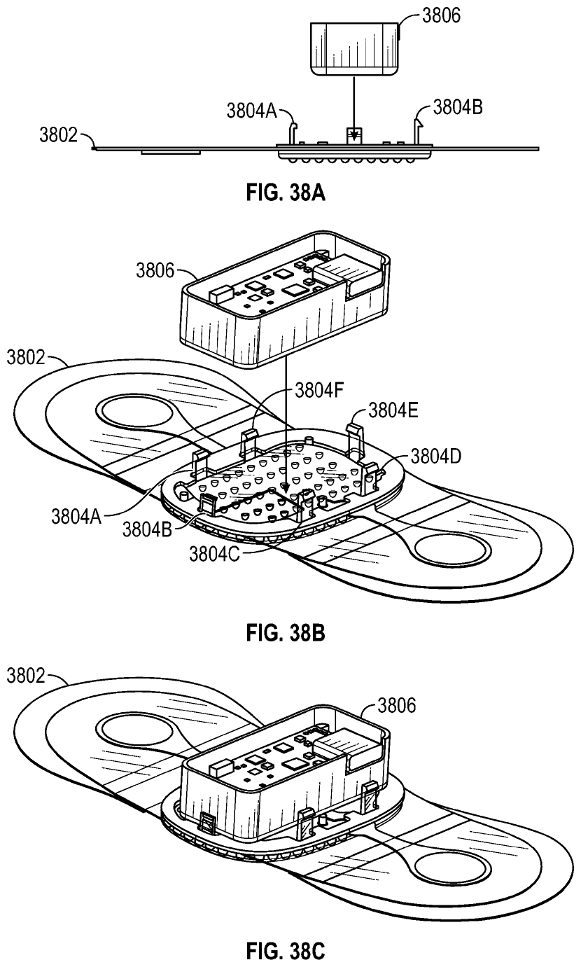

FIGS. 38A-40B illustrate examples of an adhesive assembly mating with a housing surrounding the electronics, according to some examples. The adhesive assembly can include portions of the wearable device that adheres to the skin of the patient as illustrated and described for example with respect to FIGS. 14A and 14B and well as elsewhere herein. The adhesive assembly 3802 can include mating components, such as 3804A, 3804B, 3804C, 3804D, 3804E, 3804F (collectively referred to herein as mating components 3804). FIG. 38A illustrates a front view and FIG. 38B illustrates a perspective view of the electronic component 3806 over the adhesive assembly 3802. FIG. 38C illustrates a perspective view of the electronic component 3806 mating with the adhesive assembly 3802.

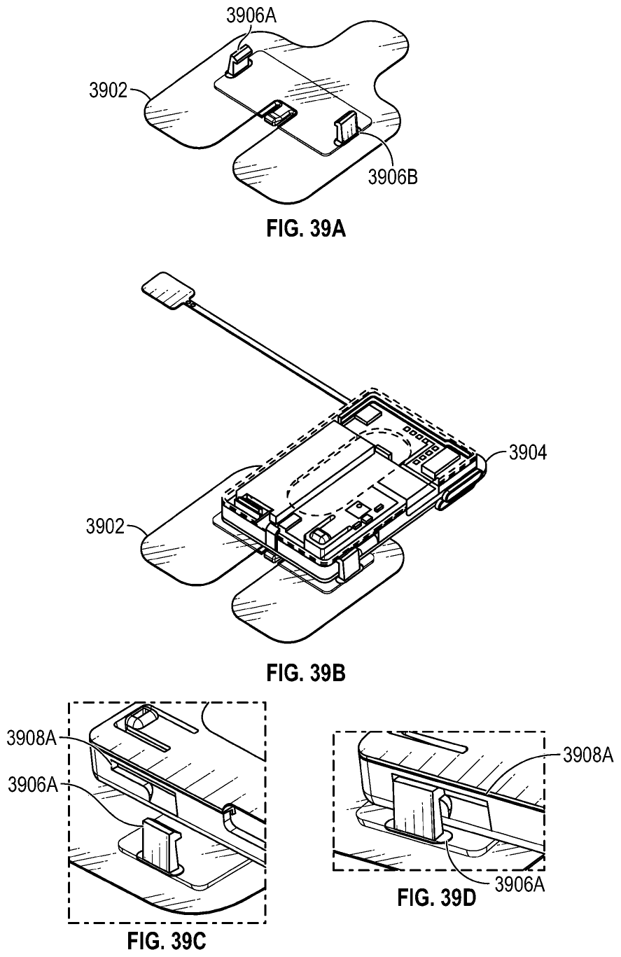

FIGS. 39A-39D illustrate another example of an adhesive assembly 3902 mating with an electronic housing 3904, according to some examples. The adhesive assembly 3902 can include a first mating portion 3906A, 3906B. The first mating portion 3906A can be configured to mate with a second mating portion 3908A on the electronic housing 3904. In some examples, there is a single mating portion on the electronic housing 3904 and/or the adhesive assembly 3902. In some examples, there is a plurality of first mating portions on the adhesive assembly 3902 and/or a plurality of second mating portions on the electronic housing 3904. Advantageously, the adhesive can be left on the body and the electronic housing can be swapped for a multitude of reasons, such as for ease of application, switching out the battery, downloading data from the patch, and/or the like. Moreover alternatively, adhesives can be swapped in and out while still using the same electronics.

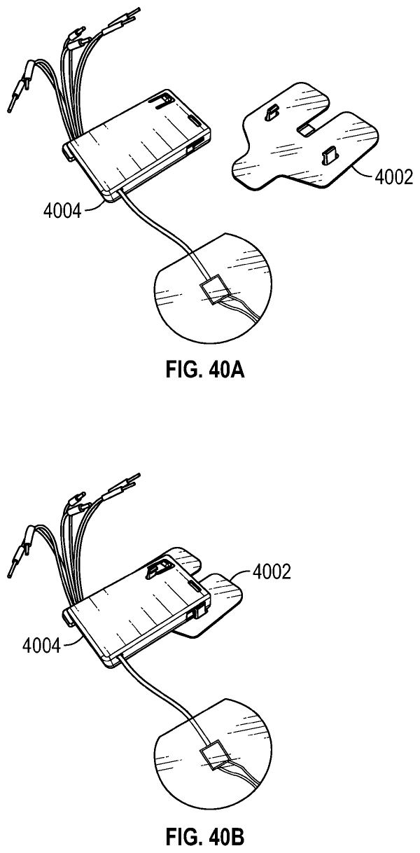

FIGS. 40A and 40B illustrate an example of an electronic housing mating with an adhesive assembly, according to some examples. For example, FIGS. 40A and/or 40B can include one or more examples of FIGS. 39A-39D with a top covering to the housing. The electronic housing 4004 can snap onto certain fixtures of the adhesive assembly 4002. The electronic housing 4004 can include the LED and/or the detector for emitting and/or detecting light off of the skin of the patient.

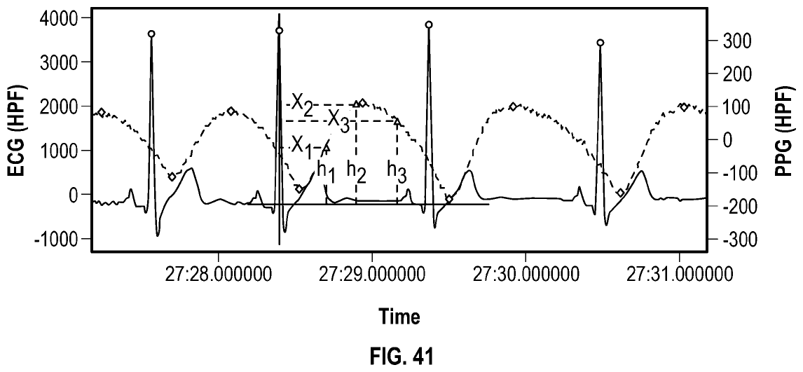

FIG. 41 illustrates a graph of ECG and PPG signals to determine PAT according to some examples. PAT can be calculated as an integral according to the following equation:

$${PAT}_{integral} = \frac{{x}_{1} {h}_{1} + {x}_{2} {h}_{2} + {x}_{3} {h}_{3} + … + {x}_{n} {h}_{n}}{{h}_{1} + {h}_{2} + {h}_{3} + … + {h}_{n}}$$

In some embodiments, PAT can be calculated using ECG R-peak to PPG peak information and/or using ECG R-peak to PPG foot/valley information. In other embodiments, PAT can be calculated using an integral of the PPG signal by weighing each new sample by its amplitude.

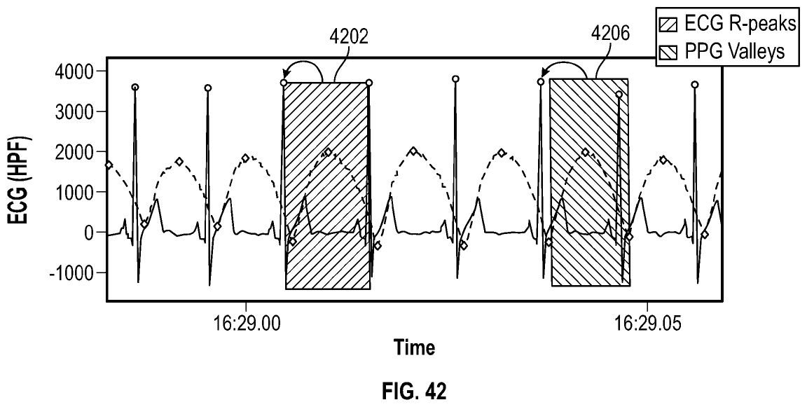

FIG. 42 illustrates a graph for calculating PAT as an integral using different integration intervals according to some examples. In some embodiments, the system can take integrals over each ECG beat 4202 (the time period between ECG R-peaks) or over each PPG beat 4206 (using valleys as starting/stopping times). Advantageously, the PAT determination is more robust to noisy PPG signals since the determination isn't reliant on a precise determinations of PPG peaks. One advantage of using the ECG beat is that calculating the integral over each ECG beat provides additional robustness over noisy PPG data with valleys that are different or difficult to precisely detect.

Each of the processes, methods, and algorithms described in the preceding sections may be embodied in, and fully or partially automated by, code modules executed by one or more computer systems or computer processors comprising computer hardware. The code modules may be stored on any type of non-transitory computer-readable medium or computer storage device, such as hard drives, solid state memory, optical disc, and/or the like. The systems and modules may also be transmitted as generated data signals (for example, as part of a carrier wave or other analog or digital propagated signal) on a variety of computer-readable transmission mediums, including wireless-based and wired/cable-based mediums, and may take a variety of forms (for example, as part of a single or multiplexed analog signal, or as multiple discrete digital packets or frames). The processes and algorithms may be implemented partially or wholly in application-specific circuitry. The results of the disclosed processes and process steps may be stored, persistently or otherwise, in any type of non-transitory computer storage such as, for example, volatile or non-volatile storage.

The various features and processes described above may be used independently of one another, or may be combined in various ways. All possible combinations and subcombinations are intended to fall within the scope of this disclosure. In addition, certain method or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed example examples. The example systems and components described herein may be configured differently than described. For example, elements may be added to, removed from, or rearranged compared to the disclosed example examples.

Conditional language, such as, among others, “can,” “could,” “might,” or “may,” unless specifically stated otherwise, or otherwise understood within the context as used, is generally intended to convey that certain examples include, while other examples do not include, certain features, elements and/or steps. Thus, such conditional language is not generally intended to imply that features, elements and/or steps are in any way required for one or more examples or that one or more examples necessarily include logic for deciding, with or without user input or prompting, whether these features, elements and/or steps are included or are to be performed in any particular example. The term “including” means “included but not limited to.” The term “or” means “and/or.”

Any process descriptions, elements, or blocks in the flow or block diagrams described herein and/or depicted in the attached figures should be understood as potentially representing modules, segments, or portions of code which include one or more executable instructions for implementing specific logical functions or steps in the process. Alternate implementations are included within the scope of the examples described herein in which elements or functions may be deleted, executed out of order from that shown or discussed, including substantially concurrently or in reverse order, depending on the functionality involved, as would be understood by those skilled in the art.

All of the methods and processes described above may be at least partially embodied in, and partially or fully automated via, software code modules executed by one or more computers. For example, the methods described herein may be performed by the computing system and/or any other suitable computing device. The methods may be executed on the computing devices in response to execution of software instructions or other executable code read from a tangible computer readable medium. A tangible computer readable medium is a data storage device that can store data that is readable by a computer system. Examples of computer readable mediums include read-only memory, random-access memory, other volatile or non-volatile memory devices, CD-ROMs, magnetic tape, flash drives, and optical data storage devices.

It should be emphasized that many variations and modifications may be made to the above-described examples, the elements of which are to be understood as being among other acceptable examples. All such modifications and variations are intended to be included herein within the scope of this disclosure. The foregoing description details certain examples. It will be appreciated, however, that no matter how detailed the foregoing appears in text, the systems and methods can be practiced in many ways. For example, a feature of one example may be used with a feature in a different example. As is also stated above, it should be noted that the use of particular terminology when describing certain features or aspects of the systems and methods should not be taken to imply that the terminology is being re-defined herein to be restricted to including any specific characteristics of the features or aspects of the systems and methods with which that terminology is associated.

Various examples of a physiological monitoring device, methods, and systems are disclosed herein. These various examples may be used alone or in combination, and various changes to individual features of the examples may be altered, without departing from the scope of the invention. For example, the order of various method steps may in some instances be changed, and/or one or more optional features may be added to or eliminated from a described device. Therefore, the description of the examples provided above should not be interpreted as unduly limiting the scope of the invention as it is set forth in the claims.

Various modifications to the implementations described in this disclosure may be made, and the generic principles defined herein may be applied to other implementations without departing from the spirit or scope of this disclosure. Thus, the scope of the disclosure is not intended to be limited to the implementations shown herein, but are to be accorded the widest scope consistent with this disclosure, the principles and the novel features disclosed herein.

Certain features that are described in this specification in the context of separate examples also can be implemented in combination in a single example. Conversely, various features that are described in the context of a single example also can be implemented in multiple examples separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, such operations need not be performed in the particular order shown or in sequential order, or that all illustrated operations be performed, to achieve desirable results. Further, the drawings may schematically depict one more example processes in the form of a flow diagram. However, other operations that are not depicted can be incorporated in the example processes that are schematically illustrated. For example, one or more additional operations can be performed before, after, simultaneously, or between any of the illustrated operations. Moreover, the separation of various system components in the examples described above should not be interpreted as requiring such separation in all examples. Additionally, other examples are within the scope of the following claims. In some cases, the actions recited in the claims can be performed in a different order and still achieve desirable results.

1. An electronic device configured to monitor physiological signals of a user, the electronic device comprising:
a housing at least partially enclosing a circuit board configured to process physiological signals to infer a physiological characteristic of the user;
a flexible wing extending from the housing and configured to be affixed to a surface of the user; and
an optical sensor assembly positioned on the flexible wing and configured to obtain a photoplethysmography signal, wherein the optical sensor assembly comprises an optical emitter configured to emit light and an optical detector configured to receive light, wherein at least a portion of the light emitted by the optical emitter is directed into the skin of the user and received by the optical detector from the skin of the user, wherein the optical sensor assembly further comprises a first directional layer between the optical emitter and the surface of the user and a second directional layer between the optical detector and the surface of the user, and wherein the first directional layer directs light from the optical emitter towards the surface of the user and the second directional layer directs light from the surface of the user towards the optical detector.

2. The electronic device of claim 1, wherein the first directional layer comprises a first convex lens and the second directional layer comprises a second convex lens.

3. The electronic device of claim 2, wherein the first convex lens and the second convex lens are positioned to extend below the flexible wing such that the first convex lens and the second convex lens press into skin of the user when the electronic device is affixed to the skin of the user.

4. The electronic device of claim 1, wherein the optical sensor assembly further comprises an opaque barrier positioned between the first directional layer and the second directional layer.

5. The electronic device of claim 4, wherein the opaque barrier blocks at least a portion of light emitted by the optical emitter from being received by the optical detector without passing through the skin of the user.

6. The electronic device of claim 4, wherein the opaque barrier is configured to direct at least a portion of light from the surface of the user towards the optical detector.

7. The electronic device of claim 4, further comprising a reflective layer positioned between the opaque barrier and the skin of the user.

8. The electronic device of claim 7, wherein the reflective layer reflects light from the first directional layer.

9. The electronic device of claim 4, wherein the first directional layer and the second directional layer protrudes beyond a portion of the opaque barrier overlapping at least the portion of the opaque barrier and increasing an area of contact with the skin of the user.

10. The electronic device of claim 1, wherein the first directional layer and the second directional layer are configured to direct or redirect passage of light of a particular wavelength.

11. The electronic device of claim 1, wherein the first directional layer comprises a first concave lens and the second directional layer comprises a second concave lens.

12. The electronic device of claim 11, wherein the first concave lens and the second concave lens are formed from a high index material that is index-matched to the stratum corneum of the chest of the user.

13. The electronic device of claim 1, wherein the first directional layer comprises a first half ball lens and the second directional layer comprises a second half ball lens.

14. The electronic device of claim 13, wherein the first half ball lens and the second half ball lens each comprise a sapphire half ball lens.

15. The electronic device of claim 13, further comprising a first adhesive layer configured to affix the first half ball lens to the optical emitter and a second adhesive layer configured to affix the second half ball lens to the optical detector.

16. The electronic device of claim 1, wherein the circuit board comprises a flex circuit board.

17. The electronic device of claim 1, further comprising a backing substrate that exerts pressure on the circuit board to increase contact between the optical emitter and the skin of the user, to increase contact between the optical detector and the skin of the user, or to increase contact between the optical emitter and the skin of the user and the optical detector and the skin of the user.

18. The electronic device of claim 1, wherein the optical sensor assembly comprises a plurality of optical emitters including the optical emitter, and wherein when the flexible wing is affixed to the surface of the user, the plurality of optical emitters pinch skin of the user causing a portion of skin to be positioned between the plurality of optical emitters such that the plurality of optical emitters focus light toward the portion of skin positioned between the plurality of optical emitters.

19. The electronic device of claim 1, wherein the optical sensor assembly comprises a plurality of optical detectors including the optical detector, and wherein when the flexible wing is affixed to the surface of the user, the plurality of optical detectors pinch skin of the user causing a portion of skin to be positioned between the plurality of optical detectors such that the plurality of optical detectors receive light from the portion of skin positioned between the plurality of optical detectors.

20. The electronic device of claim 1, wherein when the flexible wing is affixed to the surface of the user, the optical emitter and the optical detector pinch the skin of the user causing a portion of skin to be positioned between the optical emitter and the optical detector to direct light from the optical emitter through the skin of the user to the optical detector.

21. The electronic device of claim 1, wherein the optical sensor assembly further comprises a fiber optical cable or light guide configured to direct light from the optical emitter towards the skin of the user.

22. The electronic device of claim 1, further comprising a spring configured to exert pressure to increase contact between the optical emitter and skin of the user and to increase contact between the optical detector and the skin of the user.

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)
